(19) United States
(12) Patent Application Publication
(10) Pub. No.: US 2021/0171961 A1
(43) Pub. Date: Jun. 10, 2021

(54) SELECTION AND GENETIC MODIFICATION OF PLANT ASSOCIATED METHYLOBACTERIUM

(71) Applicant: NEWLEAF SYMBIOTICS, INC., St. Louis, MO (US)

(72) Inventors: Janne KEROVUO, Los Angeles, CA (US); Natalie BREAKFIELD, St. Louis, MO (US); Quan ZHANG, St. Louis, MO (US); Desmond JIMENEZ, St. Louis, MO (US); Gregg BOGOSIAN, St. Louis, MO (US)

(21) Appl. No.: 17/255,270

(22) PCT Filed: Jul. 3, 2019
(86) PCT No.: PCT/US2019/040620

§ 371 Date: Dec. 22, 2020

Related U.S. Application Data
(60) Provisional application No. 62/694,775, filed on Jul. 6, 2018.
(60) Provisional application No. 62/760,092, filed on Nov. 13, 2018.
(60) Provisional application No. 62/802,805, filed on Feb. 8, 2019.
(60) Provisional application No. 62/819,023, filed on Mar. 15, 2019.

(51) Int. Cl. C12N 15/74; A01N 63/20; C12N 1/20

(52) U.S. Cl. CPC C12N 15/74; C12N 1/20; A01N 63/20

(57) ABSTRACT

Methods for generating transformed Methlyobacterium isolates are provided. Such methods can be used to develop novel Methylobacterium isolates having improved properties for use in a variety of industrial applications.

### CROSS-REFERENCE TO RELATED APPLICATIONS

This patent application claims the benefit of U.S. 62/694,775, filed Jul. 6, 2018, U.S. 62/760,092, filed Nov. 13, 2018, U.S. 62/819,023, filed Mar. 15, 2019, and U.S. 62/802,805, filed Feb. 8, 2019, which are each incorporated herein by reference in their entireties.

### SEQUENCE LISTING STATEMENT

A sequence listing containing the file named “53907_188935_ST25.txt” which is 63566 bytes (measured in MS-Windows®) and created on Jun. 25, 2019, contains 62 nucleotide sequences and 11 amino acid sequences, is provided herewith via the USPTO's EFS system, and is incorporated herein by reference in its entirety.

### BACKGROUND

Bacterial species of the genus Methylobacterium are facultative methylotrophs that can use one-carbon organic compounds such as methane or methanol as a carbon source for growth, but also have the ability to utilize larger organic compounds, such as organic acids, higher alcohols, sugars, and the like. Some methylotrophic bacteria of the genus Methylobacterium are pink-pigmented and often referred to as PPFM bacteria, for pink-pigmented facultative methylotrophs, although not all species of the genus are pink. For example, M. nodulans is a nitrogen-fixing Methylobacterium and the type strain for this species in not pink (Sy et al., 2001). Methylobacterium have been found in soil, dust, fresh water, sediments, and leaf surfaces, as well as in industrial and clinical environments (Green, 2006). Over 50 species of Methylobacterium have been described, however, at least one recent study suggests that some species be reclassified into a new genus, Methylorubrum (Green and Ardley (2018)).

Methylobacterium bacteria are symbiotic epiphytic bacteria that can in some instances successfully colonize different plant tissues. They are of interest for a number of commercial applications including as agricultural treatments for plant health promotion and biopesticidal activity (US Patent Application Publications US20160302423, US20160295868, and US20180295841; WO/2018/106899), for use in bioreactors for a production of chemical bioproducts including PHA, PHB and other value-added chemicals (Bourque et al. (1995), Cui et al. (2018), U.S. Pat. No. 8,956,835), for environmental applications such as bioremediation (Yonemitsu et al. (2016); Salam et al. (2015)), and in aquaculture where value as fish feed is based on the ability of Methylobacterium bacteria to produce carotenoids (Hardy et al. (2018)). There is a need in the industry for novel Methylobacterium isolates that have traits that are advantageous in agriculture, in bioreactors for production of chemicals or Methylobacterium biomass, and in bioremediation.

Small non-coding double-stranded RNAs play a central role in RNA silencing pathways in plants. This endogenous pathway for downregulation of gene expression is known as RNA interference (RNAi). RNAi represents diverse RNA-based processes that all result in sequence-specific inhibition of gene expression at the transcriptional, post-transcriptional or translational level. It has emerged as powerful highly effective mechanism for gene silencing in plants or insects. RNAi has been successfully used in transgenic plants to decrease expression of endogenous genes and to engineer resistance to viral, fungal and bacterial pathogens as well as to provide protection from nematode and insect pests. For RNAi-based crop improvement strategies, a significant challenge is the delivery of active dsRNA molecules to trigger the activation of the RNAi pathway. Gram-negative Escherichia coli bacteria can be engineered to produce interfering dsRNA, which, when ingested by the nematode Caenorhabditis elegans, can induce systemic gene silencing.

### SUMMARY

Methods of producing a transconjugant Methylobacterium isolate, comprising:

incubating (i) a donor Methylobacterium isolate comprising a mobilizable plasmid containing a marker; and (ii) a recipient Methylobacterium isolate; wherein the mobilizable plasmid has an origin of replication functional in the recipient Methylobacterium isolate; wherein said mobilizable plasmid is transferred from said donor Methylobacterium isolate to said recipient Methylobacterium isolate; and screening cells of said recipient Methylobacterium isolate for the presence of the mobilizable plasmid marker to identify a transconjugant Methylobacterium isolate are provided.

Methods of producing a population of transconjugant Methylobacterium isolates, comprising the steps of (i) incubating a composition comprising a first donor Methylobacterium isolate comprising a mobilizable plasmid containing an origin of replication functional in Methylobacterium and a marker, and one or more recipient Methylobacterium isolates under conditions wherein said mobilizable plasmid is transferred from said donor Methylobacterium isolate to said recipient Methylobacterium isolate or isolates; and (ii) screening cells of said recipient Methylobacterium isolate or isolates for the presence of the mobilizable plasmid marker to identify one or more transconjugant Methylobacterium isolates are provided.

Methods of producing a transformed Methylobacterium isolate, comprising:

transforming a recipient Methylobacterium isolate with a plasmid having an origin of replication functional in the recipient Methylobacterium isolate and a marker; wherein said plasmid is transferred to said recipient Methylobacterium isolate; and screening cells of said recipient Methylobacterium isolate for the presence of the marker to identify a transformed Methylobacterium isolate are provided.

Methylobacterium comprising a recombinant DNA construct wherein a promoter is operably linked to a heterologous sequence encoding a nucleic acid that can trigger an RNAi response are provided. Compositions comprising the aforementioned Methylobacterium are also provided. In certain embodiments, the compositions can further comprise at least one agriculturally acceptable excipient and/or adjuvant.

Transformed Methylobacterium strains that comprise a selected host Methylobacterium strain or variant thereof comprising: (i) a first recombinant DNA construct wherein a promoter is operably linked to at least one heterologous sequence encoding a nucleic acid that can trigger an RNAi response, and (ii) a second recombinant DNA construct wherein a promoter is operably linked to a heterologous sequence comprising a nucleic acid that encodes a pesticidal or herbicide tolerance protein are provided. Compositions comprising the transformed Methylobacterium are also provided. In certain embodiments, the compositions can further comprise at least one agriculturally acceptable excipient and/or adjuvant.

Plant parts which are coated or at least partially coated with the aforementioned Methylobacterium, compositions comprising the Methylobacterium, transformed Methylobacterium, or compositions comprising the transformed Methylobacterium are provided. In certain embodiments, the plant parts are seeds, leaves, roots, stems, tubers, flowers, or fruits.

Methods of altering a phenotypic trait in a host plant comprising the step of applying the aforementioned Methylobacterium, compositions comprising the Methylobacterium, transformed Methylobacterium, or compositions comprising the transformed Methylobacterium to a plant or a plant part are provided.

Methods inhibiting a plant pest or plant pathogen in a host plant comprising the step of applying the aforementioned Methylobacterium, compositions comprising the Methylobacterium, transformed Methylobacterium, or compositions comprising the transformed Methylobacterium to a plant or a plant part are provided.

Methods of detecting the presence of (a)Methylobacterium strain NLS0042 or a variant thereof; or (b) NLS0064 a variant thereof in a sample comprising detecting the presence in the sample of a nucleic acid comprising or located within: (i) SEQ ID NO:14, 15, and/or 16; or (ii) SEQ ID NO: 17, 18, or 19, respectively, are provided.

### DRAWINGS

FIG. 1. Mobilizable plasmid pQZ1024.

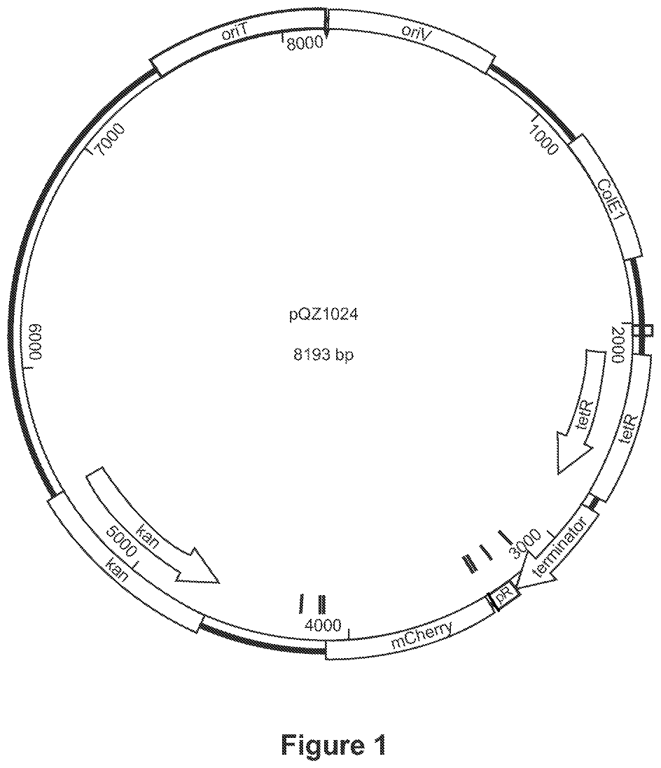

### DESCRIPTION

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where a term is provided in the singular, embodiments comprising the plural of that term are also provided.

As used herein, the terms “include,” “includes,” and “including” are to be construed as at least having the features or encompassing the items to which they refer while not excluding any additional unspecified features or unspecified items.

As used herein, a “host Methylobacterium strain” refers to a Methylobacterium strain which lacks recombinant DNA. In certain embodiments, the host Methylobacterium serves as a recipient for heterologous DNA or for recombinant DNA to provide an engineered Methylobacterium.

As used herein, the term “Methylobacterium” refers to genera and species in the methylobacteriaceae family, including bacterial strains in the Methylobacterium genus and the proposed Methylorubrum genus (Green and Ardley (2018)). Methylobacterium includes pink-pigmented facultative methylotrophic bacteria (PPFM) and also encompasses the non-pink-pigmented Methylobacterium nodulans, as well as colorless mutants of Methylobacterium isolates such as described herein. For example, and not by way of limitation, “Methylobacterium” refers to bacteria of the species listed below as well as any new species that have not yet been reported or described that can be characterized as Methylobacterium or Methylorubrum based on phylogenetic analysis.

“Methylobacterium” includes, but is not limited to: Methylobacterium adhaesivum; Methylobacterium oryzae; Methylobacterium aerolatum Methylobacterium oxalidis; Methylobacterium aquaticum; Methylobacterium persicinum; Methylobacterium brachiatum; Methylobacterium phyllosphaerae; Methylobacterium brachythecii; Methylobacterium phyllostachyos; Methylobacterium bullatum; Methylobacterium platani; Methylobacterium cerastii; Methylobacterium pseudosasicola; Methylobacterium currus Methylobacterium radiotolerans; Methylobacterium dankookense; Methylobacterium soli; Methylobacterium frigidaeris; Methylobacterium specialis; Methylobacterium fujisawaense; Methylobacterium tardum; Methylobacterium gnaphalii; Methylobacterium tarhaniae; Methylobacterium goesingense; Methylobacterium thuringiense; Methylobacterium gossipiicola; Methylobacterium trifolii; Methylobacterium gregans; Methylobacterium variabile; Methylobacterium haplocladii; Methylobacterium (Methylorubrum) aminovorans; Methylobacterium hispanicum; Methylobacterium (Methylorubrum) extorquens; Methylobacterium indicum; Methylobacterium (Methylorubrum) podarium; Methylobacterium iners; Methylobacterium(Methylorubrum) populi; Methylobacterium isbiliense; Methylobacterium (Methylorubrum) pseudosasae; Methylobacterium jeotgali: Methylobacterium (Methylorubrum) rhodesianum; Methylobacterium komagatae; Methylobacterium (Methylorubrum) rhodinum; Methylobacterium longum; Methylobacterium (Methylorubrum) salsuginis; Methylobacterium marchantiae; Methylobacterium (Methylorubrum) suomiense; Methylobacterium mesophilicum; Methylobacterium (Methylorubrum) thiocyanatum; Methylobacterium nodulans; Methylobacterium (Methylorubrum) zatmanii; and Methylobacterium organophilum.

As used herein, the term “strain” shall include all isolates of such strain.

As used herein, the phrase “mobilizable plasmid” refers to a plasmid that can be transferred from a donor strain to a recipient strain. A mobilizable plasmid as defined herein contains cis-acting DNA elements (i.e. oriT) required for conjugation and has an origin of replication functional in Methylobacterium. Other elements required for conjugation may also be encoded on a mobilizable plasmid. A conjugative or self-transmissible plasmid contains cis-acting DNA required for conjugation and encodes all of the genes required for DNA transfer to a recipient cell/strain or isolate, and is also considered a mobilizable plasmid for use in methods defined herein.

As used herein, the phrase “helper plasmid” refers to a plasmid that encodes trans-acting proteins required for transformation. Generally, a helper plasmid used herein is maintained in E. coli, a species that either does not form colonies on AMS-MC (ammonia mineral salt—methanol cycloheximide) plates used for growth of Methylobacterium strains, or can be distinguished from Methylobacterium strains as being transparent in color as opposed to the pink colonies formed by most Methylobacterium species.

As used herein, the phrase “donor Methylobacterium isolate” refers to an isolate that contains a mobilizable plasmid that can be introduced into a recipient Methylobacterium isolate.

As used herein, the phrase “recipient Methylobacterium isolate” refers to an isolate which can receive a mobilizable plasmid via conjugative transfer from a donor Methylobacterium isolate. A recipient Methylobacterium isolate is also used herein to refer to a Methylobacterium isolate that has been transformed by electroporation or other means to contain non-native plasmid DNA.

As used herein, the phrase “transconjugant Methylobacterium isolate” refers to an isolate which has been generated by transfer of a mobilizable plasmid from a donor Methylobacterium isolate to a recipient Methylobacterium isolate. A transconjugant Methylobacterium isolate may be identified by the presence of a marker on the mobilizable plasmid.

As used herein, a “transformed Methylobacterium” refers to a Methylobacterium strain which has been modified to contain heterologous DNA. Transformed Methylobacterium include isolates that have been modified to contain a foreign DNA plasmid, either by direct DNA uptake of the foreign plasmid, for example by electroporation, heat shock, ultra-sound, transduction (e.g. bacteriophage) or by conjugation from a donor bacterium, and transconjugant Methylobacterium isolates. Heterologous DNA can, in some embodiments, be recombinant DNA. Such heterologous DNA can be present on a plasmid or integrated into the chromosome of the transformed Methylobacterium. Transformed Methylobacterium include engineered Methylobacterium.

As used herein, the term “isolate” refers to the subject Methylobacterium as well as to the progeny or potential progeny of the subject Methylobacterium.

As used herein, “variant” when used in the context of a Methylobacterium isolate, refers to any isolate that has chromosomal genomic DNA with at least 99%, 99.9, 99.8, 99.7, 99.6%, or 99.5% sequence identity to chromosomal genomic DNA of a deposited Methylobacterium isolate provided herein, and has a plant beneficial trait of the deposited isolate. A variant of an isolate can be obtained from various sources including soil, plants or plant material and water, particularly water associated with plants and/or agriculture. Variants include derivatives obtained from deposited isolates. Variants also include strains obtained from a host Methylobacterium strain by genetic transformation, mutagenesis and/or insertion of a heterologous sequence.

As used herein, “derivative” when used in the context of a Methylobacterium isolate, refers to any strain that is obtained from a deposited Methylobacterium isolate provided herein. Derivatives of a Methylobacterium isolate include, but are not limited to, derivatives of the strain obtained by selection, derivatives of the strain selected by mutagenesis and selection, and genetically transformed strains obtained from the Methylobacterium isolate. A “derivative” can be identified, for example based on genetic identity to the strain from which it was obtained and will generally exhibit chromosomal genomic DNA with at least 99%, 99.9, 99.8, 99.7, 99.6%, or 99.5% sequence identity to chromosomal genomic DNA of the strain from which it was derived.

As used herein, the term “triparental mating” refers to a process for transfer of a plasmid from a donor isolate to a recipient isolate in which a helper plasmid is required for conjugation to occur. In this type of conjugation, donor cells, recipient cells, and a “helper strain” participate. The donor isolate comprises a mobilizable plasmid and the helper strain contains a plasmid that encodes trans-acting proteins required for conjugation.

As used herein, the term “marker” refers to a genetic sequence and/or an encoded product of the genetic sequence, that can be used to identify transformed Methylobacterium strains that contain the marker. In some instances, a marker can be a selectable marker, such as a gene encoding a protein conferring resistance to an antibiotic, or a screenable marker, such as a gene that encodes a fluorescent protein or other reporter protein that can be visualized to identify a recipient Methylobacterium isolate. A marker may also be a genetic element that is present in a donor Methylobacterium isolate or in a DNA solution comprising a plasmid, but not in a recipient Methylobacterium isolate, that can be identified in transconjugant isolates by genetic analysis, for example by use of DNA primers.

As used herein, the phrase “native Methylobacterium plasmid” refers to a plasmid naturally present in a Methylobacterium strain obtained from an environmental source.

As used herein, the phrase “recombinant plasmid” refers to a plasmid that has been manipulated outside of a Methylobacterium isolate to produce a plasmid that can be introduced into a donor Methylobacterium isolate and transferred by conjugation as described herein to a recipient Methylobacterium isolate, or introduced directly into a recipient Methylobacterium isolate by electroporation.

As used herein the term “foreign plasmid” refers to a plasmid that is transferred to a recipient Methylobacterium isolate that was not present in the recipient Methylobacterium isolate prior to transformation. A foreign plasmid as used herein may be one or more of specific types of plasmids defined herein, including a mobilizable plasmid, a native Methylobacterium plasmid or a recombinant plasmid. A foreign plasmid may also be a natural plasmid from a bacterial species other than Methylobacterium.

As used herein, the term “colonize” refers to the ability of Methylobacterium to grow and reproduce in an environment, such as a plant, plant part or soil. For example, a Methylobacterium is considered to colonize a plant or plant part if it can survive and grow on or inside the plant or plant part, including inside a plant cell.

As used herein the term “colonization efficiency” refers to the relative ability of a given Methylobacterium strain to colonize a plant host cell or tissue as compared to non-colonizing control samples or other Methylobacterium strains. Colonization efficiency can be assessed, for example and without limitation, by determining colonization density, reported for example as colony forming units (CFU) per mg of plant tissue, or by quantification of nucleic acids specific for a strain in a colonization screen, for example using qPCR.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Transformation methods to generate novel Methylobacterium strains are described herein. In some embodiments, a Methylobacterium strain or isolate for use in such methods is selected prior to transformation based on performance in a number of screens that evaluate the fitness of a Methylobacterium for use as an agricultural inoculant Such screens include screens for tolerance to desiccation, tolerance to agricultural chemicals, colonization efficiency on a target plant and/or plant part, and screens for growth rate and ease of production when grown in media with varying sources of carbon, nitrogen and other nutrients, such as vitamins or other trace elements. In some embodiments, such screens can also be used to identify transformed strains having improved performance in such screens.

In some embodiments a screen for desiccation tolerance is employed. Methods for identifying desiccation tolerant Methylobacterium include screening a population of Methylobacterium for viability after a period of drying, for example, in one embodiment drying under a laminar flow hood, and comparing viability to other tested strains. In some embodiments, Methylobacterium can be dried directly from the growth medium, for example, in one embodiment, dried in petri dishes or microtiter plates. In some embodiments, the Methylobacterium are grown in media having a single carbon source, dried in the minimal media and rehydrated in a rich nutrient media. In some embodiments, Methylobacterium are coated on seeds and allowed to dry and tested for viability after a period of storage on dry seeds. In some embodiments, microorganisms are stored on dry seeds from one day to three weeks, including 2 days, 5 days, 1 week, 2 weeks, and 3 weeks or more before testing for viability. In some embodiments, microorganisms are stored on dry seeds for greater than 4 weeks prior to testing for viability. In some embodiments, Methylobacterium are tested for production of exopolysaccharide (EPS) which has been shown to be involved in protection from desiccation (Gasser et al. (2009). In certain embodiments, Methylobacterium isolates or strains are selected for improved desiccation tolerance in comparison to a control Methylobacterium. In certain embodiments, a selected host Methylobacterium strain or variant thereof exhibits or is selected for improved desiccation tolerance in comparison to a control Methylobacterium. In certain embodiments the control Methylobacterium is a parental Methylobacterium isolate or strain which has not been subjected to mutagenesis, conjugation, or transformation. In certain embodiments, the control Methylobacterium is a Methylobacterium isolate or strain which has a desiccation tolerance (DT) score of 1.4 or less. The desiccation tolerance (DT) score is obtained by subjecting the test Methylobacterium and control Methylobacterium to drying conditions (e.g., placement in a sterile laminar flow hood environment for about 6, 7, 8, or more hours), determining the percent viability of the Methylobacterium after drying by comparing titers of equal aliquots of undried and dried Methylobacterium, and multiplying the percent viability by 0.03 to obtain a DT score. Such DT scores can typically fall between 0 and 3. Methylobacterium with a DT value of greater than or equal to 1.5 can be considered desiccation tolerant.

In some embodiments, a screen for the ability of a Methylobacterium to tolerate the presence of commonly used agricultural chemicals is used. In some embodiments, Methylobacterium to be tested for tolerance to agricultural chemicals will be grown in liquid media and spotted onto solid media plates containing the agricultural chemicals. In some embodiments, the agricultural chemicals in such a screen will include herbicides, for example one or more of the following acetochlor, clethodim, dicamba, flumioxazin, fomesafen, glyphosate, glufosinate, mesotrione, quizalofop, saflufenacil, sulcotrione, and 2,4-D. In some embodiments, the agricultural chemicals in such a screen will include fungicides, for example one or more of the following acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole. In some embodiments, the agricultural chemicals in such a screen will include insecticides and/or nematicides, including, for example abamectin, aldicarb, aldoxycarb, bifenthrin, carbofuran, chlorantraniliporle, chlothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodichlofen, spirotetramat, tefluthrin, thiacloprid, thiamethoxam, tioxazafen, and thiodicarb. In some embodiments, the agricultural chemicals in such a screen will include biocides, such as isothiazolinones, including for example 1,2 Benzothiazolin-3-one (BIT), 5-Chloro-2-methyl-4-isothiazolin-3-one (CIT), 2-Methyl-4-isothiazolin-3-one (MIT), octylisothiazolinone (OIT), dichlorooctylisothiazolinone (DCOIT), and butylbenzisothiazolinone (BBIT); 2-Bromo-2-nitro-propane-1,3-diol (Bronopol), 5-bromo-5-nitro-1,3-dioxane (Bronidox), Tris(hydroxymethyl)nitromethane, 2,2-Dibromo-3-nitrilopropionamide (DBNPA), and alkyl dimethyl benzyl ammonium chlorides. In some embodiments, the agricultural chemicals in such a screen will include any combination of fungicides, herbicides, insecticides nematicides, and biocides. In certain embodiments, Methylobacterium isolates or strains are selected for improved agricultural chemistry tolerance in comparison to a control Methylobacterium. In certain embodiments, a selected host Methylobacterium strain or variant thereof exhibits or is selected for improved agricultural chemistry tolerance in comparison to a control Methylobacterium. In certain embodiments the control Methylobacterium is a parental Methylobacterium isolate or strain which has not been subjected to mutagenesis, conjugation, or transformation. In certain embodiments, agricultural chemistry tolerance can be assigned a rating of 0-3, where 0 represents no growth (no tolerance) and 3 representing full growth (i.e., growth equivalent to growth on control media lacking the agricultural chemical(s)). In certain embodiments, strains with a score of greater than or equal to 1.66 are agricultural chemical tolerant and strains with a score of less than 1.66 are agricultural chemistry intolerant.

In some embodiments, a screen for the ability of a Methylobacterium to grow robustly and to a high titer in media comprising varying sources, concentrations and combinations of carbon, nitrogen and other nutrients, including one or more vitamins or other trace elements, is employed. Such screens find particular interest, for example, where a desirable plant-associated microorganism is known to have a relatively slow growth rate.

In some embodiments, a colonization screen is employed, and Methylobacterium is applied in the screen at a lower dose than typically used in agriculture in order to identify strains that would provide an advantage for commercial production. In some embodiments, a dose of 10² to 10⁸ CFU is applied to a plant or plant part. In some embodiments, a dose of 10³, 10⁴, 10⁵, 10⁶, or 10⁷ CFU is applied to a plant or plant part. In some embodiments, the dose is applied to a plant root, leaf, stem, seed, fruit or flower. In some embodiments, a single Methylobacterium strain will be assayed and compared to a control strain. In some embodiments, two or more strains will be screened in a colonization assay with or without a control treatment to determine relative colonization efficiency of the strains in the screen. In some embodiments, a population of Methylobacterium in a colonization screen will comprise two or more strains to be tested and a control, where the control can be a treatment where no Methylobacterium is added to a sample, a treatment where a Methylobacterium known or previously determined to be a poor colonizer of the target plant host is used, or a treatment where a Methylobacterium known or previously determined to be an efficient colonizer of the target plant host is used. In some embodiments, both a control lacking added Methylobacterium strain and a control Methylobacterium strain known to be a poor colonizer may be used. In certain embodiments, Methylobacterium isolates or strains are selected for improved colonization efficiency in comparison to a control Methylobacterium. In certain embodiments, a selected host Methylobacterium strain or variant thereof exhibits or is selected for improved colonization efficiency in comparison to a control Methylobacterium. In certain embodiments the control Methylobacterium is a parental Methylobacterium isolate or strain which has not been subjected to mutagenesis, conjugation, or transformation. In certain embodiments the control Methylobacterium is a Methylobacterium isolate or strain which is known or shown herein to exhibit poor, average, or above average colonization efficiency. In certain embodiments, the selected Methylobacterium, selected host Methylobacterium strain, or variant thereof provides for an increase in Colony Forming Units (CFUs) per milligram (mg) of plant or plant part fresh weight in comparison to a control. In certain embodiments, the selected Methylobacterium, selected host Methylobacterium strain, or variant thereof exhibits at least a 7-fold, 8-fold, 10-fold, or 13-fold increase in CFUs per mg of plant fresh weight than the control strain.

In one embodiment, a novel transformed Methylobacterium isolate is prepared by electroporation of a selected Methylobacterium strain with DNA comprising a plasmid capable of replication in Methylobacterium wherein said plasmid encodes one or more useful traits for improvement of said Methylobacterium isolate. In other embodiments, a novel transconjugant Methylobacterium isolate is prepared by conjugation between a donor Methylobacterium isolate and a recipient Methylobacterium isolate, whereby one or more plasmids from said donor Methylobacterium isolate is transferred to the recipient Methylobacterium isolate to generate a transconjugant Methylobacterium isolate.

Bacterial conjugation involves direct introduction of one or more plasmids from a donor cell to a recipient cell and involves mixing or incubating donor and recipient cells so that they are in direct contact. Components required for conjugation include a cis-acting DNA element comprising an origin of transfer (oriT), and trans-acting proteins including a relaxase that cleaves plasmid DNA at oriT, a Type IV secretion system (T4SS) involved in mating pair formation, and coupling proteins. Conjugation occurs by the formation of a cytoplasmic connection via extracellular pili between the donor and the recipient. A cell of a donor isolate produces a pilus which attaches to a cell of a recipient isolate. Plasmid DNA is nicked at a specific site in oriT by relaxase which binds to the DNA strand and facilitates transfer of the DNA to the recipient cell either alone, or as part of a multi-protein relaxosome complex. The plasmid DNA strand is replicated in the recipient cell to complete the conjugation process.

In some cases, a plasmid to be transferred contains the cis-acting oriT and encodes all proteins required for transfer to a recipient cell. Such plasmids are referred to as self-transmissible. In other cases, a plasmid may contain oriT but does not encode one or more trans-acting proteins required for conjugative transfer. Such a plasmid is referred to as mobilizable and can be transferred to a recipient cell if all trans-acting components of conjugation are provided by another source. The trans-acting components may be encoded on the chromosome of the donor isolate, or the source of trans-acting components may be a helper plasmid that promotes the transfer of a mobilizable plasmid by providing any required trans-acting conjugation components that are not encoded on the mobilizable plasmid. A helper plasmid may be present in the donor isolate or may be provided in a helper bacterial strain.

In one embodiment, methods provided herein find use in production of Methylobacterium strains having genetic variability as the result of conjugative exchange of plasmids between two or more different Methylobacterium isolates. Methods as described herein, in some embodiments, result in introduction of non-native DNA to recipient Methylobacterium isolates. In some embodiments a transformed Methylobacterium isolate can have traits that are advantageous in agriculture, in bioreactors for production of chemicals or Methylobacterium biomass, and in bioremediation. In one embodiment, conjugated plasmids that result in useful traits are identified and may be isolated from a transformed Methylobacterium and used in additional transformation methods, such as electroporation, to generate additional transformed Methylobacterium strains.

In some embodiments, a donor Methylobacterium isolate used in conjugation methods described herein will contain a recombinant mobilizable plasmid that is introduced into the donor Methylobacterium isolate by conjugation using an E. coli donor strain, or by electroporation, heatshock, ultrasound, or other similar methods commonly used to transform bacteria. In certain embodiments, transformation of Methylobacterium by electroporation can also be achieved as by previously described methods, including those set forth in Ueda et al. (1991), or adaptations thereof. In other embodiments, a mobilizable plasmid in a donor Methylobacterium isolate is a native Methylobacterium plasmid. An origin of replication functional in Methylobacterium on the mobilizable plasmid may be from a broad host range plasmid, for example oriV from an RK2 based plasmid, or may be an origin of replication native to a donor Methylobacterium isolate. In some embodiments, the origin of replication will also be functional in other bacteria, such as E. coli. In some embodiments, a donor Methylobacterium isolate is selected based on the identification of genes required for conjugative transfer on the chromosome or plasmids in the genome of the Methylobacterium isolate. Of particular interest are Methylobacterium isolates having all proteins required for conjugative transfer encoded on plasmids, including relaxase and T4SS proteins encoded on plasmids present in an isolate.

The mobilizable plasmid of the donor Methylobacterium isolate will have a marker that can be used to identify transconjugants containing the marker. As described in more detail below, in one embodiment, a marker can be a genetic sequence marker which can be used to identify transconjugants. In other embodiments, a marker may be a selectable marker or a screenable marker.

In some embodiments, a recipient Methylobacterium isolate used in methods described herein is taxonomically classified as in a different species from the donor Methylobacterium isolate. In other embodiments, the recipient and donor Methylobacterium isolates are classified as in the same taxonomic species. In some embodiments, the recipient Methylobacterium isolate will also contain plasmids with an origin of replication functional in Methylobacterium and encoding functions required for conjugative transfer. In such embodiments, conjugative transfer can occur from donor to recipient and/or from recipient to donor and transconjugants can be identified from either or both Methylobacterium isolates used in the conjugation methods.

In some embodiments, recipient Methylobacterium isolates can be visually distinguished from donor Methylobacterium isolates to aid in identification of recipient transconjugants. In one embodiment, a recipient Methylobacterium isolate will have a different morphology from the donor Methylobacterium isolate, such as forming larger colonies, having colonies of a darker or lighter shade of pink, having a colony surface that is more rough or smooth, and the like. In one embodiment, a recipient Methylobacterium isolate has a mutation in a carotenoid pathway gene such that white colonies are formed rather than the pink colonies of most Methylobacterium strains. In one embodiment, a recipient Methylobacterium isolate having a white colony phenotype is provided having a deletion mutation in the crtI gene.

In some embodiments in the methods described herein, a mobilizable plasmid in a donor Methylobacterium isolate or a foreign plasmid for use in electroporation contains a marker that can be used for detection of transformants of the recipient Methylobacterium isolate. In some embodiments, a marker is a genetic sequence on a donor Methylobacterium isolate that is not present in the recipient Methylobacterium isolate prior to conjugation. In one embodiment, a mobilizable plasmid in a donor Methylobacterium isolate or a foreign plasmid for use in electroporation is a native Methylobacterium plasmid. In one such embodiment, recipient Methylobacterium isolates in a conjugation mixture that contain the foreign native Methylobacterium plasmid are identified, for example, by colony morphology and screened for the presence of the genetic sequence marker to identify transconjugant isolates. In other embodiments, a mixture of recipient Methylobacterium isolates resulting from direct transformation, for example by electroporation as described herein, with a foreign native Methylobacterium plasmid is screened for the presence of the marker to identify transformed isolates. In one embodiment, screening is conducted by qPCR on individual recipient Methylobacterium isolate colonies from a conjugation mixture or resulting from electroporation with a foreign native Methylobacterium plasmid. The genetic sequence marker can vary in size, depending on the detection method, and may be from 50-1000 nucleotides in length. In one embodiment, nucleotide primers are used for amplification of a marker sequence to facilitate detection. In one embodiment, nucleotide primers are from 15-25 nucleotides in length.

Further embodiments described herein involve methods where a marker is a selectable or screenable marker. Such markers find particular use in methods where the mobilizable plasmid or foreign plasmid for electroporation is not a native Methylobacterium plasmid. Selectable markers can be genes which encode proteins that confer resistance to antibiotics, thus allowing detection of isolates containing the marker by the ability to grow in media containing antibiotics. Selectable markers useful in the methods described herein are well known in the art and include those conferring resistance to kanamycin, gentamycin, ampicillin, and chloramphenicol as well as other antibiotics known to be active against gram negative bacteria. Screenable markers, also referred to as reporter genes, encode proteins that cause a change in visible characteristics of a bacterial colony, particularly a change in color. Examples of screenable markers that find use in the methods described herein are lacZ, GUS, GFP, mcherry, and the like.

In some embodiments, expression of a selectable or screenable marker is provided by a recombinant construct on a mobilizable plasmid in the donor Methylobacterium isolate or on a foreign plasmid for use in electroporation. A promoter for driving expression of selectable or screenable marker gene can be any promoter functional in Methylobacterium and can include constitutive or inducible promoters. Exemplary promoters include promoters from phage such as the phage P_{R}, T5 and Sp6 promoters, promoters from lac and trp operons and native Methylobacterium promoters, including the promoter for methanol dehydrogenase mxaFI and others, such as described by Zhang and Lidstrom (2003).

In methods described herein, in one embodiment, transconjugant Methylobacterium isolates are obtained by incubating a donor Methylobacterium isolate comprising a mobilizable plasmid and an origin or replication functional in Methylobacterium, and a recipient Methylobacterium isolate resulting in transfer of a mobilizable plasmid from the donor Methylobacterium isolate to the recipient Methylobacterium isolate. Cells from the recipient Methylobacterium isolate resulting from said conjugation are screened to identify the transconjugant cells that contain one or more plasmids from the donor Methylobacterium isolate.

Cultures of donor and recipient Methylobacterium isolates are prepared and combined, typically at a ratio of 1:1, although the ratio may be varied to optimize conjugation between certain strains. In some embodiments, a higher ratio of donor to recipient Methylobacterium isolate will results in a higher number of transconjugants. In other embodiments, a higher ratio of recipient to donor Methylobacterium isolates will result in a higher number of conjugants. Thus, the donor:recipient or recipient:donor ratio for optimal conjugation may be 1:1, 2:1, 3:1, 4:1, 5:1, 10:1, 20:1, 50:1 or even 100:1. The donor:recipient Methylobacterium isolate mixture is grown on solid or in liquid media to allow conjugation to occur. In one embodiment, a mixture is plated on solid media and allowed to grow overnight. The conjugation mixture is harvested after growth, for example by scraping from a solid surface or centrifuging from a liquid media and plated to allow formation of individual colonies from which transconjugants can be identified. In one embodiment, AMS-MC (ammonia mineral salt—methanol cycloheximide) plates are used to grow the conjugation mixture.

In some embodiments, a conjugation mixture includes a helper E. coli strain that carries a conjugative plasmid encoding genes required for conjugation and DNA transfer. Where a helper strain is used, the mixture of donor:recipient:helper used may be 1:1:1, 2:1:1, 3:1:1, 4:1:1, 5:1:1, 10:1:1, 20:1:1, 50:1:1 or even 100:1:1. In some embodiments, the proportion of the helper strain may be reduced to account for the faster growth rate of E. coli as compared to Methylobacterium and donor:recipient:helper ratios may be 2:2:1, 4:2:1, 6:2:1, 8:2:1, 10:2:1, 20:2:1, 50:2:1, 100:2:1, 5:5:1, 10:5:1, 20:5:1, 50:5:1, 100:5:1, 200:5:1, or 500:5:1. In one embodiment, a AMS-MC plate is used for growth and a helper E. coli strain will not be capable of growing on the AMS-MC. In another embodiment, morphology, including for example white recipient Methylobacterium isolate colonies, can help distinguish in cases where helper strain colonies do grow.

Screening to identify transconjugants is facilitated by the presence of a marker on the mobilizable plasmid as discussed above. Where the marker is an antibiotic resistance gene, the conjugation mixture is plated on media containing the appropriate antibiotic and recipient Methylobacterium isolates that have obtained the mobilizable plasmid as the result of conjugation can be identified. Donor Methylobacterium isolates are also capable of growing in the presence of the antibiotic due to the presence of the marker on the mobilizable plasmid and colony morphology differences can be used to differentiate between the donor and recipient isolates. Additional markers can also be used to facilitate identification of transconjugant Methylobacterium isolates. For example, screenable markers including the fluorescent gene markers described above provide a readily screenable visual identification of Methylobacterium isolates expressing the reporter protein encoded by the marker.

In methods described herein, conjugation rates between the donor Methylobacterium isolate and recipient Methylobacterium isolate in two experiments involving different donor Methylobacterium isolates and the same recipient Methylobacterium isolate, were 1:700 and 1:1300. Thus, the use of multiple methods including antibiotic selection, color reporter markers and colony morphology to facilitate identification of transconjugants may be advantageous.

In some embodiments where the mobilizable plasmid is a native Methylobacterium plasmid and the marker is a genetic sequence, screening and identification methods include colony morphology and DNA detection techniques, including for example qPCR. In such methods, high throughput methods for screening colonies are particularly desirable in order to screen a large number of recipient Methylobacterium isolates to identify transconjugants.

In some embodiments, more than one mobilizable plasmid is transferred from a donor Methylobacterium isolate to a recipient Methylobacterium isolate in the methods described herein. In one embodiment, a recombinant mobilizable plasmid and one or more nativeMethylobacterium mobilizable plasmids are transferred in a conjugation method described herein. In one embodiment, one or more selectable or screenable markers present on the mobilizable plasmid in the donor Methylobacterium isolate may be used to facilitate identification of transconjugants of the recipient Methylobacterium isolate. In one embodiment, the identified recipient Methylobacterium isolates may be further screened using genetic markers specific to plasmids in the donor Methylobacterium isolate to identify transconjugants containing nativeMethylobacterium plasmids transferred during conjugation from the donor Methylobacterium isolate to the recipient Methylobacterium isolate. In one embodiment, 2, 3, 4, 5, or up to 10 native Methylobacterium plasmids are transferred during conjugation from a donor Methylobacterium isolate to a recipient Methylobacterium isolate.

In one embodiment, transformation Methylobacterium isolates are obtained by heat shock, ultra-sound, and/or transduction (e.g. bacteriophage). In another embodiment, transformation Methylobacterium isolates are obtained by electroporation. Electroporation is a method that can be used for direct transformation of bacterial cells with DNA using an electrical field to increase membrane permeability and allow the DNA to be introduced into the cells. In one embodiment, a transformed Methylobacterium isolate is obtained by electroporation of a recipient Methylobacterium isolate with DNA comprising a plasmid having an origin of replication functional in the recipient Methylobacterium isolate and a marker, and screening cells of said recipient Methylobacterium isolate for the presence of a marker to identify a transformed Methylobacterium isolate. In one embodiment electro-competent cells of a recipient Methylobacterium isolate are prepared from cells grown in liquid AMS media. In one embodiment, the media is AMS_GluPP. In one embodiment, cells are grown at 30° C. in a shaker until the culture reaches an OD of from 0.5 to 0.8. In one embodiment, the cells are grown to an OD of 0.6. After the culture reaches the desired OD, the cells are chilled on ice to a temperature of approximately 4° C. and maintained at approximately 4° C. through further harvest and concentration steps.

In one embodiment, electro-competent cells of a recipient Methylobacterium isolate are mixed with DNA containing one or more plasmids of interest. In one embodiment, approximately 50-2000 ng of DNA is added to 50 ul of electro-competent cells. In one embodiment 200 ng-1000 ng of DNA is added with higher amounts resulting in higher transformation rates. In one embodiment, electroporation is conducted using a Gene Pulser, although other commercially available electroporation systems are available and can be used in the methods described herein. In one embodiment, additional chilled media is added to the cell mixture following electroporation and cells are grown for approximately 4 hours to allow for cell recovery. Electroporated cells are pelleted and plated to allow growth and colony production. In one embodiment, colonies of a recipient Methylobacterium isolate will appear within 3 days. In one embodiment, colonies are screened for the presence of a marker on the electroporated plasmid to identify variants of the recipient Methylobacterium isolate. In one embodiment, a marker is a genetic marker. In other embodiments, a marker is a selectable or screenable marker. In some embodiments described herein, multiple markers and/or multiple types of markers may be used in combination. For example, in one embodiment, one or more genetic markers may be used. In other embodiments, one or more genetic markers may be used in combination with one or more selectable markers or screenable markers. In other embodiments, one or more selectable markers may be used, as well as one or more screenable markers, or combination of screenable and selectable markers.

In one embodiment, DNA for electroporation is a foreign plasmid not naturally present in a recipient Methylobacterium isolate. In one embodiment, a foreign plasmid is a recombinant mobilizable plasmid comprising an origin of replication functional in Methylobacterium and a marker. In one embodiment, a foreign plasmid is a native Methylobacterium plasmid. In one embodiment, a foreign plasmid has been identified as encoding one or more genes of interest that confer advantageous traits to a recipient Methylobacterium isolate transformed with the foreign plasmid. In further embodiments, DNA for electroporation may be from a source other than Methylobacterium. In one embodiment, a plasmid for use in electroporation is isolated, or isolated and purified. In other embodiments, DNA for electroporation will contain multiple foreign plasmids. In one embodiment, DNA for electroporation will contain 2, 3, 4, 5, 6, 7, 8, 9, 10, 15, 20, 25 or more plasmids. In one embodiment, where multiple foreign plasmids are employed, the plasmids may be a mixture of native Methylobacterium plasmids obtained from the same Methylobacterium isolates or from different Methylobacterium isolates.

In embodiments provided herein, conjugation or electroporation methods to produce transformed Methylobacterium isolates are used to provide genetically mixed populations of different recipient Methylobacterium isolates containing a variety of foreign plasmids. In one embodiment, the foreign plasmids are native Methylobacterium plasmids. In one embodiment a single donor Methylobacterium isolate may be conjugated to multiple recipient Methylobacterium isolates. In one embodiment multiple donor Methylobacterium isolates may be conjugated to a single Methylobacterium isolate. In one embodiment, multiple donor Methylobacterium isolates may be conjugated to multiple recipient Methylobacterium isolates. In one embodiment, a genetically mixed population of Methylobacterium isolates is produced by electroporation of one or more recipient Methylobacterium isolates. In one embodiment a foreign plasmid is transferred by electroporation into two or more recipient Methylobacterium isolates. In one embodiment, multiple foreign plasmids are transferred by electroporation into a single recipient Methylobacterium isolate. In one embodiment, multiple foreign plasmids are transferred by electroporation into multiple recipient Methylobacterium isolates. In one embodiment, the foreign plasmids are native Methylobacterium plasmids. In one embodiment, foreign plasmids are from bacteria other than Methylobacterium, including but not limited to Rhizobia, Pseudomonas, and Bacillus.

In one embodiment, a recipient Methylobacterium isolate will be transformed by conjugation or electroporation to contain one or more plasmids not naturally present in the recipient Methylobacterium isolate. In one embodiment, a recipient Methylobacterium isolate will contain 2, 3, 4, 5 or up to 10 plasmids not present in the Methylobacterium isolate prior to transformation. In this manner, genetically mixed populations of variant recipient Methylobacterium isolates are obtained which contain foreign plasmids in novel combinations that did not previously exist in any one Methylobacterium isolate or in any particular combination. In one embodiment, the foreign plasmids are native Methylobacterium plasmids. In one embodiment for production of a genetically mixed population of Methylobacterium isolates, multiple genetic, screenable or selectable markers or colony morphology, as described herein, may be used to differentiate donor and transformed recipient Methylobacterium isolates. Such markers may be present on mobilizable plasmids for conjugation into recipient Methylobacterium isolates or on plasmid DNA that for electroporation into recipient Methylobacterium isolates.

Transformed Methylobacterium isolates resulting from the transformation methods described herein, including conjugation and electroporation, will be non-native variant isolates and may exhibit a number of traits that are advantageous for their use in commercial processes. For example, improved growth rates and or altered carbon preferences in transformed Methylobacterium isolates will facilitate their use in industrial applications that benefit from enhanced biomass production, including for example use of the transformed isolates in bioreactors for production of desirable chemical byproducts, including PHA, PHB, carotenoids, and use for harvest of Methylobacterium biomass for use in agriculture or as aquaculture feeds.

In one embodiment for use of transformed Methylobacterium isolates in agriculture, transformed Methylobacterium isolates that have improvements in PGPR genes or pathways, improved colonization rates, improved tolerance to environmental conditions, improved ability to colonize plant surfaces, such as leaves, stems or roots, improved tolerance to desiccation, or improved tolerance to chemicals commonly used in agricultural applications are identified. In one embodiment, a transformed Methylobacterium isolate will have improved biopesticidal activity as exhibited by providing increased protection against one or more plant pests, or will exhibit activity against a broader range of plant pests.

In one embodiment an improved transformed Methylobacterium isolate for use in agricultural applications is identified by screening for useful properties, such as improved tolerance to desiccation, improved activity against plant pests, improved tolerance to chemicals commonly used in agricultural applications, and improved ability to colonize plant surfaces. In one embodiment, the foreign plasmid or plasmids responsible for the improved activity of a transformed Methylobacterium isolate are identified. In one embodiment, a foreign plasmid is isolated and purified and may be used in further electroporation or other direct transformation systems to transform additional recipient Methylobacterium isolates to generate additional improved Methylobacterium isolate.

In some embodiments, transformed Methylobacterium strains are provided herein and function as a delivery system or host to provide colonized plants with nucleic acids, enzymes, and/or encoded proteins to improve plant productivity. In some embodiments, the Methylobacterium is genetically modified to include the nucleic acids, enzymes, and/or encoded proteins. In some embodiments, Methylobacterium is transformed by electroporation or conjugation with plasmids. In some embodiments, Methylobacterium is transformed by insertion of heterologous or recombinant DNA into chromosomal DNA. In some embodiments, the transformed Methylobacterium provide for suppression or silencing of at least one target plant gene or at least one target gene of a plant pest or pathogen. In some embodiments, a transformed Methylobacterium strain is provided to act as a delivery system or host with nucleic acids, enzymes, and/or encoded proteins that are not native to the host Methylobacterium to target at least one gene of a plant pest or pathogen. In some embodiments, induction of an RNAi response directed to endogenous genes in plants or directed to endogenous genes of plant pests or pathogens using transformed Methylobacterium host is provided. In some embodiments, proteins are expressed in a transformed Methylobacterium host and provided to the plant host, including for example pesticidal (e.g., insecticidal, nematicidal, or fungicidal) proteins or proteins that provide for herbicide tolerance. In some embodiments, a transformed Methylobacterium host provides for expression of a combination of gene silencing and/or gene expressing nucleic acids to provide multiple mechanisms for plant improvement. In some embodiments, transformed Methylobacterium are provided which express heterologous nucleic acids that trigger an RNAi response to effect silencing of various target genes in the plants or in the plant pests or pathogens and/or encode pesticidal activity or herbicide tolerance proteins and/or comprise a mutation in an endogenous gene and/or an insertion of a heterologous gene. Methods of using the transformed Methylobacterium to increase crop plant yield, improve plant growth characteristics, provide herbicide tolerance, provide plant quality traits, provide resistance to plant pests and/or pathogens, and to provide other desired effects on the plant are provided, and compositions comprising the Methylobacterium are provided. In some embodiments, plants and seeds are coated with those transformed Methylobacterium. In other embodiments, processed plant products, including but not limited to meals, pastes, and the like, comprise the transformed Methylobacterium.

In certain embodiments of this disclosure, Methylobacterium strains will be transformed to produce high levels of dsRNA molecules and the transformed Methylobacterium can be applied to plants as seed inoculants, soil inoculants (e.g., in furrow applications), and/or as foliar sprays. In certain embodiments, the transformed Methylobacterium can further comprise one or more transgenes that express a pesticidal (e.g., insecticidal, nematicidal, or fungicidal) protein or a herbicide tolerance protein. These bacteria will subsequently colonize the plant and/or regions of the plant (e.g., roots or phylloplane), multiply and amplify the production of dsRNA and/or express the pesticidal or herbicide tolerance proteins. It is anticipated that dsRNA produced by the transformed Methylobacterium on plant surfaces will trigger the RNAi pathway in target organisms (i.e. either the host plant or plant pests including but not limited to insects, plant pathogenic fungi, or nematodes) that will suppress expression of specific plant or plant pest target genes to deliver beneficial effects to the plants. Expression of transgenes for pesticidal or herbicide tolerance proteins will provide further benefits to the plants as the result of such expression.

In some embodiments, the recombinant DNA constructs used to express the nucleic acids that trigger the RNAi response and/or that encode and express a pesticidal protein can be carried in plasmids and/or stably integrated into the chromosome of a host Methylobacterium. In some embodiments, plasmids comprising recombinant DNA constructs for expression of nucleic acids that trigger the RNAi response and/or expression of a pesticidal or herbicide tolerance protein are transformed into a Methylobacterium host by electroporation. In some embodiments a Methylobacterium host is prepared by conjugation between a donor bacterium and a recipient Methylobacterium, whereby one or more plasmids comprising a recombinant DNA construct is transferred to the recipient Methylobacterium to generate a transconjugant Methylobacterium host. In some embodiments, the donor bacterium is a non-Methylobacterium isolate. Non-Methylobacterium isolates that can serve as donor bacterium in the methods provided herein include gram positive bacteria (e.g., a Bacillus sp. including Bacillus thuringiensis) and gram negative bacteria (e.g., Enterobacteriaceae including E. coli). In some embodiments a donor bacterium is E. coli. In some embodiments, a donor bacterium is a Methylobacterium. In some embodiments, Methylobacterium is transformed by tri-parental conjugation and a helper stain is employed. The nucleic acids in the recombinant DNA vectors that can trigger an RNAi response are also referred to herein as “RNAi trigger molecules.” In certain embodiments, the recombinant DNA construct used to express the RNAi trigger molecules and/or that encode and express a pesticidal or herbicide tolerance protein are expressed in a stable, unmarked chromosomal locus of the Methylobacterium. Systems used to insert other heterologous genes in Methylobacterium have been described (Marx and Lidstrom, 2004), and could be used to insert the heterologous recombinant DNA molecules provided herein.

In certain embodiments, the nucleic acids that trigger the RNAi response silence, suppress, or partially suppress a target gene encoded by a plant pest to provide for resistance or tolerance to the plant pest. The target gene can be a gene from a plant pest selected from the group consisting of a nematode, an insect, a plant virus, and a fungus. In general, target genes in the plant pest include endogenous pest genes that are essential for viability, feeding behavior, reproduction, development (e.g., progression from one or more developmental stages to another) survival in the host plant, and/or pathogenesis. Target genes in plant fungi include target genes disclosed in Majumdar et al. (2017). Potential target genes of various pests which could be targeted for RNAi-mediated suppression include insect genes, nematode genes, fungal genes and plant genes.

Non-limiting examples of target insect pests include corn rootworm pests (Diabrotica sp. including virgifera), Colorado potato beetle (CPB, Leptinotarsa decemlineata), red flour beetle (RFB, Tribolium castaneum), European corn borer (ECB, Ostrinia nubilalis), black cutworm (BCW, Agrotis ipsilon), corn earworm (CEW, Helicoverpa zea), fall army worm (FAW, Spodoptera frugiperda), cotton boll weevil (BWV, Anthonomus grandis), velvetbean caterpillar (Anticarsia gemmatalis), soybean looper (Chrysodeixis includens), bean shoot borer (Dectes stem borer), bollworm (Helicoverpa armigera), Western flower thrips (Frankliniella occidentalis), bird cherry-oat aphid (Rhopalosiphum padi), flea beetle (Phyllotreta cruciferae. Phyllotreta striolata. Psylliodes punctulate), and tobacco/onion thrips (Thrips tabaci). Target genes for RNAi-mediated suppression in these and other insect pests include, but are not limited to, chromodomain helicase DNA binding protein 3 (CHD3), beta-tubulin, vacuolar ATP synthase, elongation factor 1-alpha (EFIa), 26S proteosome subunit p28, juvenile hormone epoxide hydrolase, swelling dependent chloride channel (SDCC), glucose-6-phosphate 1-dehydrogenase protein (G6PD), actin (e.g. Act42A), ADP-ribosylation factor 1, transcription factor IIB, chitinase, ubiquitin conjugating enzyme, glyceraldehyde-3-phosphate dehydrogenase (G3PDH), ubiquitin B, juvenile hormone esterase, and alpha tubulin. Methylobacterium that can be transformed for such uses include, but are not limited to Phylloplane, rhizosphere, and/or endosphere colonizers; NLS0020 and variants thereof; NLS0042 and variants thereof; NLS0064 and variants thereof; and NLS0476 and variants thereof. (US20180073037, incorporated herein by reference in its entirety and with respect to target sequences of target insect pests disclosed therein).

Non-limiting examples of target nematode pests include root knot nematodes (Meloidogyne sp.); cyst nematodes (Heterodera sp. and Globodera sp.); and lesion nematodes (Pratylenchus sp.). Target genes for RNAi-mediated suppression in these and other nematode pests include, but are not limited to, FMRFamide (Phe-Met-Arg-Phe) and other FMRFamide-related peptides (FaRPs) 16D10 (root knot nematode secretory peptide) various Heterodera glycines target genes nematode esophageal gland cell proteins. Methylobacterium that can be transformed for such uses include, but are not limited to rhizosphere colonizers; NLS0021 and variants thereof; NLS0037 and variants thereof NLS0038 and variants thereof; NLS0042 and variants thereof; NLS0062 and variants thereof; NLS0069 and variants thereof; NLS0089 and variants thereof; and NLS0934 and variants thereof (US20150361445, incorporated herein by reference in its entirety and with respect to target nematode sequences disclosed therein; Huang et al. (2006); US20160168587, incorporated herein by reference in its entirety and with respect to target nematode sequences disclosed therein; US20100281572, incorporated herein by reference in its entirety and with respect to target nematode sequences disclosed therein).

Non-limiting examples of target fungal pests include Blumeria sp.; Cercospora sp., Colletotrichum sp.; Fusarium sp., Microdochium sp., Pythium. sp.; Phytophora sp., Rhizoctonia sp., Sclerospora sp.; Sclerophthora sp.; Sclerotinia sp.; Septoria sp.; Stenocarpella sp.; and Verticillium sp. Target genes for RNAi-mediated suppression in these and other fungal pests include, but are not limited to, CYP51A, B, C; velvet, chitin synthase, F-box protein required for pathogenicity, Wilt2 (FOW2); OPR; beta-1,3-glucan synthase, hygrophobins 1 (VdH1), cutinase, MAPK, cyclophilin (CYC1), calcineurin (CNB) regulatory subunit. Elicitins, endotoxins pectate lyases, cutin hydrolases. Methylobacterium that can be transformed for such uses include, but are not limited to phylloplane, rhizosphere, and/or endosphere colonizers; NLS0017 and variants thereof; NLS0020 and variants thereof; NLS0064 and variants thereof; NLS0066 and variants thereof, NLS0089 and variants thereof; and NLSO109 and variants thereof (Majumdar et al. (2017) and references cited therein at Table 1; Hane et al. (2014); Lu, T. et al. (2012)).

In certain embodiments, the nucleic acids that trigger the RNAi response silence, suppress, or partially suppress a target gene encoded by the plant. The target plant gene may be either an endogenous plant gene or a heterologous transgene that is introduced into the plant. In certain embodiments, the target plant gene is an herbicide target gene. In certain embodiments, the endogenous herbicide target gene is a 5-enolpyruvylshikimate-3-phosphate synthase (EPSPS), an acetohydroxyacid synthase or an acetolactate synthase (ALS), an acetyl-coenzyme A carboxylase (ACCase), a dihydropteroate synthase, a phytoene desaturase (PDS), a protoporphyrin IX oxygenase (PPO), a hydroxyphenylpyruvate dioxygenase (HPPD), a para-aminobenzoate synthase, a glutamine synthase (GS), a glufosinate-tolerant glutamine synthase, a 1-deoxy-D-xylulose 5-phosphate (DOXP) synthase, a dihydropteroate (DHP) synthase, a phenylalanine ammonia lyase (PAL), a glutathione S-transferase (GST), a D1 protein of photosystem II, a mono-oxygenase, a cytochrome P450, a cellulose synthase, a beta-tubulin, and a serine hydroxymethyltransferase gene. Various sequences that can be used to inhibit herbicide target and other plant genes are disclosed in US Patent Application No. 20110296556 and US Patent Application No. 20130047297, each of which is incorporated herein by reference in its entirety. Inhibition of the herbicide target genes of weeds that have developed herbicide tolerance by Methylobacterium that express nucleic acids that trigger an RNAi response directed against those herbicide target genes can provide for control of those herbicide tolerant weeds. In certain embodiments, the endogenous target plant gene is a gene that can render the plant susceptible to one or more plant pests. Suppression of the endogenous target plant gene by the nucleic acids that trigger the RNAi response can provide for improved tolerance to the pest in comparison to a control plant (e.g., a plant that was not treated or that was mock treated with a Methylobacterium that lacks the recombinant DNA directed to the endogenous plant gene). In certain embodiments, endogenous target plant genes that can be targeted for suppression include plant genes that provide for basic compatibility, host recognition and penetration of the plant pathogen. Examples of such target genes include a MLO (mildew locus O) genes in monocot or dicot plants, where suppression of MLO provides resistance against powdery mildew by disrupting actin-dependent defense pathways (van Schie and Takken (2014). In certain embodiments, endogenous target plant genes that can be targeted for suppression include plant genes that are negative regulators of immune signaling, including ubiquitin ligases PUB22/23/24 and MAPK phosphatases MKP1 and MKP2 (van Schie and Takken (2014). Additional examples of such target genes include: (i) an Arabidopsis Cdd1 (constitutive defense without defect in growth and development 1; Swain et al. (2011) and orthologous genes in crops including dicots such as soybean, Brassica sp., cotton, and the like; or (ii) an Arabidopsis callose synthase Pmr4 gene where suppression increases the salicylic acid defense without decreasing growth (van Schie and Takken (2014) and orthologous genes in crops including dicot crops such as soybean, Brassica sp., cotton, and the like.

In some embodiments, promoters used to drive the expression of the nucleic acid that can trigger an RNAi response can be constitutive. For example, a constitutive promoter can be obtained in certain embodiments by using a portion of a promoter such as the lac promoter, wherein the normal regulatory controls are disengaged by not including the operator sequence (where the Lac repressor would bind) in the partial lac promoter.

In other embodiments, the promoter used to drive the expression of the nucleic acid that can trigger an RNAi response could be a regulated promoter. This would offer the feature, if desired, of having the promoter turned “off” until such time as expression of the nucleic acid molecule that triggers the RNAi response is desired. The turning “on” of the promoter would, in certain embodiments, require the application of an inducer. While the spraying of the treated plants with a chemical inducer may seem problematic, an inducer may be a compound used in standard agronomic practices. In certain embodiments, the application of glyphosate (Roundup) to glyphosate-tolerant crops, could be taken advantage of to turn on a glyphosate-responsive promoter in a Methylobacterium strain that has been transformed to express an RNAi molecule and that has been applied to a plant. In U.S. Pat. No. 8,435,769, which is incorporated herein by reference in its entirety, glyphosate is used to stimulate the accumulation of a small molecule in E. coli. The treatment of the microbial cells with glyphosate derepresses the genes encoding the enzymes of the common aromatic biosynthetic pathway. These include, in E. coli, the first step in the common aromatic biosynthetic pathway which is carried out by three isofunctional DAHP synthase enzymes; these three isofunctional enzymes are encoded by the aroF, aroG, and aroH genes. Similarly, there are two isofunctional enzymes of shikimate kinase, encoded by the aroK and aroL genes. The other enzymes of the pathway consist of single enzymes and are encoded by single genes: the aroB gene encodes 3-dehydroquinate synthase, the aroD gene encodes 3-dehydroquinate dehydratase, the aroE gene encodes shikimate dehydrogenase, the aroA gene encodes EPSP synthase, and the aroC gene encodes chorismate synthase. The promoters for any of these genes could be employed, either taken from their E. coli counterparts, or taken from homologs of these genes that occur in Methylobacterium strains or other microorganisms. Any and all of these glyphosate responsive promoters can be operably linked to a heterologous nucleic acid that triggers an RNAi response directed to a target plant gene.

In some embodiments, the metabolic pathway from chorismate to the downstream metabolites tryptophan, phenylalanine, tyrosine, para-hydroxybenzoic acid, para-aminobenzoic acid, and 2,3-dihydroxybenzoic acid, are carried out by enzymes whose promoters would also be expected to be turned on in response to glyphosate, as the Methylobacterium cell would be starving for these essential metabolites. Tus, for example, in one embodiment the trp promoter could be employed for the purpose of driving the expression of an RNAi trigger molecule in an inducible fashion. Other available promoters for the genes encoding enzymes of these downstream metabolic pathways that can be used include the pheA, tyrB, and tyrA promoters.

In some embodiments, the transformed Methylobacterium express nucleic acid that triggers an RNAi response directed to target genes expressed in insect pests. Without seeking to be limited by theory, it is believed that larval or adult insects feeding on leaves colonized by Methylobacterium cells that are expressing an insecticidal RNAi trigger molecule would swallow the Methylobacterium cells, and through the process of digestion break down the Methylobacterium cells, releasing the trigger molecules which induce an RNAi response that suppresses a gene in the insect, resulting in any of feeding inhibition, stunting, and/or death of the larval or adult insect.

In certain embodiments where the RNAi trigger molecule targets a plant gene, it may be desirable to release the RNAi trigger molecules from the Methylobacterium cells that have colonized leaves. Such release of the RNAi molecules can facilitate uptake of the RNAi molecule for the purpose of altering the phenotype of the plant.

An inducible promoter could also be employed for this purpose, in that the transformed strain of Methylobacterium would have an inducible promoter driving the expression of a heterologous sequence that provides for partial or complete lysis of said Methylobacterium. In certain embodiments, the gene encodes a lytic enzyme that results in lysis of the Methylobacterium cells. Lytic enzymes include, but are not limited to, lysozyme, a 26-kDa peptidoglycan hydrolase of P. aeroginosa (Beveridge, T. (1999) and homologues thereof, autolysins that include N-acetylmuramidases, N-acetylglucosaminidases, N-acetylmuramyl-1-alanine amidases, and endotransglycosidases, and the like. In other embodiments, the lysis gene can be derived or obtained from a bacteriophage that causes lysis of Methylobacterium. Such bacteriophage include, but are not limited to, the bacteriophage deposited as ATCC #PTA-5075 (U.S. Pat. No. 7,550,283, which is incorporated herein by reference in its entirety). This inducible promoter could, in certain embodiments, be a glyphosate inducible promoter. The system could include having the RNAi molecule expressed constitutively, and thus be accumulating inside the Methylobacterium cells, and then lysing the cells by the addition of an inducer, such as glyphosate. In certain embodiments where glyphosate is used as an inducer, the host plant that is colonized by the transformed Methylobacterium strain is a host plant genetically engineered to be resistant to glyphosate.

Yet another consideration is that in certain embodiments, the RNAi trigger molecules are relatively short in length, and it is often the case that short nucleic acids are unstable in bacteria. In certain embodiments, an inducible promoter is employed for both the expression of the RNAi trigger molecule and the lytic enzyme. Without seeking to be limited by theory, it is believed that upon addition of the inducer, there would be a burst of synthesis of the RNAi trigger molecule as the cell wall and membrane are breaking down, with the subsequent release of the RNAi trigger molecules before they are exposed to nucleases. Such nucleases are often compartmentalized in bacteria, and are rendered inactive or ineffective upon cell lysis.

In certain embodiments, expression of dsRNA in Methylobacterium that can function as an RNAi trigger molecule is provided by a bacterial plasmid in which RNA-encoding oligonucleotides directed to a target gene (e.g., having identity and complementarity to the sense or antisense strand of a target gene) are separated by a spacer sequence such that they will form a dsRNA capable of inducing the RNAi response and silencing the target gene. In certain embodiments, these dsRNA molecules that can function as an RNAi trigger molecule will be placed under the control of the strong promoter that is active in Methylobacterium, cloned into a broad host range plasmid and introduced into a Methylobacterium strain by transformation or conjugation. Examples of such strong promoters include, but are not limited to, a M. extorquens methanol dehydrogenase promoter mxaF (McDonald IR and Murrell J C (1997); Marx and Lindstrom (2001)). Examples of such broad host range plasmids include, but are not limited to, pCM80 (Marx and Lindstrom (2001). Spacer sequences that can be positioned between complementary sequences to provide for double stranded hairpin RNAs will range from about 5, 6, 10, 20, 21 to about 50, 100, or 500 nucleotides in length. All chimeric dsRNA-encoding chimeric gene constructs can be verified by sequencing. The pCM80 plasmid containing the dsRNA-encoding sequence can be introduced into a suitable Methylobacterium strain by electroporation and selected onto a medium containing Tetracycline.

Transformed Methylobacterium can be applied to plants, parts thereof, or soil in which the plant is to be grown or where a seed is deposited (e.g., in furrow) to provide resistance to pathogens, herbicides, pests and abiotic stress. Methylobacterium-delivered RNAi technology and/or expression of plant pesticidal or herbicide tolerance proteins can provide commercially useful level of resistance to important pathogens, herbicides, and pests in crops. Methylobacterium can be used as seed treatments, soil treatments (e.g., in furrow applications), and/or foliar sprays. In certain embodiments, the Methylobacterium will multiply and spread on plant tissues and could provide inexpensive and effective control of pests, herbicides, and pathogens though induction of an RNAi response directed against target genes of the pests and/or expression of a pesticidal or herbicide tolerance protein. In certain embodiments, Methylobacterium producing high levels of dsRNA and/or expression of a pesticidal or herbicide tolerance protein can also be killed before being sprayed onto leaves to provide effective control of certain pathogens and pests.

Methylobacterium used in the methods and compositions provided herein, and that can transformed with the recombinant DNA constructs that express an RNAi molecule and/or a pesticidal or herbicide tolerance protein, include Methylobacterium strains that have been subjected to mutagenesis and selected for one or more of a mutation in an endogenous RNAse III gene and/or an improved trait in comparison to a control Methylobacterium strain (e.g., improved desiccation tolerance, improved agricultural chemistry tolerance, improved plant colonization efficiency, improved target plant tissue colonization efficiency, an improved ability to confer pest tolerance to a target plant, and/or an improved ability to elicit a plant defense response in comparison to a control Methylobacterium strain (e.g., the un-mutagenized strain)). In certain embodiments, compositions provided herein can consist of one or more transformed Methylobacterium strains. Methylobacterium can be obtained by various published methods (Madhaiyan et al., 2007) and then subjected to mutagenesis and selected for one or more of a mutation in an endogenous RNAse III gene and/or an improved trait to obtain a Methylobacterium for use in the methods described herein. In certain embodiments, such other Methylobacterium that can be used in mutagenesis and selection experiments to obtain variant Methylobacterium will be Methylobacterium having 16S RNA sequences of at least about 95%, 96%, 97%, 98%, 99% or greater sequence identity to the 16S RNA sequences of other known Methylobacterium. Typing of Methylobacterium by use of 16S RNA sequence comparisons is at least described by Cao et al, 2011.

In certain embodiments, Methylobacterium that can efficiently colonize plants and/or plant parts are subjected to mutagenesis and selected for one or more of a loss-of-function or partial loss-of-function mutation in an endogenous RNAse III gene and/or an improved trait to obtain a host Methylobacterium. Endogenous RNAseIII genes of Methylobacterium that can be targeted for mutagenesis include genes disclosed in Table 4, homologous or orthologous RNAseIII genes having at least 90%, 95%, 98%, or 99% sequence identity across the entire length thereof, genes encoding RNAse III proteins disclosed in Table 4, and genes encoding homologous or orthologous RNAseIII proteins having at least 90%, 95%, 98%, or 99% sequence identity across the entire length of the proteins disclosed in Table 4. Methylobacterium that may colonize plants and/or plant parts are identified, for example, using a colonization screen as described herein. Methylobacterium containing recombinant DNA constructs for expressing RNAi and/or a pesticidal or herbicide tolerance protein, compositions comprising the same, and methods of using the same, including, but not limited to, methods of treating plants or plant part, where the Methylobacterium is a Methylobacterium that can colonize a plant and/or a plant part that is selected from the group consisting of M. extorquens, M. nodulans, M. mesophilicum, M cerasii, M. gossipiicola, Methylobacterium sp. strain LMG6378, M. phyllosphaerae, M. oryzae, M. platani, and M. populi are thus provided. Methods of isolating other Methylobacterium that can colonize plants and/or plant parts have been described herein can also be used.

Representative Methylobacterium strains that can be mutagenized and selected for one or more of a mutation in an endogenous RNAse III gene and/or an improved trait to obtain a host Methylobacterium and then be transformed with the recombinant DNA constructs for expressing RNAi trigger molecules and/or pesticidal or herbicide tolerance protein, compositions comprising the same, and methods of using the same that are provided herein include, but are not limited to, the Methylobacterium of Table 1.

TABLE 1

| Methylobacterium | Depository Accession Numbers for Type Strain |
|---|---|
| Methylobacterium adhaesivum | AR27 = CCM 7305 = CECT 7069 = DSM 17169T = KCTC |
| | 22099T |
| Methylobacterium aerolatum | DSM 19013 = JCM 16406 = KACC 11766 |
| Methylobacterium aminovorans | ATCC 51358 = CIP 105328 = IFO (now NBRC) 15686 = |
| | JCM 8240 = VKM B-2145 |
| Methylobacterium aquaticum | CCM 7218 = CECT 5998 = CIP 108333 = DSM 16371 |
| Methylobacterium brachiatum | DSM 19569 = NBRC 103629 = NCIMB 14379 |
| Methylobacterium bullatum | DSM 21893 = LMG 24788 |
| Methylobacterium cerastii | CCM 7788 = CCUG 60040 = DSM 23679 |
| Methylobacterium chloromethanicum | NCIMB 13688 = VKM B-2223 |
| Methylobacterium | CIP 106787 = DSM 6343 = VKM B-2191 |
| dichloromethanicum | |
| Methylobacterium extorquens | ATCC 43645 = CCUG 2084 = DSM 1337 = IAM 12631 = |
| | IFO (now NBRC) 15687 = JCM 2802 = NCCB 78015 = |
| | NCIB (now NCIMB) 9399 = VKM B-2064. |
| Methylobacterium fujisawaense | ATCC 43884 = CIP 103775 = DSM 5686 = IFO (now |
| | NBRC) 15843 = JCM 10890 = NCIB (now NCIMB) 12417 |
| Methylobacterium gossipiicola | CCM 7572 = NRRL B-51692 |
| Methylobacterium gregans | DSM 19564 = NBRC 103626 = NCIMB 14376 |
| Methylobacterium hispanicum | GP34 = CCM 7219 = CECT 5997 = CIP 108332 = DSM |
| | 16372 |
| Methylobacterium iners | DSM 19015 = JCM 16407 = KACC 11765 |
| Methylobacterium isbiliense | CCM 7304 = CECT 7068 |
| Methylobacterium | Depository Accession Numbers for Type Strain |
| Methylobacterium jeotgali | KCTC 12671 = LMG 23639 |
| Methylobacterium komagatae | DSM 19563 = NBRC 103627 = NCIMB 14377 |
| Methylobacterium longum | CECT 7806 = DSM 23933 |
| Methylobacterium lusitanum | DSM 14457 = NCIMB 13779 = VKM B-2239 |
| Methylobacterium marchantiae | CCUG 56108 = DSM 21328 |
| Methylobacterium mesophilicum | ATCC 29983 = CCUG 16482 = CIP 101129 = DSM 1708 = |
| | ICPB 4095 = IFO (now NBRC) 15688 = JCM 2829 = |
| | LMG 5275 = NCIB (now NCIMB) 11561 = NRRL |
| | B-14246 |
| Methylobacterium nodulans | LMG 21967 = ORS 2060 |
| Methylobacterium organophilum | ATCC 27886 = CIP 101049 = DSM 760 = HAMBI 2263 = |
| | IFO (now NBRC) 15689 = JCM 2833 = LMG 6083 = |
| | NCCB 78041 = VKM B-2066 |
| Methylobacterium oryzae | DSM 18207 = JCM 16405 = KACC 11585 = LMG 23582 |
| Methylobacterium persicinum | DSM 19562 = NBRC 103628 = NCIMB 14378 |
| Methylobacterium phyllosphaerae | DSM 19779 = JCM 16408 = KACC 11716 = LMG 24361 |
| Methylobacterium platani | JCM 14648 = KCTC 12901 |
| Methylobacterium podarium | ATCC BAA-547 = DSM 15083 |
| Methylobacterium populi | ATCC BAA-705 = NCIMB 13946 |
| Methylobacterium radiotolerans | ATCC 27329 = CIP 101128 = DSM 1819 = IFO (now |
| | NBRC) 15690 = JCM 2831 = LMG 2269 = NCIB (now |
| | NCIMB) 10815 = VKM B-2144 |
| Methylobacterium rhodinum | ATCC 14821 = CIP 101127 = DSM 2163 = IFO (now |
| | NBRC) 15691 = JCM 2811 = LMG 2275 = NCIB (now |
| | NCIMB) 9421 = VKM B-2065 |
| Methylobacterium suomiense | DSM 14458 = NCIMB 13778 = VKM B-2238 |
| Methylobacterium tardum | DSM 19566 = NBRC 103632 = NCIMB 14380 |
| Methylobacterium thiocyanatum | ATCC 700647 = DSM 11490 = JCM 10893 = VKM B- |
| | 2197 |
| Methylobacterium variabile | CCM 7281 = CECT 7045 = DSM 16961 |
| Methylobacterium zatmanii | ATCC 43883 = CCUG 36916 = CIP 103774 = DSM 5688 = |
| | IFO (now NBRC) 15845 = JCM 10892 = LMG 6087 = |
| | NCIB (now NCIMB) 12243 = VKM B-2161 |

Depository Key
ATCC: American Type Tissue Culture Collection, Manassas, VA, USA
CCUG: Culture Collection, University of Göteborg, Sweden
CIP: Collection de l'Institut Pasteur, Paris, FR
DSM: DSMZ-German Collection of Microorganisms and Cell Cultures (“DSMZ”), Braunschweig, Germany
JCM: Japan Collection of Microorganisms, Saitama, Japan
LMG: Belgian Co-ordinated Collection of Micro-organisms/Laboratorium voor Microbiologie (“BCCLM”) Ghent, Belgium
NBRC: Biological Resource Center (NBRC),Chiba, Japan
NCIMB: National Collections of Industrial, Food and Marine Bacteria, UK
NRRL: USDA ARS, Peoria, IL., USA

Additional Methylobacterium that can be transformed with the recombinant DNA constructs for expressing RNAi trigger molecules and/or a pesticidal or herbicide tolerance protein to obtain transformed Methylobacterium, compositions comprising the same, and methods of using the same that are provided herein include the Methylobacterium of Table 2, as well as variants thereof, including variants thereof wherein the endogenous RNAse III gene has been mutagenized to introduce a loss-of-function or partial loss of function mutation.

TABLE 2

| | | US Patent, US Patent | |
|---|---|---|---|
| | | Application, or PCT | |
| | | Patent Publication, | |
| | | incorporated herein by | USDA ARS |
| NLS | Origin | reference in its entirety | NRRL No.¹ |
| NLS0017 | Obtained from a | US20180295841 | NRRL B-50931 |
| | peppermint plant grown | US20160295866 | |
| | in Saint Louis County, | | |
| | Missouri, USA | | |
| NLS0020 | Obtained from a horse | US20170238553 | NRRL B-50930 |
| | nettle plant grown in | US20180295841 | |
| | Saint Louis County, | US20160295866 | |
| | Missouri, USA | | |
| NLS0021 | Obtained from a lettuce | US20170164618 | NRRL B-50939 |
| | plant grown in Saint | US20160295866 | |
| | Louis Country, | | |
| | Missouri, USA | | |
| NLS0037 | Obtained from a tomato | USPN 10,098,353 | NRRL B-50941 |
| | plant grown in Saint | | |
| | Louis Country, | | |
| | Missouri, USA | | |
| NLS0038 | Obtained from a tomato | US20170164618 | NRRL B-50942 |
| | plant grown in Saint | | |
| | Louis Country, | | |
| | Missouri, USA | | |
| NLS0042 | Obtained from a | US20170238553 | NRRL B-50932 |
| | soybean plant grown in | US20170164618 | |
| | Saint Louis Country, | US20160295866 | |
| | Missouri, USA | | |
| NLS0062 | | US20170164618 | NRRL B-50937 |
| NLS0064 | Obtained from a corn | US20160302423 | NRRL B-50938 |
| | plant grown in Saint | | |
| | Louis Country, | | |
| | Missouri, USA | | |
| NLS0066 | Obtained from the corn | US20180295841 | NRRL B-50940 |
| | hybrid “MC534” | | |
| | (Masters Choice | | |
| | 3010 State Route 146 | | |
| | East Anna, IL 62906) | | |
| NLS0069 | Obtained from a corn | US20170164618 | NRRL B-50936 |
| | plant grown in Saint | | |
| | Louis Country, | | |
| | Missouri, USA | | |
| NLS0089 | Obtained from a | US20170164618 | NRRL B-50933 |
| | broccoli plant grown in | US20180295841 | |
| | Saint Louis County, | | |
| | Missouri, USA | | |
| NLS0109 | Obtained from a Yucca | US20180295841 | NRRL B-67340 |
| | filamentosa plant in | | |
| | Saint Louis Country, | | |
| | Missouri, USA | | |
| NLS0476 | Obtained from a horse | | Received by the |
| | nettle plant grown in | | NRRL for deposit |
| | Saint Louis County, | | under the Budapest |
| | Missouri, USA | | Treaty as |
| | | | Methylobacterium |
| | | | sp #21 on Jun. |
| | | | 28, 2019 |
| NLS0934 | Obtained from a tomato | US20170164618 | NRRL B-67341 |
| | plant grown in Saint | | |
| | Louis Country, | | |
| | Missouri, USA | | |

¹Deposit number for strain deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Subject to 37 CFR §1.808(b), all restrictions imposed by the depositor on the
availability to the public of the deposited material will be irrevocably removed upon the granting of any patent from this patent application.

Variants of a Methylobacterium strain listed in Table 2 include strains obtained therefrom by genetic transformation, mutagenesis and/or insertion of a heterologous sequence. In some embodiments, such variants are identified by the presence of chromosomal genomic DNA with at least 99%, 99.9, 99.8, 99.7, 99.6%, or 99.5% sequence identity to chromosomal genomic DNA of the strain from which it was derived. Variants of a Methylobacterium strain listed in Table 2 thus include Methylobacterium comprising total genomic DNA (chromosomal and plasmid) with at least 99%, 99.9, 99.8, 99.7, 99.6%, or 99.5% sequence identity to total genomic DNA (chromosomal and plasmid) from the deposited NLS0017, NLS0020, NLS0021, NLS0037, NLS0038, NLS0042, NLS0062, NLS0064, NLS0066, NLS0069, NLS0089, NLS109, and NLS0476 Methylobacterium strains of Table 2. Variants of a Methylobacterium strain listed in Table 2 also include Methylobacterium comprising chromosomal genomic DNA with at least 99%, 99.9, 99.8, 99.7, 99.6%, or 99.5% sequence identity to chromosomal genomic DNA from the deposited NLS0017, NLS0020, NLS0021, NLS0037, NLS0038, NLS0042, NLS0062, NLS0064, NLS0066, NLS0069, NLS0089, NLSO109, and NLS476 Methylobacterium strains of Table 2 In certain embodiments, such variants include or can also be identified by the presence of one or more unique DNA sequences that include: (i) a unique sequence of SEQ ID NO: 1 to 19; (ii) sequences with at least 98% or 99% sequence identity across the full length of SEQ ID NO: 1 to 19.

TABLE 3
Unique sequences associated with Table 2 strains

| | | SEQ |
|---|---|---|
| Strain | Fragment | ID NO |
| NLS0017 | ref4_930 | 1 |
| NLS0017 | ref1_142021 | 2 |
| NLS0017 | ref1_142636 | 3 |
| NLS0020 | ref3_25009 | 4 |
| NLS0020 | ref3_25219 | 5 |
| NLS0020 | ref1_4361220 | 6 |
| NLS0020 | ref1_4602420 | 7 |
| NLS0089 | ref1_194299 | 8 |
| NLS0089 | ref1_194305 | 9 |
| NLS0089 | ref1_194310 | 10 |
| NLS0109 | ref1_135566 | 11 |
| NLS0109 | ref1_135772 | 12 |
| NLS0109 | ref1_169470 | 13 |
| NLS0042 | ref1_86157 | 14 |
| NLS0042 | ref1_142469 | 15 |
| NLS0042 | ref1_142321 | 16 |
| NLS0064 | ref1_153668 | 17 |
| NLS0064 | ref1_3842117 | 18 |
| NLS0064 | ref1_3842278 | 19 |

In certain embodiments, a variant of NLS0017 can comprise a unique sequence having at least 98% or 99% sequence identity across the full length of SEQ ID NO: 1, 2 and/or 3. In certain embodiments, a variant of NLS0020 can comprise a unique sequence having at least 98% or 99% sequence identity across the full length of SEQ ID NO: 4, 5, 6 and/or 7. In certain embodiments, a variant of NLS0089 can comprise a unique sequence having at least 98% or 99% sequence identity across the full length of SEQ ID NO: 8, 9, and/or 10. In certain embodiments, a variant of NLSO109 can comprise a unique sequence having at least 98% or 99% sequence identity across the full length of SEQ ID NO: 11, 12 and/or 13. In certain embodiments, a variant of NLS0042 can comprise a unique sequence having at least 98%, or 99% sequence identity across the full length of SEQ ID NO: 14, 15 and/or 16. In certain embodiments, a variant of NLS0064 can comprise a unique sequence having at least 98% or 99% sequence identity across the full length of SEQ ID NO: 17, 18 and/or 19.

Methylobacterium strains including the strains listed in Table 2 can be mutagenized using random mutagenesis techniques, including radiation and chemical DNA mutagenesis. In certain embodiments, mutations in specific gene targets introduced by random mutagenesis can be identified by Targeting Induced Local Lesions in Genomes (TILLING; Till et al., Genome Res. 2003. 13: 524-530). Alternatively, recombinant DNA based methods may be used to generate a specific mutation, i.e. a point mutation, deletion mutation, or insertion mutation that results in loss of function of the targeted gene. Genome editing techniques can be used, for example, to generate such mutations, including CRISPR/Cas technology, meganucleases, zinc finger nucleases and transcription activator-like effector-based nucleases (TALEN). See, for example, Gaj et al. (2013) (ZFN, TALEN, and CRISPR/Cas-based methods for genome engineering. Trends Biotechnol. 31, 397-405) and Jiang et al. (2015) (Multigene editing in the Escherichia coli genome via the CRISPR-Cas9 system. Appl. Environ. Microbiol. 81, 2506-2514). Other CRISPR-based mutagenesis systems may also be used, including for example, CRISPR-Cpf1 (WO2017015015, incorporated herein by reference in its entirety), CRISPR-Csm1 (U.S. Pat. No. 9,896,696; incorporated herein by reference in its entirety) and other Cas based systems (Makarova et al. (2011) (Unification of Cas protein families and a simple scenario for the origin and evolution of CRISPR-Cas systems. Biol. Direct 6, 38). Other examples of suitable methods include site-directed mutagenesis, oligonucleotide-directed mutagenesis, linker scanning mutagenesis, cassette mutagenesis, and PCR mutagenesis. For description of exemplary random and directed mutagenesis methods, see Directed Mutagenesis: A Practical Approach, MJ McPherson, ed., Oxford University Press, New York (1991) and Molecular Cloning: A Laboratory Manual, Sambrook J, Fritsch E F, Maniatis T M, Cold Spring Harbor Lab Press, Cold Spring Harbor, N.Y., (1989) 2nd Ed.

In certain embodiments, a Methylobacterium strain is an isolated variant that lacks an RNAse III encoding gene, such as NLS0476, or a Methylobacterium strain in which a loss-of-function or partial loss of function mutation is introduced into an endogenous RNAse III encoding gene of the Methylobacterium strain, including a strain listed in Table 1 and Methylobacterium related thereto or a Methylobacterium strain listed in Table 2 and variants thereof. Such loss-of-function mutations include point insertions, deletions, and/or substitutions (e.g., “Indels”) of nucleotides in the gene. A non-limiting example of a loss-of-function mutation include an Indel at the 5′ end of the gene's coding region which introduces a frame shift mutation and/or translation stop codon. Target RNAse III genes that can be subject to mutagenesis include those listed in Table 4 below: (i) SEQ ID NO: 20, SEQ ID NO: 22, SEQ ID NO: 24, SEQ ID NO: 26, SEQ ID NO:28, SEQ ID NO:30, SEQ ID NO:32, SEQ ID NO:34, SEQ ID NO:36, SEQ ID NO:38 and SEQ ID NO:40 in Table 4 or having at least 90%, 95%, 98%, or 99% sequence identity across the full length thereof; (ii) genes encoding the proteins of SEQ ID NO:21, SEQ ID NO: 23, SEQ ID NO: 25, SEQ ID NO: 27, SEQ ID NO:29, SEQ ID NO:31, SEQ ID NO:33, SEQ ID NO:35, SEQ ID NO:37, SEQ ID NO:39 and SEQ ID NO:41 in Table 4 or having at least 90%, 95%, 98%, or 99% sequence identity across the full length thereof. In certain embodiments, the Methylobacterium strain NLS0017, NLS0020, NLS0042, NLS0064, NLS0066, NLS0069, NLS0089, NLSO109, or a variant thereof is subjected to mutagenesis to obtain a host Methylobacterium strain having a loss-of-function or partial loss of function mutation in the endogenous RNAse III gene.

TABLE 4
RNAse III encoding sequences

| Strain and Type | SEQ ID NO |
|---|---|
| NLS0017 RNAse III DNA | 20 |
| NLS0017 RNAse III PRT | 21 |
| NLS0020 RNAse III DNA | 22 |
| NLS0020 RNAse III PRT | 23 |
| NLS0021 RNAse III DNA | 24 |
| NLS0021 RNAse III PRT | 25 |
| NLS0037 RNAse III DNA | 26 |
| NLS0037 RNAse III PRT | 27 |
| NLS0038 RNAse III DNA | 28 |
| NLS0038 RNAse III PRT | 29 |
| NLS0042 RNAse III DNA | 30 |
| NLS0042 RNAse III PRT | 31 |
| NLS0064 RNAse III DNA | 32 |
| NLS0064 RNAse III PRT | 33 |
| NLS0066 RNAse III DNA | 34 |
| NLS0066 RNAse III PRT | 35 |
| NLS0069 RNAse III DNA | 36 |
| NLS0069 RNAse III PRT | 37 |
| NLS0089 RNAse III DNA | 38 |
| NLS0089 RNAse III PRT | 39 |
| NLS0109 RNAse III DNA | 40 |
| NLS0109 RNAse III PRT | 41 |

In certain embodiments, the transformed Methylobacterium provided herein can comprise a recombinant DNA that encodes a pesticidal protein. (e.g., insecticidal, nematocidal, herbicidal or fungicidal) protein. Such pesticidal proteins include proteins that reduce pest viability, feeding behavior, reproduction, development (e.g., progression from one or more developmental stages to another) survival in the host plant, and/or pathogenesis. Insecticidal proteins include vegetative insecticidal proteins (VIP), Cyt toxins (Cyt1A and 2A) and insecticidal endotoxins known as crystal proteins or Cry proteins, including CryIAb, Cry1Ac, CryIF, Cry2Aa, Cry2Ab, Cry3Bb1, Cry34Ab1, Cry35Ab1, Cry3A, and Cry3B (Schepf et al. (1998). In certain embodiments, the Vip3-like gene can be codon optimized, synthesized and cloned in vector pLC291 (Chubiz et al. (2013) for constitutive expression under control of the modified phage PR promoter. The sequence of a codon optimized Vip3-like gene is provided as SEQ ID NO:45.

In certain embodiments, the host Methylobacterium that is transformed to contain recombinant DNA to express an insecticidal protein (e.g., a CRW-inhibitory insecticidal protein) is NLS0020, NLS0042, or a variant thereof. In certain embodiments, the host Methylobacterium that is transformed to contain recombinant DNA to express an insecticidal protein (e.g., a lepidopteran insect-inhibitory insecticidal protein) is NLS0064, NLS0476, or a variant thereof. Antifungal proteins include pathogenesis related proteins (e.g., PR-1 proteins), glucanases, such as (1,3)-glucanases, endoglucanases, chitinases, chitin-binding proteins, an thaumatin-like (TL) proteins, defensins, cyclophilin-like protein, glycine/histidine-rich proteins, ribosome-inactivating proteins (RIPs), lipid-transfer proteins, killer proteins (killer toxins), and protease inhibitors. In certain embodiments, the host Methylobacterium that is transformed to contain recombinant DNA to express an antifungal protein is NLS0017, NLS0020, NLS0064, NLS0066, NLS0062, NLS0089, NLSO109, or a variant thereof. Pesticidal proteins with nematode inhibitory activity include proteinase inhibitors (e.g., cystatins), lectins, certain Bt toxins (Cry5B and Cry6A), and chemodisruptive peptides (e.g., disruptors acetylcholinesterase (AChE) and/or nicotinic acetylcholine receptors). Non-limiting examples of nematode inhibitory proteins are disclosed in Ali et al. (2017). In certain embodiments, the host Methylobacterium that is transformed to contain recombinant DNA to express a nematode inhibitory protein is NLS0021, NLS0037, NLS0038, NLS0042, NLS0062, NLS0069, NLS0089, NLS0934, or a variant thereof. Herbicidal proteins that can be delivered to a plant using genetically modified Methylobacterium as provided herein include 5-enolpyruvylshikimate-3-phosphate synthase (EPSPS), an acetohydroxyacid synthase or an acetolactate synthase (ALS), an acetyl-coenzyme A carboxylase (ACCase), a dihydropteroate synthase, a phytoene desaturase (PDS), a protoporphyrin IX oxygenase (PPO), a hydroxyphenylpyruvate dioxygenase (HPPD), a para-aminobenzoate synthase, a glutamine synthase (GS), a glufosinate-tolerant glutamine synthase, a 1-deoxy-D-xylulose 5-phosphate (DOXP) synthase, a dihydropteroate (DHP) synthase, a phenylalanine ammonia lyase (PAL), a glutathione S-transferase (GST), a D1 protein of photosystem II, a mono-oxygenase, a cytochrome P450, a cellulose synthase, a beta-tubulin, and a serine hydroxymethyltransferase gene. In some embodiments, herbicidal proteins are provided to a transgenic plant that itself expresses the same or a related herbicidal protein. In some embodiments, herbicidal proteins are provided to a transgenic plant that expresses a herbicidal protein providing tolerance to a herbicide other than that targeted by Methylobacterium delivered protein. In some embodiments, herbicidal proteins are provided to a non-transgenic plant that does not express any foreign herbicidal proteins.

In certain embodiments, nucleic sequences for expression of pesticidal proteins are placed under the control of a promoter that is active in Methylobacterium. Examples of such promoters include, but are not limited to, a M. extorquens methanol dehydrogenase promoter mxaF. In certain embodiments, toxins such as Cry, VIP or VIP-like are expressed in Methylobacterium under the control of a mxaF promoter, including, for example Cry1Ac and Vip3L. In some embodiments, a Methylobacterium is modified or transformed to produce insecticidal proteins that are toxic to insect pests of a target host plant. In some embodiments, a Methylobacterium host or delivery system is NLS0064 or NLS0089. In some embodiments, a target host plant is soybean. In some embodiments, a target plant pest is fall armyworm (Spodoptera frugiperda) or lepidopteran.

Methylobacterium containing the recombinant DNA constructs for expressing nucleic acids that can trigger an RNAi response and/or that encode a pesticidal protein can be used to make various compositions useful for treating plants or plant parts. Alternatively, Methylobacterium containing the recombinant DNA constructs for expressing nucleic acids that can trigger an RNAi response, encode a pesticidal protein, and/or compositions comprising the same can be used to treat plants or plant parts. Plants, plant parts, and, in particular, plant seeds that have been at least partially coated with a Methylobacterium containing the recombinant DNA constructs for expressing nucleic acids that can trigger an RNAi response, encode a pesticidal protein, and/or compositions comprising the same are thus provided. Also provided are processed plant products that contain a Methylobacterium containing the recombinant DNA constructs for expressing nucleic acids that can trigger an RNAi response, encode a pesticidal protein, and/or compositions comprising the same. Methylobacterium containing the recombinant DNA constructs for expressing nucleic acids that can trigger an RNAi response, encode a pesticidal protein, and/or compositions comprising the same are particularly useful for treating plant seeds. Seeds that have been at least partially coated with a Methylobacterium containing the recombinant DNA constructs for expressing nucleic acids that can trigger an RNAi response, encode a pesticidal protein, and/or compositions comprising the same are thus provided. Also provided are processed seed products, including, but not limited to, meal, flour, feed, and flakes that contain a Methylobacterium containing the recombinant DNA constructs for expressing nucleic acids that can trigger an RNAi response, encode a pesticidal protein, and/or compositions comprising the same that are provided herein. In certain embodiments, the processed plant product will be non-regenerable (i.e. will be incapable of developing into a plant). In certain embodiments, Methylobacterium containing the recombinant DNA constructs for expressing nucleic acids that can trigger an RNAi response, encode a pesticidal protein, and/or compositions comprising the same will at least partially coat the plant, plant part, or plant seed or that is contained in the processed plant, plant part, or seed product comprises associated Methylobacterium containing the recombinant DNA constructs that can be readily identified by comparing a treated and an untreated plant, plant part, plant seed, or processed product thereof. In certain embodiments, plant pathogenic fungi that are inhibited by the Methylobacterium, compositions comprising the same, plants, plant parts and related methods include a Blumeria sp., a Cercospora sp., a Cochliobolus sp., a Colletotrichum sp., a Diplodia sp., an Erserohilum sp., a Fusarium sp., a Gaeumanomyces sp., a Macrophomina sp., a Magnaporthe sp., a Microdochium sp., a Peronospora sp., a Phakopsora sp., a Phialophora sp., a Phoma sp., a Phymatotrichum sp., a Phytophthora sp., a Pyrenophora sp., a Pyricularia sp, a Pythium sp., a Rhizoctonia sp., a Sclerophthora sp., a Sclerospora sp., a Sclerotium sp., a Sclerotinia sp., a Septoria sp., a Stagonospora sp., a Stenocarpella sp. and a Verticillium sp. In certain embodiments, the insects that are inhibited by the Methylobacterium, compositions comprising the same, plants, plant parts and related methods include Corn Rootworm (Diabrotica sp. including virgifera), Colorado Potato Beetle (CPB, Leptinotarsa decemlineata), Red Flour Beetle (RFB, Tribolium castaneum), European Corn Borer (ECB, Ostrinia nubilalis), Black Cutworm (BCW, Agrotis ipsilon), Corn Earworm (CEW, Helicoverpa zea), Fall Army worm (FAW, Spodoptera frugiperda), Cotton Boll Weevil (BWV, Anthonomus grandis), velvetbean caterpillar (Anticarsia gemmatalis), soybean looper (Chrysodeixis includens), or bean shoot borer (Dectes stem borer) and bollworm (Helicoverpa armigera). In certain embodiments, plant nematodes that are inhibited by the Methylobacterium, compositions comprising the same, plants, plant parts and related methods include a root knot nematode (Meloidogyne sp.), a cyst nematode (e.g., Heterodera sp. or Globodera sp.), or lesion nematode (Pratylenchus sp.). In certain embodiments of the aforementioned methods, the composition is applied to the seed. In certain embodiments, the Pratylenchus sp. is selected from the group consisting of Pratylenchus brachyurus. Pratylenchus coffeae. P. neglectus Pratylenchus penetrans. Pratylenchus scribneri. Pratylenchus thornei. Pratylenchus vulnus, and Pratylenchus zeae. Compositions useful for treating plants or plant parts that comprise Methylobacterium containing the recombinant DNA constructs for expressing nucleic acids that can trigger an RNAi response, encode a pesticidal protein, and/or can also comprise an agriculturally acceptable adjuvant or an agriculturally acceptable excipient. An agriculturally acceptable adjuvant or an agriculturally acceptable excipient is typically an ingredient that does not cause undue phytotoxicity or other adverse effects when exposed to a plant or plant part. In certain embodiments, the emulsion can itself be an agriculturally acceptable adjuvant or an agriculturally acceptable excipient so long as it is not bacteriocidal or bacteriostatic to the Methylobacterium. In other embodiments, the composition further comprises at least one of an agriculturally acceptable adjuvant or an agriculturally acceptable excipient.

Agriculturally acceptable adjuvants used in the compositions include, but are not limited to, components that enhance product efficacy and/or products that enhance ease of product application. Adjuvants that enhance product efficacy can include various wetters/spreaders that promote adhesion to and spreading of the composition on plant parts, stickers that promote adhesion to the plant part, penetrants that can promote contact of the active agent with interior tissues, extenders that increase the half-life of the active agent by inhibiting environmental degradation, and humectants that increase the density or drying time of sprayed compositions. Wetters/spreaders used in the compositions can include, but are not limited to, non-ionic surfactants, anionic surfactants, cationic surfactants, amphoteric surfactants, organo-silicate surfactants, and/or acidified surfactants. Stickers used in the compositions can include, but are not limited to, latex-based substances, terpene/pinolene, and pyrrolidone-based substances. Penetrants can include mineral oil, vegetable oil, esterified vegetable oil, organo-silicate surfactants, and acidified surfactants. Extenders used in the compositions can include, but are not limited to, ammonium sulphate, or menthene-based substances. Humectants used in the compositions can include, but are not limited to, glycerol, propylene glycol, and diethyl glycol. Adjuvants that improve ease of product application include, but are not limited to, acidifying/buffering agents, anti-foaming/de-foaming agents, compatibility agents, drift-reducing agents, dyes, and water conditioners. Anti-foaming/de-foaming agents used in the compositions can include, but are not limited to, dimethopolysiloxane. Compatibility agents used in the compositions can include, but are not limited to, ammonium sulphate. Drift-reducing agents used in the compositions can include, but are not limited to, polyacrylamides, and polysaccharides. Water conditioners used in the compositions can include, but are not limited to, ammonium sulphate.

In certain embodiments, the composition used to treat the seed can contain agriculturally acceptable excipients that include, but are not limited to, woodflours, clays, activated carbon, diatomaceous earth, fine-grain inorganic solids, calcium carbonate and the like. Clays and inorganic solids that can be used with the fermentation broths, fermentation broth products, or compositions provided herein include, but are not limited to, calcium bentonite, kaolin, china clay, talc, perlite, mica, vermiculite, silicas, quartz powder, montmorillonite and mixtures thereof. Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylimide polymers and copolymers, polyhydroxyethyl acrylate, methylacrylimide monomers, alginate, ethylcellulose, polychloroprene and syrups or mixtures thereof. Other useful agriculturally acceptable adjuvants that can promote coating include, but are not limited to, polymers and copolymers of vinyl acetate, polyvinylpyrrolidone-vinyl acetate copolymer and water-soluble waxes. Various surfactants, dispersants, anticaking-agents, foam-control agents, and dyes disclosed herein and in U.S. Pat. No. 8,181,388 can be adapted for use with an active agent comprising the transformed Methylobacterium, such as those containing recombinant DNA constructs for expressing nucleic acids that can trigger an RNAi response, or encode a pesticidal protein, and/or compositions containing the same that are provided herein.

In some embodiments, the composition or method disclosed herein may comprise one or more additional components. In some embodiments a second component can be an additional active ingredient, for example, a pesticide or a second biological. The pesticide may be, for example, an insecticide, a fungicide, an herbicide, or a nematicide. The second biological can be a biocontrol microbe.

Non-limiting examples of insecticides and nematicides include carbamates, diamides, macrocyclic lactones, neonicotinoids, organophosphates, phenylpyrazoles, pyrethrins, spinosyns, synthetic pyrethroids, tetronic and tetramic acids. In particular embodiments insecticides and nematicides include abamectin, aldicarb, aldoxycarb, bifenthrin, carbofuran, chlorantraniliporle, chlothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodichlofen, spirotetramat, tefluthrin, thiacloprid, thiamethoxam, and thiodicarb,

Non-limiting examples of useful fungicides include aromatic hydrocarbons, benzimidazoles, benzthiadiazole, carboxamides, carboxylic acid amides, morpholines, phenylamides, phosphonates, quinone outside inhibitors (e.g. strobilurins), thiazolidines, thiophanates, thiophene carboxamides, and triazoles. Particular examples of fungicides include acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole.

Non-limiting examples of herbicides include ACCase inhibitors, acetanilides, AHAS inhibitors, carotenoid biosynthesis inhibitors, EPSPS inhibitors, glutamine synthetase inhibitors, PPO inhibitors, PS II inhibitors, and synthetic auxins, Particular examples of herbicides include acetochlor, clethodim, dicamba, flumioxazin, fomesafen, glyphosate, glufosinate, mesotrione, quizalofop, saflufenacil, sulcotrione, and 2,4-D.

In some embodiments, the compositions or methods disclosed herein may comprise an additional active ingredient which may be a second biological. The second biological could be a biological control agent, other beneficial microorganisms, microbial extracts, natural products, plant growth activators or plant defense agent. Non-limiting examples of biological control agents include bacteria, fungi, beneficial nematodes, and viruses.

In certain embodiments, the second biological can be Methylobacterium selected from the group consisting of ISO01 (NRRL B-50929), ISO02 (NRRL B-50930), ISO03 (NRRL B-50931), ISO04 (NRRL B-50932), ISO05 (NRRL B-50933), ISO06 (NRRL B-50934), ISO07 (NRRL B-50935), ISO08 (NRRL B-50936), ISO09 (NRRL B-50937), ISO10 (NRRL B-50938), ISO11 (NRRL B-50939), ISO12 (NRRL B-50940), ISO13 (NRRL B-50941), and ISO14 (NRRL B-50942). In certain embodiments, the second biological can be a Methylobacterium having chromosomal genomic DNA with at least 99%, 99.9, 99.8, 99.7, 99.6%, or 99.5% sequence identity to chromosomal genomic DNA of ISO01 (NRRL B-50929), ISO02 (NRRL B-50930), ISO03 (NRRL B-50931), ISO04 (NRRL B-50932), ISO05 (NRRL B-50933), ISO06 (NRRL B-50934), ISO07 (NRRL B-50935), ISO08 (NRRL B-50936), ISO09 (NRRL B-50937), ISO10 (NRRL B-50938), ISO1 (NRRL B-50939), ISO12 (NRRL B-50940), ISO13 (NRRL B-50941), or ISO14 (NRRL B-50942).

In certain embodiments, the fermentation broth, fermentation broth product, or compositions that comprise transformed Methylobacterium can further comprise one or more introduced additional active ingredients or microorganisms of pre-determined identity other than Methylobacterium. In certain embodiments, the second biological can be a bacterium of the genus Actinomycetes, Agrobacterium, Arthrobacter, Alcaligenes, Aureobacterium, Azobacter, Beijerinckia, Brevibacillus, Burkholderia, Chromobacterium, Clostridium, Clavibacter, Comomonas, Corynebacterium, Curtobacterium, Enterobacter Flavobacterium, Gluconobacter, Hydrogenophage, Klebsiella, Paenibacillus, Pasteuria, Phingobacterium, Photorhabdus, Phyllobacterium, Pseudomonas, Rhizobium, Bradyrhizobium, Serratia, Stenotrophomonas, Variovorax, and Xenorhadbus. In particular embodiments the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens.

In certain embodiments the second biological can be a fungus of the genus Alternaria, Ampelomyces, Aspergillus, Aureobasidium, Beauveria, Colletotrichum, Coniothyrium, Gliocladium, Metarhisium, Muscodor, Paecilonyces, Trichoderma, Typhula, Ulocladium, and Verticilium. In particular embodiments the fungus is Beauveria bassiana. Coniothyrium minitans. Gliocladium vixens. Muscodor albus. Paecilomyces lilacinus, or Trichoderma polysporum.

In further embodiments the second biological can be a plant growth activator or plant defense agent including, but not limited to harpin, Reynoutria sachalinensis, jasmonate, lipochitooligosaccharides, and isoflavones.

In further embodiments, the second biological can include, but is not limited to, various Bacillus sp., Pseudomonas sp., Coniothyrium sp., Pantoea sp., Streptomyces sp., and Trichoderma sp. Microbial biopesticides can be a bacterium, fungus, virus, or protozoan. Particularly useful biopesticidal microorganisms include various Bacillus subtilis, Bacillus thuringiensis, Bacillus pumilis, Pseudomonas syringae, Trichoderma harzianum, Trichoderma virens, and Streptomyces lydicus strains. Other microorganisms that are added can be genetically engineered or naturally occurring isolates that are available as pure cultures. In certain embodiments, it is anticipated that the bacterial or fungal microorganism can be provided in the fermentation broth, fermentation broth product, or composition in the form of a spore.

Methods of treating plants and/or plant parts with transformed Methylobacterium, and compositions containing the same are also provided herein. Treated plants, and treated plant parts obtained therefrom, include, but are not limited to, corn, soybean, Brassica sp. (e.g., B. napus, B. rapa, B. juncea), alfalfa, rice, rye, wheat, barley, oats, sorghum, millet (e.g., pearl millet (Pennisetum glaucum)), proso millet (Panicum miliaceum), foxtail millet (Setaria italica), finger millet (Eleusine coracana), sunflower, safflower, tobacco, potato, peanuts, cotton, sweet potato (Ipomoea batatus), cassava, coffee, coconut, pineapple, citrus trees, cocoa, tea, date palm, banana, apple, pear, grape, berry plants (including, but not limited to blackberry, raspberry, strawberry or blueberry plants), avocado, fig, guava, kiwi, mango, olive, papaya, cashew, macadamia, almond, sugar beets, sugarcane, tomatoes, peppers, lettuce, green beans, lima beans, peas, lentils, cucurbits (including, but not limited to cucumber, cantaloupe, melons, squash, pumpkin, and zucchini). In other embodiments, treated plants include ornamentals (including, but not limited to, azalea, hydrangea, hibiscus, roses, tulips, daffodils, petunias, carnation, poinsettia, and chrysanthemum), conifers (including, but not limited to pines such as loblolly pine, slash pine, ponderosa pine, lodge pole pine, and Monterey pine; Douglas-fir; Western hemlock; Sitka spruce; redwood; true firs such as silver fir and balsam fir; and cedars such as Western red cedar and Alaska yellow-cedar) and turfgrass (including, but are not limited to, annual bluegrass, annual ryegrass, Canada bluegrass, fescue, bentgrass, wheatgrass, Kentucky bluegrass, orchard grass, ryegrass, redtop, Bermuda grass, St. Augustine grass, and zoysia grass).

In certain embodiments where the transformed Methylobacterium expresses an RNAi molecule that targets an endogenous plant gene, the plant gene will be from the target plant. In certain embodiments, the target plant is one of the aforementioned plants. Seeds or other propagules of any of the aforementioned plants can be treated with the Methylobacterium containing the recombinant DNA constructs for expressing nucleic acids that can trigger an RNAi response, encode a pesticidal protein, and compositions containing the same provided herein.

In certain embodiments, plants and/or plant parts are treated by applying transformed Methylobacterium such as those containing the recombinant DNA constructs for expressing nucleic acids that can trigger an RNAi response, or encode a pesticidal protein, and compositions containing the same as a spray. Such spray applications include, but are not limited to, treatments of a single plant part or any combination of plant parts. Spraying can be achieved with any device that will distribute the compositions comprising the Methylobacterium to the plant and/or plant part(s). Useful spray devices include a boom sprayer, a hand or backpack sprayer, crop dusters (i.e. aerial spraying), and the like. Spraying devices and or methods providing for application of transformed Methylobacterium, such as those containing the recombinant DNA constructs for expressing nucleic acids that can trigger an RNAi response, encode a pesticidal protein, and/or compositions containing the same to either one or both of the adaxial surface and/or abaxial surface can also be used. Plants and/or plant parts that are at least partially coated with transformed Methylobacterium, such as those containing recombinant DNA constructs for expressing nucleic acids that can trigger an RNAi response, or encode a pesticidal protein, and compositions containing the same are also provided herein. Such plant parts include seeds, leaves, roots, stems, tubers, flowers, and fruit. Also provided herein are processed plant products that comprise transformed Methylobacterium, and compositions containing the same.

In certain embodiments, seeds are treated by exposing the seeds to the transformed Methylobacterium such as those containing recombinant DNA constructs for expressing nucleic acids that can trigger an RNAi response, or encode a pesticidal protein, and/or compositions containing the same that are provided herein. Seeds can be treated with transformed Methylobacterium provided herein by methods including, but not limited to, imbibition, coating, spraying, and the like. Seed treatments can be effected with both continuous and/or a batch seed treaters. In certain embodiments, the coated seeds may be prepared by slurrying seeds with a coating composition containing transformed Methylobacterium, such as those containing recombinant DNA constructs for expressing nucleic acids that can trigger an RNAi response, or encode a pesticidal protein, and/or compositions containing the same and air drying the resulting product. Air drying can be accomplished at any temperature that is not deleterious to the seed or the Methylobacterium, but will typically not be greater than 30 degrees Centigrade. The proportion of coating that comprises a transformed Methylobacterium, and/or compositions containing the same includes, but is not limited to, a range of 0.1 to 25% by weight of the seed, 0.5 to 5% by weight of the seed, and 0.5 to 2.5% by weight of seed. Various seed treatment compositions and methods for seed treatment disclosed in U.S. Pat. Nos. 5,106,648, 5,512,069, and 8,181,388 are incorporated herein by reference in their entireties and can be adapted for use with an active agent comprising the transformed Methylobacterium or compositions provided herein.

Also provided herein are methods of identifying Methylobacterium, variants of the Methylobacterium and compositions including, but not limited to, soil samples, plant parts, including plant seeds, or processed plant products, comprising or coated with Methylobacterium sp. by assaying for the presence of nucleic acid fragments comprising at least 40, 50, 60, 100, 120, 180, 200, 240, or 300 nucleotides of SEQ ID NO: in the Methylobacterium or compositions. In certain embodiments, such methods can comprise subjecting a sample suspected of containing Methylobacterium sp. NLS0042, NLS0064, or a variant thereof to a nucleic acid analysis technique and determining that the sample contains one or more nucleic acid containing a sequence of at least about 50, 100, 200, or 300 nucleotides that is identical to a contiguous sequence in SEQ ID NO: 14, 15, 16, 17, 18, or 19, wherein the presence of a sequence that is identical to a contiguous sequence in SEQ ID NO: 14, 15, or 16 is indicative of the presence of NLS0042 or a variant thereof in the sample and wherein the presence of a sequence that is identical to a contiguous sequence in SEQ ID NO: 17, 18, or 19 is indicative of the presence of NLS0064 or a variant thereof in the sample. Such nucleic acid analyses include, but are not limited to, techniques based on nucleic acid hybridization, polymerase chain reactions, mass spectroscopy, nanopore based detection, branched DNA analyses, combinations thereof, and the like. One example of such a nucleic acid analysis is a qPCR Locked Nucleic Acid (LNA) based assay. Such analysis can be used to detect Methylobacterium strains present at a concentration (CFU/gm of sample) of 10¹, 10⁴, 10⁵, 10⁶ or more. In certain embodiments, any of the aforementioned detection methods can comprise the steps of: (i) contacting the sample with a DNA primer pair, wherein said primer pair comprises forward and reverse primers for amplification of a DNA fragment comprising or located within SEQ ID NO:14, 15, 16, 17, 18, or 19, thereby generating a DNA fragment, (ii) contacting said DNA fragment with a probe specific for the presence of said DNA fragment, and (iii) comparing the results of said contacting with positive and negative controls to determine the presence of the sequence indicative of NLS0042 and NLS0064 in said sample. In certain embodiments, the sample is a plant material that was treated with one or more of Methylobacterium strains selected from NLS0042, NLS0064, or a variant thereof. In certain embodiments, the plant material in the sample is comprised of leaves, roots, and/or seeds. In certain embodiments, the plant material is a processed plant product from a plant treated with one or more Methylobacterium strains selected from NLS0042 or NLS0064. In certain embodiments, the sample is a soil sample.

### EMBODIMENTS

Various embodiments of the Methylobacterium, compositions, and methods provided herein are included in the following non-limiting lists.

### Embodiment List One

1. A Methylobacterium comprising a recombinant DNA construct wherein a promoter is operably linked to a heterologous sequence encoding a nucleic acid that can trigger an RNAi response.
   2. The Methylobacterium of embodiment 1, wherein said RNAi response inhibits expression of a target plant pathogen gene.
   3. The Methylobacterium of embodiment 1, wherein said Methylobacterium further comprises a recombinant DNA construct wherein a promoter is operably linked to a heterologous sequence comprising a nucleic acid that encodes a pesticidal protein.
   4. The Methylobacterium of embodiment 1, wherein said RNAi response inhibits expression of a target plant gene.
   5. The Methylobacterium of embodiment 1, wherein said promoter is an inducible promoter.
   6. The Methylobacterium of embodiment 5, wherein said inducible promoter is a glyphosate inducible promoter.
   7. The Methylobacterium of embodiment 6, wherein said glyphosate inducible promoter is selected from the group consisting of an trp, pheA, tyrA, tyrB, aroA, aroB, aroC, aroD, aroE, aroF, aroG, aroH, aroK, and an aroL promoter.
   8. The Methylobacterium of any one of embodiments 1-7, wherein said Methylobacterium further comprises a recombinant DNA construct wherein an inducible promoter is operably linked to a heterologous sequence that provides for partial or complete lysis of said Methylobacterium upon exposure to an agent that induces the promoter.
   9. The Methylobacterium of embodiment 8, wherein said inducible promoter that is operably linked to a heterologous sequence that provides for partial or complete lysis of said Methylobacterium is a glyphosate inducible promoter.
   10. The Methylobacterium of embodiment 9, wherein said glyphosate inducible promoter that is operably linked to a heterologous sequence that provides for partial or complete lysis of said Methylobacterium is selected from the group consisting of an trp, pheA, tyrA, tyrB, aroA, aroB, aroC, aroD, aroE, aroF, aroG, aroH, aroK, and an aroL promoter.
   11. The Methylobacterium of embodiment 8, wherein said heterologous sequence that provides for partial or complete lysis of said Methylobacterium encodes an enzyme selected from the group consisting of lysozyme, a 26 kD peptidoglycan hydrolase, an N-acetylmuramidase, an N-acetylglucosaminidase, an N-acetylmuramyl-1-alanine amidases, and an endotransglycosidase.
   12. A composition comprising the Methylobacterium of any one of embodiments 1-11 and at least one agriculturally acceptable excipient or adjuvant.
   13. An engineered Methylobacterium strain that comprises:
   i) a first recombinant DNA construct wherein a promoter is operably linked to at least one heterologous sequence encoding a nucleic acid that can trigger an RNAi response, and
   ii) a second recombinant DNA construct wherein a promoter is operably linked to a heterologous sequence comprising a nucleic acid that encodes a pesticidal protein; wherein a selected Methylobacterium strain comprises the first and second recombinant DNA construct.
   14. The Methylobacterium of embodiment 13, wherein said RNAi response inhibits expression of a target plant pest gene and wherein said pesticidal protein is active against said target plant pest.
   15. The Methylobacterium of embodiment 14 wherein said target plant pest is an insect pest or a pest that causes a plant disease.
   16. The Methylobacterium of embodiment 15 wherein said insect pest is a Coleopteran, Lepidopteran and/or Hemipteran species pest.
   17. The Methylobacterium of embodiment 15 wherein said pest that causes a plant disease is a fungus, bacteria, virus and/or nematode pest.
   18. The Methylobacterium of embodiment 13, wherein said RNAi response inhibits expression of a gene in a first target plant pest and wherein said pesticidal protein is active against a second target plant pest.
   19. The Methylobacterium of embodiment 18 wherein said first and second target plant pests are insect pests.
   20. The Methylobacterium of embodiment 19, wherein said insect pests are Coleopteran, Lepidopteran and/or Hemipteran species pests.
   21. The Methylobacterium of embodiment 18 wherein said first and second target plant pests are pests that cause a plant disease.
   22. The Methylobacterium of embodiment 21 wherein said pests that cause a plant disease are fungi, bacteria, virus and/or nematode pests.
   23. The engineered Methylobacterium strain of any one of embodiments 13-22, wherein said selected Methylobacterium strain is selected based on performance in desiccation tolerance, agricultural chemistry tolerance, and colonization efficiency screens.
   24. The engineered Methylobacterium strain of embodiment 23, wherein said selected Methylobacterium strain is an effective colonizer of a plant shoot.
   25. The engineered Methylobacterium strain of embodiment 24, wherein said plant is soy and said Methylobacterium strain is NLS0064 or a variant thereof.
   26. The engineered Methylobacterium strain of embodiment 23, wherein said Methylobacterium strain is an effective colonizer of plant roots.
   27. The engineered Methylobacterium strain of embodiment 26, wherein said plant is corn and said Methylobacterium strain is NLS0042 or a variant thereof.
   28. The engineered Methylobacterium strain of any one of embodiments 13-22, wherein said selected Methylobacterium strain is a mutant strain lacking RNAse III activity.
   29. The engineered Methylobacterium strain of embodiment 28 wherein said Methylobacterium strain is NLS0476 or a variant thereof.
   30. A composition comprising the Methylobacterium of any one of embodiments 13-22, and at least one agriculturally acceptable excipient or adjuvant.
   31. A method of altering a phenotypic trait in a host plant comprising the step of applying the Methylobacterium of any one of embodiments 1-11 or the engineered Methylobacterium of any one of embodiments 13-22 to a plant or a plant part.
   32. The method of embodiment 31, wherein said plant part is a seed.
   33. The method of embodiment 31 or 32, wherein the alteration in the phenotypic trait is increased in comparison to a control plant to which a Methylobacterium lacking a recombinant DNA construct had been applied.
   34. A method of altering a phenotypic trait in a host plant comprising the step of applying the composition of embodiment 10 or 30, to a plant or a plant part.
   35. The method of embodiment 34, wherein said plant part is a seed.
   36. A method for inhibiting a plant pest in a host plant comprising the step of applying the Methylobacterium of any one of embodiments 1-11 or the engineered Methylobacterium of any one of embodiments 13-22 to a plant, a plant part, and/or to soil in which the plant will be grown or plant part deposited.
   37. The method of embodiment 36, wherein said plant part is a seed.
   38. The method of embodiment 36 or 37, wherein the inhibition of the plant pathogen is increased in comparison to a control plant to which a Methylobacterium lacking a recombinant DNA construct had been applied.
   39. A method for inhibiting a plant pathogen in a host plant comprising the step of applying the composition of embodiment 10 or 30 to a plant, a plant part, and/or to soil in which the plant will be grown or plant part deposited.
   40. The method of embodiment 39, wherein said plant part is a seed.
   41. The method of embodiment 39 or 40, wherein the inhibition of the plant pathogen is increased in comparison to a control plant to which a composition containing Methylobacterium lacking a recombinant DNA construct had been applied.

### Embodiment List 2

1. A method of producing a transconjugant Methylobacterium isolate, comprising: incubating (i) a donor Methylobacterium isolate comprising a mobilizable plasmid containing a marker; and (ii) a recipient Methylobacterium isolate; wherein the mobilizable plasmid has an origin of replication functional in the recipient Methylobacterium isolate; wherein said mobilizable plasmid is transferred from said donor Methylobacterium isolate to said recipient Methylobacterium isolate; and screening cells of said recipient Methylobacterium isolate for the presence of the mobilizable plasmid marker to identify a transconjugant Methylobacterium isolate.
2. The method of embodiment 1 wherein said marker is a selectable marker.
3. The method of embodiment 2 where said selectable marker is a gene encoding resistance to an antibiotic.
4. The method of embodiment 1 wherein said marker is a genetic sequence marker.
5. The method of embodiment 1 wherein said marker is a screenable marker.
6. the method of embodiment 5 wherein said screenable marker encodes a fluorescent protein.
7. The method of embodiment 1 wherein said mobilizable plasmid is a native Methylobacterium plasmid.
8. The method of embodiment 1 wherein said method further comprises the use of a helper strain, wherein said helper strain encodes conjugation transfer functions.
9. The method of embodiment 1 wherein said recipient Methylobacterium isolate contains a mutation in the carotenoid biosynthesis pathway.
10. The method of embodiment 9 wherein said mutation results in loss of function of crtI.
11. The method of embodiment 1 wherein said origin of replication is an RK2 origin of replication.
12. A method of producing a population of transconjugant Methylobacterium isolates, comprising the steps of
(i) incubating a composition comprising a first donor Methylobacterium isolate comprising a mobilizable plasmid containing an origin of replication functional in Methylobacterium and a marker, and one or more recipient Methylobacterium isolates under conditions wherein said mobilizable plasmid is transferred from said donor Methylobacterium isolate to said recipient Methylobacterium isolate or isolates; and
(ii) screening cells of said recipient Methylobacterium isolate or isolates for the presence of the mobilizable plasmid marker to identify one or more transconjugant Methylobacterium isolates.
13. The method of embodiment 12 wherein said marker is a selectable marker or screenable marker.
14. The method of embodiment 12 wherein said composition comprises a one or more additional donor Methylobacterium isolates comprising a mobilizable plasmid containing an origin of replication functional in Methylobacterium and a marker.
15. The method of embodiment 14, wherein the marker on the mobilizable plasmid in said first donor Methylobacterium isolate is the same marker as on the mobilizable plasmid in said one or more additional Methylobacterium isolates.
16. The method of embodiment 15 where the marker on the mobilizable plasmid in said first donor Methylobacterium isolate is a different marker than the marker on the mobilizable plasmid in said one or more additional donor Methylobacterium isolates.
17. The method of embodiment 14 wherein the mobilizable plasmids of said first and additional donor Methylobacterium isolates each comprise a different marker.
18. A method of producing a transformed Methylobacterium isolate, comprising: transforming a recipient Methylobacterium isolate with a plasmid having an origin of replication functional in the recipient Methylobacterium isolate and a marker; wherein said plasmid is transferred to said recipient Methylobacterium isolate; and screening cells of said recipient Methylobacterium isolate for the presence of the marker to identify a transformed Methylobacterium isolate.
19. The method of embodiment 18 wherein said marker is a genetic sequence marker.
20. The method of embodiment 18 wherein said plasmid is a native Methylobacterium plasmid.
21. The method of embodiment 18 wherein transforming is selected from the group consisting of electroporation, heat shock, ultra-sound, and transduction.

### Embodiment List Three

1. A Methylobacterium comprising a recombinant DNA construct wherein a promoter is operably linked to a heterologous sequence encoding a nucleic acid that can trigger an RNAi response.
2. The Methylobacterium of embodiment 1, wherein said RNAi response inhibits expression of a target plant pathogen gene.
3. The Methylobacterium of embodiment 1, wherein said Methylobacterium further comprises a recombinant DNA construct wherein a promoter is operably linked to a heterologous sequence comprising a nucleic acid that encodes a pesticidal or herbicide tolerance protein.
4. The Methylobacterium of embodiment 1, wherein said RNAi response inhibits expression of a target plant gene.
5. The Methylobacterium of embodiment 1, wherein said promoter is an inducible promoter.
6. The Methylobacterium of embodiment 5, wherein said inducible promoter is a glyphosate inducible promoter.
7. The Methylobacterium of embodiment 6, wherein said glyphosate inducible promoter is selected from the group consisting of an trp, pheA, tyrA, tyrB, aroA, aroB, aroC, aroD, aroE, aroF, aroG, aroH, aroK, and an aroL promoter.
8. The Methylobacterium of any one of embodiments 1-7, wherein said Methylobacterium further comprises a recombinant DNA construct wherein an inducible promoter is operably linked to a heterologous sequence that provides for partial or complete lysis of said Methylobacterium upon exposure to an agent that induces the promoter.
9. The Methylobacterium of embodiment 8, wherein said inducible promoter that is operably linked to a heterologous sequence that provides for partial or complete lysis of said Methylobacterium is a glyphosate inducible promoter.
10. The Methylobacterium of embodiment 9, wherein said glyphosate inducible promoter that is operably linked to a heterologous sequence that provides for partial or complete lysis of said Methylobacterium is selected from the group consisting of an trp, pheA, tyrA, tyrB, aroA, aroB, aroC, aroD, aroE, aroF, aroG, aroH, aroK, and an aroL promoter.
11. The Methylobacterium of embodiment 8, wherein said heterologous sequence that provides for partial or complete lysis of said Methylobacterium encodes an enzyme selected from the group consisting of lysozyme, a 26 kD peptidoglycan hydrolase, an N-acetylmuramidase, an N-acetylglucosaminidase, an N-acetylmuramyl-1-alanine amidases, and an endotransglycosidase.
12. A composition comprising the Methylobacterium of any one of embodiments 1-11 and at least one agriculturally acceptable excipient or adjuvant.
13. An engineered Methylobacterium strain that comprises:
i) a first recombinant DNA construct wherein a promoter is operably linked to at least one heterologous sequence encoding a nucleic acid that can trigger an RNAi response, and
ii) a second recombinant DNA construct wherein a promoter is operably linked to a heterologous sequence comprising a nucleic acid that encodes a pesticidal or herbicide tolerance protein; wherein a selected Methylobacterium strain comprises the first and second recombinant DNA construct.
14. The Methylobacterium of embodiment 13, wherein said RNAi response inhibits expression of a target plant pest gene and wherein said pesticidal protein is active against said target plant pest.
15. The Methylobacterium of embodiment 14 wherein said target plant pest is an insect pest or a pest that causes a plant disease.
16. The Methylobacterium of embodiment 15 wherein said insect pest is a Coleopteran, Lepidopteran and/or Hemipteran species pest.
17. The Methylobacterium of embodiment 15 wherein said pest that causes a plant disease is a fungus, bacteria, virus and/or nematode pest.
18. The Methylobacterium of embodiment 13, wherein said RNAi response inhibits expression of a gene in a first target plant pest and wherein said pesticidal protein is active against a second target plant pest.
19. The Methylobacterium of embodiment 18 wherein said first and second target plant pests are insect pests.
20. The Methylobacterium of embodiment 19, wherein said insect pests are Coleopteran, Lepidopteran and/or Hemipteran species pests.
21. The Methylobacterium of embodiment 18 wherein said first and second target plant pests are pests that cause a plant disease.
22. The Methylobacterium of embodiment 21 wherein said pests that cause a plant disease are fungi, bacteria, virus and/or nematode pests.
23. The engineered Methylobacterium strain of any one of embodiments 13-22, wherein said selected Methylobacterium strain is selected based on performance in desiccation tolerance, agricultural chemistry tolerance, and colonization efficiency screens.
24. The engineered Methylobacterium strain of embodiment 23, wherein said selected Methylobacterium strain is an effective colonizer of a plant shoot.
25. The engineered Methylobacterium strain of embodiment 24, wherein said plant is soy and said Methylobacterium strain is NLS0064 or a variant thereof.
26. The engineered Methylobacterium strain of embodiment 23, wherein said Methylobacterium strain is an effective colonizer of plant roots.
27. The engineered Methylobacterium strain of embodiment 26, wherein said plant is corn and said Methylobacterium strain is NLS0042 or a variant thereof.
28. The engineered Methylobacterium strain of any one of embodiments 13-22, wherein said selected Methylobacterium strain is a mutant strain lacking RNAse III activity.
29. The engineered Methylobacterium strain of embodiment 28 wherein said Methylobacterium strain is NLS0476 or a variant thereof.
30. A composition comprising the Methylobacterium of any one of embodiments 13-22, and at least one agriculturally acceptable excipient or adjuvant.
31. A method of altering a phenotypic trait in a host plant comprising the step of applying the Methylobacterium of any one of embodiments 1-11 or the engineered Methylobacterium of any one of embodiments 13-22 to a plant or a plant part.
32. The method of embodiment 31, wherein said plant part is a seed.
33. The method of embodiment 31 or 32, wherein the alteration in the phenotypic trait is increased in comparison to a control plant to which a Methylobacterium lacking a recombinant DNA construct had been applied.
34. A method of altering a phenotypic trait in a host plant comprising the step of applying the composition of embodiment 10 or 30, to a plant or a plant part.
35. The method of embodiment 34, wherein said plant part is a seed.
36. A method for inhibiting a plant pest in a host plant comprising the step of applying the Methylobacterium of any one of embodiments 1-11 or the engineered Methylobacterium of any one of embodiments 13-22 to a plant, a plant part, and/or to soil in which the plant will be grown or plant part deposited.
37. The method of embodiment 36, wherein said plant part is a seed.
38. The method of embodiment 36 or 37, wherein the inhibition of the plant pathogen is increased in comparison to a control plant to which a Methylobacterium lacking a recombinant DNA construct had been applied.
39. A method for inhibiting a plant pathogen in a host plant comprising the step of applying the composition of embodiment 10 or 30 to a plant, a plant part, and/or to soil in which the plant will be grown or plant part deposited.
40. The method of embodiment 39, wherein said plant part is a seed.
41. The method of embodiment 39 or 40, wherein the inhibition of the plant pathogen is increased in comparison to a control plant to which a composition containing Methylobacterium lacking a recombinant DNA construct had been applied.

### Embodiment List 4

1. A method of producing a transconjugant Methylobacterium isolate, comprising: incubating (i) a donor Methylobacterium isolate comprising a mobilizable plasmid containing a marker; and (ii) a recipient Methylobacterium isolate; wherein the mobilizable plasmid has an origin of replication functional in the recipient Methylobacterium isolate; wherein said mobilizable plasmid is transferred from said donor Methylobacterium isolate to said recipient Methylobacterium isolate; and screening cells of said recipient Methylobacterium isolate for the presence of the mobilizable plasmid marker to identify a transconjugant Methylobacterium isolate.
2. The method of embodiment 1, wherein said marker is a selectable marker.
3. The method of embodiment 2, wherein said selectable marker is a gene encoding resistance to an antibiotic.
4. The method of embodiment 1, wherein said marker is a genetic sequence marker.
5. The method of embodiment 1, wherein said marker is a screenable marker.
6. the method of embodiment 5, wherein said screenable marker encodes a fluorescent protein.
7. The method of any one of embodiments 1-6, wherein said mobilizable plasmid is a native Methylobacterium plasmid.
8. The method of any one of embodiments 1-7, wherein said method further comprises the use of a helper strain, wherein said helper strain encodes conjugation transfer functions.
9. The method of any one of embodiments 1-8, wherein said recipient Methylobacterium isolate contains a mutation in the carotenoid biosynthesis pathway.
10. The method of embodiment 9, wherein said mutation results in loss of function of crtI.
11. The method of any one of embodiments 1-9, wherein said origin of replication is an RK2 origin of replication.
12. A method of producing a population of transconjugant Methylobacterium isolates, comprising the steps of: (i) incubating a composition comprising a first donor Methylobacterium isolate comprising a mobilizable plasmid containing an origin of replication functional in Methylobacterium and a marker, and one or more recipient Methylobacterium isolates under conditions wherein said mobilizable plasmid is transferred from said donor Methylobacterium isolate to said recipient Methylobacterium isolate or isolates; and (ii) screening cells of said recipient Methylobacterium isolate or isolates for the presence of the mobilizable plasmid marker to identify one or more transconjugant Methylobacterium isolates.
13. The method of embodiment 12, wherein said marker is a selectable marker or screenable marker.
14. The method of embodiment 12 or 13, wherein said composition comprises a one or more additional donor Methylobacterium isolates comprising a mobilizable plasmid containing an origin of replication functional in Methylobacterium and a marker.
15. The method of embodiment 14, wherein the marker on the mobilizable plasmid in said first donor Methylobacterium isolate is the same marker as on the mobilizable plasmid in said one or more additional Methylobacterium isolates.
16. The method of embodiment 15, wherein the marker on the mobilizable plasmid in said first donor Methylobacterium isolate is a different marker than the marker on the mobilizable plasmid in said one or more additional donor Methylobacterium isolates.
17. The method of embodiment 14, wherein the mobilizable plasmids of said first and additional donor Methylobacterium isolates each comprise a different marker.
18. A method of producing a transformed Methylobacterium isolate, comprising: transforming a recipient Methylobacterium isolate with a plasmid having an origin of replication functional in the recipient Methylobacterium isolate and a marker; wherein said plasmid is transferred to said recipient Methylobacterium isolate; and screening cells of said recipient Methylobacterium isolate for the presence of the marker to identify a transformed Methylobacterium isolate.
19. The method of embodiment 18, wherein said marker is a genetic sequence marker.
20. The method of embodiment 18 or 19, wherein said plasmid is a native Methylobacterium plasmid.
21. The method of any one of embodiments 18-20, wherein transforming is selected from the group consisting of electroporation, heat shock, ultra-sound, and transduction.
22. A Methylobacterium comprising a recombinant DNA construct wherein a promoter is operably linked to a heterologous sequence encoding a nucleic acid that can trigger an RNAi response.
23 The Methylobacterium of embodiment 22, wherein said RNAi response inhibits expression of a target plant pest gene.
24. The Methylobacterium of embodiment 22 or 23, wherein said Methylobacterium further comprises a recombinant DNA construct wherein a promoter is operably linked to a heterologous sequence comprising a nucleic acid that encodes a pesticidal or herbicide tolerance protein.
25. The Methylobacterium of any one of embodiments 22-24, wherein said RNAi response inhibits expression of a target plant gene.
26. The Methylobacterium of any one of embodiments 22-24, wherein said promoter is an inducible promoter.
27. The Methylobacterium of embodiment 26, wherein said inducible promoter is a glyphosate inducible promoter.
28. The Methylobacterium of embodiment 27, wherein said glyphosate inducible promoter is selected from the group consisting of a trp, pheA, tyrA, tyrB, aroA, aroB, aroC, aroD, aroE, aroF, aroG, aroH, aroK, and an aroL promoter.
29. The Methylobacterium of any one of embodiments 22-28, wherein said Methylobacterium further comprises a recombinant DNA construct wherein an inducible promoter is operably linked to a heterologous sequence that provides for partial or complete lysis of said Methylobacterium upon exposure to an agent that induces the promoter.
30. The Methylobacterium of embodiment 29, wherein said inducible promoter that is operably linked to a heterologous sequence that provides for partial or complete lysis of said Methylobacterium is a glyphosate inducible promoter.
31. The Methylobacterium of embodiment 30, wherein said glyphosate inducible promoter that is operably linked to a heterologous sequence that provides for partial or complete lysis of said Methylobacterium is selected from the group consisting of an trp, pheA, tyrA, tyrB, aroA, aroB, aroC, aroD, aroE, aroF, aroG, aroH, aroK, and an aroL promoter.
32. The Methylobacterium of any one of embodiments 29-31, wherein said heterologous sequence that provides for partial or complete lysis of said Methylobacterium encodes an enzyme selected from the group consisting of lysozyme, a 26 kD peptidoglycan hydrolase, an N-acetylmuramidase, an N-acetylglucosaminidase, an N-acetylmuramyl-1-alanine amidases, and an endotransglycosidase.
33. A composition comprising the Methylobacterium of any one of embodiments 22-32 and at least one agriculturally acceptable excipient or adjuvant.
34. A transformed Methylobacterium strain that comprises a selected host Methylobacterium strain or variant thereof comprising:
i) a first recombinant DNA construct wherein a promoter is operably linked to at least one heterologous sequence encoding a nucleic acid that can trigger an RNAi response, and
ii) a second recombinant DNA construct wherein a promoter is operably linked to a heterologous sequence comprising a nucleic acid that encodes a pesticidal or herbicide tolerance protein.
35. The transformed Methylobacterium of embodiment 34, wherein said RNAi response inhibits expression of a target plant pest gene and wherein said pesticidal protein is active against a target plant pest comprising the target plant pest gene.
36. The transformed Methylobacterium of embodiment 34 or 35, wherein said target plant pest is an insect pest or a pest that causes a plant disease.
37. The transformed Methylobacterium of embodiment 36, wherein said insect pest is a Coleopteran, Lepidopteran, and/or Hemipteran species pest.
38. The transformed Methylobacterium of embodiment 36, wherein said pest that causes a plant disease is a fungus, bacteria, virus and/or nematode pest.
39. The transformed Methylobacterium of any one of embodiments 34-38, wherein said RNAi response inhibits expression of a gene in a first target plant pest and wherein said pesticidal protein is active against a second target plant pest.
40. The transformed Methylobacterium of embodiment 39, wherein said first and second target plant pests are insect pests.
41. The transformed Methylobacterium of embodiment 40, wherein said insect pests are Coleopteran, Lepidopteran. and/or Hemipteran species pests.
42. The transformed Methylobacterium of any one of embodiments 34-39, wherein said first and second target plant pests are pests that cause a plant disease.
43. The transformed Methylobacterium of any one of embodiments 34-39, wherein said pests that cause a plant disease are fungi, bacteria, virus and/or nematode pests.
44. The transformed Methylobacterium strain of any one of embodiments 34-43, wherein said selected host Methylobacterium strain or variant thereof exhibits or is selected for improved desiccation tolerance, improved agricultural chemistry tolerance, and/or improved colonization efficiency in comparison to a control Methylobacterium strain.
45. The transformed Methylobacterium strain of embodiment 44, wherein said selected host Methylobacterium strain or variant thereof is an effective colonizer of a plant shoot.
46. The transformed Methylobacterium strain of embodiment 45, wherein said plant is soy and said selected host Methylobacterium strain or variant thereof is NLS0064 or a variant thereof.
47. The transformed Methylobacterium strain of embodiment 44, wherein said selected host Methylobacterium strain or variant thereof is an effective colonizer of plant roots.
48. The transformed Methylobacterium strain of embodiment 47, wherein said plant is corn and said selected host Methylobacterium strain or variant thereof is NLS0042 or a variant thereof.
49. The transformed Methylobacterium strain of any one of embodiments 34-43, wherein said selected host Methylobacterium strain or variant thereof is a mutant strain lacking RNAse III activity.
50. The transformed Methylobacterium strain of embodiment 49, wherein said selected host Methylobacterium strain or variant thereof is NLS0476 or a variant thereof.
51. A composition comprising the transformed Methylobacterium of any one of embodiments 34-43, and at least one agriculturally acceptable excipient or adjuvant.
52. A method of altering a phenotypic trait in a host plant comprising the step of applying the Methylobacterium of any one of embodiments 22-32, the composition of embodiment 33, or the transformed Methylobacterium of any one of embodiments 34-43 to a plant or a plant part.
53. The method of embodiment 52, wherein said plant part is a seed.
54. The method of embodiment 52 or 53, wherein the alteration in the phenotypic trait is increased in comparison to a control plant to which a Methylobacterium lacking a recombinant DNA construct had been applied.
55. A method of altering a phenotypic trait in a host plant comprising the step of applying the composition of embodiment 33, to a plant or a plant part.
56. The method of embodiment 55, wherein said plant part is a seed.
57. A method of altering a phenotypic trait in a host plant comprising the step of applying the composition of embodiment 51, to a plant or a plant part.
58. The method of embodiment 57, wherein said plant part is a seed.
59. A method for inhibiting a plant pest in a host plant comprising the step of applying the Methylobacterium of any one of embodiments 22-32 or the transformed Methylobacterium of any one of embodiments 34-43 to a plant, a plant part, and/or to soil in which the plant will be grown or plant part deposited.
60. The method of embodiment 59, wherein said plant part is a seed.
61. The method of embodiment 59, wherein the inhibition of the plant pest is increased in comparison to a control plant to which a Methylobacterium lacking a recombinant DNA construct had been applied.
62. A method for inhibiting a plant pest or plant pathogen in a host plant comprising the step of applying the composition of embodiment 33 to a plant, a plant part, and/or to soil in which the plant will be grown or plant part deposited.
63. The method of embodiment 62, wherein said plant part is a seed.
64. The method of embodiment 62 or 63, wherein the inhibition of the plant pest is increased in comparison to a control plant to which a composition containing Methylobacterium lacking a recombinant DNA construct had been applied.
65. A method for inhibiting a plant pest in a host plant comprising the step of applying the composition of embodiment 51 to a plant, a plant part, and/or to soil in which the plant will be grown or plant part deposited.
66. The method of embodiment 65, wherein said plant part is a seed.
67. The method of embodiment 65, wherein the inhibition of the plant pest is increased in comparison to a control plant to which a composition containing Methylobacterium lacking a recombinant DNA construct had been applied.
68. A method of detecting the presence of (a)Methylobacterium strain NLS0042 or a variant thereof; or (b) NLS0064 a variant thereof in a sample comprising detecting the presence in the sample of a nucleic acid comprising or located within: (i) SEQ ID NO:14, 15, and/or 16; or (ii) SEQ ID NO: 17, 18, or 19, respectively.
69. The method of embodiment 68, wherein the detecting of the nucleic acid comprises a polymerase chain reaction, branched DNA, ligase chain reaction, transcription mediated amplification (TMA), nucleic acid sequence-based amplification (NASBA), nanopore-, mass spectroscopy, hybridization, or direct sequencing based method, or any combination thereof.
70. The method of embodiment 68, said detection comprises the steps of:
(i) contacting the sample or DNA obtained therefrom with a DNA primer pair, wherein said primer pair comprises forward and reverse primers for amplification of a DNA fragment comprising or located within SEQ ID NO:14, 15, 16, 17, 18, or 19, thereby generating a DNA fragment,
(ii) contacting said DNA fragment with a probe specific for the presence of said DNA fragment, and
(iii) comparing the results of said contacting with positive and negative controls to determine the presence of in said sample.
   71. The method of embodiment 68, wherein said sample is a plant material that was treated with one or more of Methylobacterium strains selected from NLS0042 or NLS0064.
   72. The method of embodiment 68, wherein said plant material is leaves, roots or seeds.
   73. The method of embodiment 68, wherein the plant material is a processed plant product from a plant treated with one or more Methylobacterium strains selected from NLS0042 or NLS0064.
   74. The method of embodiment 68, wherein said sample is a soil sample.
   75. A plant part which is at least partially coated with a composition comprising the Methylobacterium of any one of embodiments 22-32, the composition of embodiment 33, or the transformed Methylobacterium of any one of embodiments 34-43.
   76. The plant part of embodiment 75, wherein the plant part is a seed, leaf, root, stem, tuber, flower, or fruit.
   77. The plant part of embodiment 75, wherein the plant part is a corn, soybean. Brassica sp. (, alfalfa, rice, rye, wheat, barley, oats, sorghum, millet, sunflower, safflower, tobacco, potato, peanut, or cotton plant part.

The following examples are offered by way of illustration and not by way of limitation.

### EXAMPLES

### Example 1 Desiccation Tolerance Screen

Methylobacterium isolates were screened to identify strains that are tolerant to desiccation and/or chemicals commonly used in agriculture to provide strains useful for improving crop production. Greater than 1000 strains were screened for desiccation tolerance as defined by percent viability after drying for 7 hours under sterile air in a laminar flow hood. Desiccation tolerance was rated using a score of 0, 1, 2 or 3 with “3” being the most tolerant.

The desiccation tolerance screen was conducted as follows. Bacterial strains to be tested are grown for 3-5 days at either 25 or 30° C. in 96 well plates in AMS-GuPP (ammonium minimal salts—Glutamate Phytopeptone) media. For the “Dry” sample, 10 ul of each bacterial sample was spotted to Row A in a new 96 well plate. Four samples were analyzed per plate with 3 reps per sample. The plate was allowed to dry open in a laminar flow hood for 7 hrs. The dried bacteria were then titered as follows:

100 ul of media (AMS-GluPP) was added to Row A. The plates were allowed to sit for 20 minutes to ensure complete resuspension and pipetted up and down with fresh pipette tips. 90 ul of media was added to rows B-H. With new pipette tips, 10 ul was transferred from Row A to Row B; solution in wells was pipetted up and down and tips were disposed. Process was repeated for rows C-H until a full dilution set was made for all plates. 10 ul was discarded from Row H at the end of each dilution, and the dilution set ranged from 10⁻¹ to 10⁻⁸. The initial cultures, “Pre-dry” samples, were titered in new 96 well plates using the above plate based titer method about 5.5 hours after placing the “Dry” sample plates in the laminar flow hood so that the dry and pre-dry plates incubated an even amount of time.

Plates were sealed with Microseal ‘B’ plate tape and placed in a 30° C. shaker for 4-5 days depending on growth rate. All plates were visualized on an Epson scanner, ensuring that the PreDry and Dry corresponding plates were together when scanned for efficient analysis. Plates were analyzed using the Most Probable Number (MPN) Method and scores recorded for analysis. Percent viability of “Dry” versus “Pre-Dry” samples was determined for each sample and averaged for each of the 3 replicates. The % viability score was converted to a desiccation tolerance (DT) score between 0 and 3 by multiplying the percent viability by 0.03. Strains with a DT value of greater than or equal to 1.5 were identified as desiccation tolerant.

### Example 2 Ag Chemistry Tolerance Screen

The Methylobacterium strains screened for desiccation tolerance as described above were also screened for tolerance to the commonly used chemicals iLeVO® (fluopyram—Bayer CropScience), Axyl Shield (metalaxyl—Sharda USA), Headline® (pyraclostrobin—BASF) and Xtendimax® (dicamba—Monsanto Technology LLC) using a plate assay as follows. Agar plates containing AMS-GIuPP media plus one of the below listed chemicals were prepared with concentrations calculated to approximate the amount that each seed would be exposed to in the field at the middle recommended treatment rate. Concentrations of the chemicals in the plates were:

TABLE 5

| | | | Treatment | Chemical Rate in |
|---|---|---|---|---|
| Chemistry | Concentration | | used in Field | Field (Mid-rate) |
| ILeVO (Fluopyram) | 3.596 | mL/L | Seed Treatment | 1.0 fl oz/100 lbs |
| | | | | seed |
| Axyl Shield | 0.986 | mL/L | Seed Treatment | 1.58 fl oz/140,000 |
| (Metalaxyl) | | | | seeds |
| Xtendimax (Dicamba) | 160.8 | uL/L | Foliar Spray | 22 fl oz/Acre* |
| Headline | 65.8 | uL/L | Foliar Spray | 9 fl oz/Acre* |
| (Pyraclostrobin) | | | | |

*used 10× rate

Bacterial strains to be tested were grown for 3-5 days at either 25 or 30° C. in 96 well plates in AMS-GIuPP media. Using a p200 multichannel pipette set to 175 uL, cultures were pipetted up and down approximately 10 times to ensure uniform turbidity throughout. Plates were spotted carefully (to avoid puncturing agar) using a p20 multichannel pipette set to 3.2 uL and dispensed until the first stop only to prevent excess spray spots on the plates. Three replicate plates were spotted for each of the strains to be tested. Plates were allowed to fully dry and then inverted and incubated for 5-7 days at room temperature or 30° C. Following incubation, plates were scanned using an Epson scanner. Growth of Methylobacterium on plates was visually scored using a rating of 0-3, 0 representing no growth and 3 representing full growth. Control plates were used for comparison to the AgChem plates to ensure accuracy. Scores for each of the three reps were averaged. Strains with a score of greater than or equal to 1.66 were identified as tolerant for a given ag chemical. Only those strains with a score of >1.66 for each of the ag chemicals tested were considered tolerant to agricultural chemicals.

### Example 3 Soybean Colonization Screen

To determine an appropriate target concentration of Methylobacterium for use in a colonization efficiency screen, seven strains previously identified as either having the ability to colonize soybean significantly at 10⁶ CFU/seed (4 strains) or as demonstrating poor colonization of soybean seeds when inoculated at 10⁶ CFU/seed (3 strains), were evaluated. Soybean seeds were treated at both 10⁵ and 10⁶ CFU/seed with each of the strains. Three repetitions were planted with 6 seeds per treatment per repetition. Results demonstrate that inoculation at a target seed titer of 10⁵ CFU/seed is useful for identification of Methylobacterium strains with ability to colonize soybean shoots at a significantly higher density than control treatments and other strains previously shown to be poor colonizers of soybean shoots. Methylobacterium isolates that colonize the shoot surfaces of soy most densely when applied to seed at a dose of 10⁵ CFU/seed (a reduction from the 10⁶ CFU/seed levels used previously in field trials) are identified as follows.

Methylobacterium strains that scored as tolerant in both desiccation tolerance and ag chemistry tolerance screens as described above in Examples 1 and 2 were tested for their ability to efficiently colonize soybean shoots. Soybean seeds were treated with Methylobacterium strains at a target seed titer of 10¹ CFU/seed. Flo-Rite 1706 polymer was used to stick microbe to seed. Each experiment included 10 Methylobacterium strains, plus an untreated control treatment (UTC) and a strain that was shown in the past to have limited ability to colonize soybean phyllosphere (NLSO400, the “negative control”). In each experiment, two seeds per pot were planted in unamended field soil, with 20 pots per treatment level in a randomized complete block design, resulting in a total of 240 pots per experimental run. Plants were grown for 2 weeks in a greenhouse at 25° C. with regular watering and no fertilizer. At harvest, the two plants from each pot were cut at ˜1 cm above the soil surface and placed into a 50 mL conical tube with 15 mL of 0.9% saline solution. Ten pots per treatment were sampled. Each tube was weighed before and after plant sampling to quantify plant fresh weight. Samples were vortexed for 15 minutes, then placed into an ultrasonic bath for 10 minutes. Samples were then plated onto AMS-MC using an easy Spiral automatic diluter and plater (Interscience, Inc.) at 5 dilutions, and plates were incubated for 8 days at 30° C. Plates were counted using a Scan 4000 automatic colony counter (Interscience, Inc.) to quantify the number of pink colonies. Results were recorded as the number of CFUs per mg of plant fresh weight.

Colonization density data (CFU/mg) were used to compare the strains in each run to the untreated control. A Mann-Whitney U-test was used to generate p-values comparing each treatment to the untreated control. The threshold for statistical significance used in this screen was p<0.05. Strains with significantly greater CFU/mg than the UTC were classified as “hits” based on which treatments were significantly higher than the negative control at p<0.05 using a Mann-Whitney U-test.

In some runs, the untreated control showed an unusually high value. The NLSO400 negative control was used as the standard for statistical comparison in any run in which 2 treatments or more showed mean CFU/mg lower than the UTC. Typically, hits colonized the shoot surfaces of soy at a rate that was 0.7-1.3 logs more CFUs per mg of plant fresh weight than the UTC or poorly-colonizing strains. Strains were considered poor colonizers and called “non-hits” if they displayed quantitatively lower colonization than the negative or untreated control.

### Example 4 Method of Electroporation

Electro-competent cells of Methylobacterium isolates were prepared as described by Toyama et al. (1998) with slight modifications. Cells were grown in AMS-GuPP medium until the culture reached an OD 600 of 0.6-0.8. Cells are harvested by centrifugation (1800 g, 10 min, 4° C.), washed once in sterile H₂O and twice with ice-cold sterile 10% (v/v) glycerol solution. The cell suspension was concentrated 100-fold in 10% glycerol and kept at −80° C. Electro-competent cells (100 μl) were mixed with DNA solution (1p) and transferred into a cuvette chilled on ice. Electroporation was carried out using a GenePulser (BioRad) with the following parameters: 2 kV, 200 Ω, 25 μF for 1-mm gap cuvettes. Electro-shocked cells were then shaken at 30° C. for 4 hours and spread on AMS-GuPP plates with appropriate antibiotics.

### Example 5 Methods of Conjugation

Donor and recipient Methylobacterium isolates and optionally E. coli helper strain were co-incubated overnight on solid media at 30° C. using an appropriate ratio of the donor isolate, recipient isolate and helper strain, if required. Following the incubation, the conjugational bacterial mixture is plated on AMS-MC media plates with and without appropriate antibiotics.

### Construction of a Mobilizable Plasmid

The 19 bp TetR operator sequence was removed from pLC291 (Chubiz et al. (2013), and a synthetic mCherry coding sequence cloned downstream of the P_{R} promoter to provide for constitutive expression of the fluorescent marker protein. pQZ1024 contains ColE1 and RK2 origins of replication and an origin of transfer (oriT) recognizable by traJ. The plasmid encodes the traJ′ mutant that allows for efficient replication and/or transfer in Methylobacterium (Marx and Lindstrom (2001)) and contains a selectable marker for kanamycin resistance.

### Construction of Donor Methylobacterium Isolates

pQZ1024 was electroporated into NLS0064 (see Table 2) and to generate Methylobacterium donor isolates NLS89_mCherry and NLS64_mCherry (see Table 2). Electro-competent cells of Methylobacterium isolates were prepared as described above and mixed with pQZ1024 DNA. The cells were shaken at 30° C. for 4 hours and spread on an AMS-GuPP plate containing 50 mg/l kanamycin.

### Construction of Recipient Methlyobacterium Isolate

A colorless mutant Methylobacterium isolate was constructed by allelic exchange as follows. The NLS0020 CrtI gene (SEQ ID NO:42) was PCR amplified from NLS0020 (see Table 2) genomic DNA and mutated through PCR-based site-directed mutagenesis. The CrtI mutant gene has a 25 nt deletion of nucleotides 879-903 in the open reading frame of SEQ ID NO:42 and is designated CrtI_{NLS0020}Δ25 nt. CrtI_{NLS0020}Δ25 nt was cloned into pCM433 (Marx (2008)) through BglII and XhoI restriction sites, and designated pQZ1005. E. coli harboring pQZ1005 was conjugated into NLS0020 using the helper plasmid pRK2013. NLS0020 harboring pQZ1005 was counter selected on 5% sucrose agar plate for the homologous recombination between WT CrtI gene and CrtI_{NLS002}Δ25 nt, and loss of the transformed plasmid. The vector-free mutant no longer produces Methylobacterium intrinsic pink color and appears whitish. The mutant isolate is designated NLS0020_CrtI_{NLS0020}Δ25 nt (see Table 2) and assigned NLS isolate number mQZ3002.

Methylobacterium Conjugation and Screening with Donor Selectable Marker and Recipient Phenotype

Tri-parental conjugations were conducted using Methylobacterium isolate NLS0089_mCherry or NLS0064_mCherry as the donor isolate, Methylobacterium crtI mutant isolate mQZ3002 as the recipient isolate and an E. coli helper strain containing the conjugation plasmid pRK2013 which lacks a functional RK2 origin of replication and can only be maintained in E. coli.

The Methylobacterium isolates and E. coli helper strain were co-incubated for one day on AMS-GIuPP plates at 30° C. using a 1:1:1 ratio of the donor isolate, recipient isolate and helper strain. Following the incubation, the conjugational bacterial mixture was plated on AMS-MC media plates with and without 50 mg/l kanamycin.

Transconjugants were identified as resistant to kanamycin, lacking the pink color typical of Methylobacterium (some color glow from mCherry marker was visible even under white (visible) light), fluorescence due to mcherry expression, and morphology. Putative transconjugants were confirmed by colony PCR using NLS0020 strain specific and mcherry specific primers.

The frequency of conjugation was determined by dividing the number of successful transconjugants (growing on kan plate) by the number of donor colonies (growing on non-kan plate). The conjugation rate for transfer from donor isolate NLS0089 to recipient isolate NLS0020CniA25 nt was approximately 1:700. The conjugation rate for transfer from donor isolate NLS0064 to recipient isolate NLS0020CI5 nt was approximately 1:1300.

TABLE 6
Exemplary Methylobacterium Isolates

| Identifier | Species | Origin |
|---|---|---|
| NLS0020^{CrtIΔ25nt} | M. radiotolerans | A colorless mutant |
| | | Methylobacterium isolate |
| | | derived from NLS0020 |
| NLS0064_mCherry | M. gregans | NLS0064 conjugated with |
| | | pQZ1024 mcherry marker |
| | | plasmid |
| NLS0089_mCherry | M. populi | NLS0089 conjugated with |
| | | pQZ1024 mcherry marker |
| | | plasmid |

### Example 6 Conjugation and Screen Using Donor Genetic Marker

Conjugation is conducted using Methylobacterium isolate NLS0089 as the donor, Methylobacterium isolate NLS0064 as the recipient at donor recipient ratios of 1:5, 1:10, or 1:20, with and without E. coli containing the conjugation helper plasmid pRK2013. Post-conjugation recipient Methylobacterium isolate NLS0064 colonies are identified from donor Methylobacterium isolate NLS0089 colonies on the basis of their larger colony size and lighter pink color. Identified colonies are screened by qPCR using NLS0089-specific primers.

### Example 7 Transformation of Recipient Methylobacterium with Donor Isolate Plasmid

Plasmids were isolated from NLS0042 having approximate sizes of 34 Kb, 31 Kb, and 11 Kb. The identity of the plasmids was confirmed by restriction digest and qPCR. Intactness of the plasmids was confirmed using a plasmid safe DNase treatment. DIG-labeled probes specific for sequences in the NLS0042 plasmids were prepared as found in DIG Application Manual for Filter Hybridization by Roche, and Viterbo et al. (2018).

The plasmids were electroporated into recipient Methylobacterium NLS0089 as described in Example 4. NLS0089 colonies that received a plasmid from NLS0042 were identified by two different methods:

1) By colony hybridization techniques using DIG-labeled probes that are specific to plasmids in NLS0042.
2) By dilution to extinction plating techniques to plate one to twenty electroporation colonies in each well of 96 well plates, followed by colony qPCR using primers specific to plasmids in NLS0042. Positive wells will be restreaked and the individual colonies checked by colony qPCR to identify the exact NLS0089 colonies containing the transformed NLS0042 plasmid.

### Example 8 Transformed Methylobacterium for Insect Resistance

Lepidopteran, Coleopteran and Dipteran insect pests cause significant losses of yield in a number of crops. Some of these insect pests feed on foliar and root tissue efficiently colonized by the Methylobacterium spp. A Methylobacterium strain capable of colonizing the root maize tissue will be transformed with a dsRNA-expressing construct directed against an essential gene vacuolar ATPase subunit A for the growth and development of corn rootworm (Baum J et al. (2007). The bacteria expressing the dsRNA will be applied to the corn seed and the seed planted in 12-inch pots in the greenhouse. Roots of the corn plants will be tested for efficient colonization by the transformed strain and tested for the expression of dsRNA specific for the gene. Plants expressing dsRNA will be inoculated with the larvae of corn root worm. As controls, seed will be treated with the Methylobacterium strain containing empty plasmid DNA. The inoculated plants will then be tested for resistance to root damage by corn root worm. It is likely that dsRNA will be taken up by the insect larvae feeding on the roots and trigger RNAi mediated silencing of the essential gene, thus controlling this insect pest.

### Example 9 Transformed Methylobacterium for Nematode Resistance

Plant parasitic root knot (Meloidogyne spp.) and cyst (Heterodera spp.) nematodes cause significant losses of yield in all major crops such as legumes, vegetables and cereals. These nematodes colonize the roots and cause extensive damage by feeding on them. A Methylobacterium strain colonizing the tomato root tissue will be transformed with a dsRNA-expressing construct directed against a gene essential for the normal development of M. incognita root-knot nematodes and the establishment of a nematode population. Examples of such essential nematode genes include, but are not limited to, cysteine proteinase gene or dual oxidase gene (Karakas M (2008). The Methylobacterium expressing the dsRNA will be applied to tomato seed and the seed planted in 12-inch pots in the greenhouse. Roots of tomato plants will be tested for efficient colonization by the transformed strain and tested for the expression of dsRNA specific for either gene. Tomato roots colonized with Methylobacterium expressing dsRNA will be inoculated with the larvae of M. incognita. As controls, seed will be treated with the Methylobacterium strain containing empty plasmid DNA that does not produce the dsRNA. The inoculated plants will then be tested for resistance to root damage by M. incognita and/or reduced reproduction of M. incognita. The dsRNA will be taken up by the nematode larvae feeding on the roots, trigger RNAi mediated silencing of the essential gene and provide resistance to this nematode pest.

Similar experiments will be conducted to demonstrate resistance to soybean cyst nematode (H. glycines). In this case, RNAi will be directed against a gene of the cyst nematode. Examples of cyst nematode genes that can be targeted to provide nematode control include, but are not limited to, cysteine proteinase, C-type lectin or the β-1,4-glucanase gene (Karakas M (2008).

### Example 10 Transformed Methylobacterium for Fungal Resistance

Fungal pathogens are responsible for devastating losses of yield in several crops. Thus, biotrophic, hemibiotrophic and necrotrophic fungal pathogens cause on average 10-15% losses of yield every year globally. For example, biotrophic fungal pathogens of the genus Puccinia cause infection of the leaf or stem tissue of wheat and significantly reduce seed yield at the end of the growing season. A Methylobacterium strain capable of colonizing the wheat leaf tissue will be transformed with a dsRNA-expressing recombinant DNA construct directed against the PsCNA1 and PsCNB1 genes encoding the subunits of calcineurnin P. striiformis. These genes have been shown to be essential for stripe rust morphogenetic differentiation particularly during haustoria formation and production of urediospores (Zhang H et al. (2012). Bacteria expressing the dsRNA directed against these genes will be applied as a foliar spray to the leaves of wheat plants grown in 3-inch pots in a growth chamber. Leaves of sprayed plants will be tested for efficient colonization by the transformed strain, tested for the expression of dsRNA specific for the gene and subsequently challenged with the conidia of the strip rust fungus. As controls, leaves of wheat plants will be treated with the Methylobacterium strain containing empty plasmid DNA. The inoculated plants will then be tested for resistance to stripe rust using the well-established disease rating protocol.

### Example 11 Transformed Methylobacterium for Virus Resistance

Methylobacterium delivered RNAi technology can also be potentially applied for engineering resistance to plant RNA and DNA viruses. The Methylobacterium can be transformed to express dsRNA directed against the replicase or the coat protein gene of either an RNA virus or a DNA virus. These viral targets are susceptible to RNAi mediated inhibition (Godge M R et al. (2008). As an example, potato leaf colonizing Methylobacterium strain can be transformed to deliver dsRNA directed against the replicase gene of potato leaf roll virus (PLRV). This PLRV target has also been validated (Rovere C V et al. (2001). Foliar application of this Methylobacterium strain on Russet Burbank potato would likely induce RNAi directed against this essential gene for PLRV replication and spread in potato.

### Example 12 Expression of Bt Toxins

Toxin genes for cloning into Methylobacterium strains are codon optimized to match codon usage frequency of Methylobacterium. To provide for constitutive expression, the phage PR promoter is modified during the cloning process to delete the TetR. Sequence (SEQ ID NO:43) of the modified PR promoter is shown below.

SEQ ID NO: 43
TGCATCCCAACAACTTATACCATGGCCTACAAAAAGGCAAACAATGGTAC
TTGACGACTCATCACAACAATTGTAGTTGTAGATTGTAAT

The active portion of the Cry1a endotoxin (amino acids 29-605) was codon optimized, synthesized and cloned in vector pLC291 (Chubiz et al. (2013)) for constitutive expression under control of the modified phage PR promoter. The sequence of the synthesized codon optimized Cry1Aa gene is provided as SEQ ID NO:4.

The entire Cry1Ac gene is codon optimized, synthesized and cloned in vector pLC291 for constitutive expression under control of the modified phage PR promoter. The sequence of the codon optimized CryIAc1 gene is provided as SEQ ID NO:46.

NLS0064 was conjugated with E. coli carrying pLC291_Cry1a and E. coli carrying the helper plasmid as follows. Single colonies of donor, helper and recipient strains were spread on appropriate growth medium plates until a thin layer of bacterial lawn was established. An equal volume of each strain is scooped, suspended in 1 ml 0.9% saline buffer, and centrifuged at 10000 rpm for 1 min. Pellet chunks are spread onto an AMS-GluPP plate for overnight incubation. Tri-parental conjugants were selected on selective medium plate containing appropriate antibiotics for successful conjugation and transformed NLS0064 conjugants carrying pLC291_Cry1a identified by colony PCR followed by Sanger sequencing.

Transformed NLS0064 carrying pLC291_Cry1a was also prepared by electroporation as follows. Electro-competent cells of M-trophs were prepared by the method of Toyama et al. (1998) with slight modifications. Cells were grown in AMS-GuPP medium until the culture reached an OD 600 of 0.6-0.8. Cells were harvested by centrifugation (1800 g, 10 min, 4° C.), washed once in sterile H2O and twice with ice-cold sterile 10% (v/v) glycerol solution. The cell suspension was concentrated 100-fold in 10% glycerol and kept at −80° C. Electro-competent cells (100 μl) were mixed with DNA solution (1p) and transferred into a cuvette chilled on ice. Electroporation was carried out using a GenePulser (BioRad) with the following parameters: 2 kV, 200 Ω, 25 ρF for 1-mm gap cuvettes. Electro-shocked cells were then shaken at 30° C. overnight and were spread on an AMS-GuPP plate with appropriate antibiotics.

Transformed strains are tested to determine efficacy against insect pests and applied to plant seeds to deliver the insecticidal proteins to the plant and/or plant growing environment.

### Example 13 Detection of Methylobacterium Strains

Assays are disclosed for detection of specific Methylobacterium strains and closely related derivatives.

A qPCR Locked Nucleic Acid (LNA) based assay for NLS109 was developed as follows. NLS109 genomic DNA sequence was compared by BLAST analysis of approximately 300 bp fragments using a sliding window of from 1-25 nucleotides to whole genome sequences of over 1000 public and proprietary Methylobacterium isolates. Genomic DNA fragments were identified that had weak BLAST alignments, indicative of approximately 60-95% identity over the entire fragment, to corresponding fragments from NLSO109. Target fragments from the NLSO109 genome corresponding to the identified weak alignments regions that were selected for assay development are provided as SEQ ID NOS:11-13.

TABLE 7
Target Fragment Sequences of NLS0109

| | SEQ | |
|---|---|---|
| | ID | |
| Fragment | NO | Sequence |
| ref1_135566 | 11 | ACGGTCACCCCACGGACTGGGCGAGTACCTCACCGGTGT |
| | | TCTATCATAACGCCGAGTTAGTTTTCGACCGTCCCTTATG |
| | | CGATGTACCACCGGTGTCGGCAGCCGATTTCGTCCCACC |
| | | GGGAGCTGGCGTTCCGGTTCAGACCACCATCATCGGTCA |
| | | CGATGTCTGGATTGGACACGGGGCCTTCATCTCCCCCGG |
| | | CGTGACTATAGGAAACGGCGCGATCGTCGGGGCCCAGG |
| | | CGGTCGTCACAAGAGATGTCCCACCCTATGCGGTAGTTG |
| | | CTGGCGTCCCCGCGACCGTACGACGAT |
| ref1_135772 | 12 | CCAATAAAAGCGTTGGCCGCCTGGGCAACCCGATCCGA |
| | | GCCTAAGACTCAAAGCGCAAGCGAACACTTGGTAGAGA |
| | | CAGCCCGCCGACTACGGCGTTCCAGCACTCTCCGGCTTT |
| | | GATCGGATAGGCATTGGTCAAGGTGCCGGTGGTGATGAC |
| | | CTCGCCCGCCGCAAGCGGCGAATTACTCGGATCAGCGGC |
| | | CAGCACCTCGACCAAGTGTCGGAGCGCGACCAAAGGGC |
| | | CACGTTCGAGGACGTTTGAGGCGCGACCAGTCTCGATAG |
| | | TCTCATCGTCGCGGCGAAGCTGCACCTCGA |
| ref1_169470 | 13 | CGATGGCACCGACCTGCCATGCCTCTGCCGTCCGCGCCA |
| | | GAATGGTAAAGAGGACGAAGGGGGTAAGGATCGTCGCT |
| | | GCAGTGTTGAGCAGCGACCAGAGAAGGGGGCCGAACAT |
| | | CGGCATCAAACCTCGATTGCCACTCGGACGCGAAGCGCG |
| | | TCTTGAAGGAGGGATGGAAGCGAAACGGCCGCAGAGTA |
| | | ACCGCCGACGAAAGATTGCACCCCTCATCGAGCAGGATC |
| | | GGAGGTGAAGGCAAGCGTGGGTTATTGGTAAGTGCAAA |
| | | AAATATAATGGTAGCGTCAGATCTAGCGTTC |

Regions in SEQ ID NOS: 11-13 where corresponding regions in other Methylobacterium strains were identified as having one or more nucleotide mismatches from the NLS109 sequence were selected, and qPCR primers designed using Primer3 software (Untergasser et al. (2012), Koressaar et al. (2007) to flank the mismatch regions, have a melting temperature (Tm) in the range of 53-58 degrees, and to generate a PCR DNA fragment of approximately 100 bp. The probe sequence was designed with a 5′ FAM reporter dye, a 3′ Iowa Black FQ quencher, and contains one to six LNA bases (Integrated DNA Technologies, Coralville, Iowa). At least 1 of the LNA bases is in the position of a mismatch, while the other LNA bases are used to raise the Tm. The Tm of the probe sequence is targeted to be 10 degrees above the Tm of the primers.

Primer and probe sequences for detection of specific detection of NLS0109 are provided as SEQ ID NOS: 47-55 in Table 8. Each of the probes contains a 5′ FAM reporter dye and a 3′ Iowa Black FQ quencher.

TABLE 8
Primer and Probe Sequences for Specific Detection of NLS0109

| | SEQ | |
|---|---|---|
| Primer/Probe | ID NO | Sequence* |
| NLS0109_ref1_135566_forward | 47 | CCTCACCGGTGTTCTATCATAAC |
| NLS0109_ref1_135566_reverse | 48 | CCGATGATGGTGGTCTGAAC |
| NLS0109_ref1_135566_probe | 49 | CGTCCCTTATGCGATGTACCA |
| NLS0109_ref1_135772_forward | 50 | GATCCGAGCCTAAGACTCAAAG |
| NLS0109_ref1_135772_reverse | 51 | GACCAATGCCTATCCGATCAA |
| NLS0109_ref1_135772_probe | 52 | AACACTTGGTAGAGACAGCC |
| NLS0109_ref1_169470_forward | 53 | AAGGAGGGATGGAAGCGAAAC |
| NLS0109_ref1_169470_reverse | 54 | ATAACCCACGCTTGCCTTC |
| NLS0109_ref1_169470_probe | 55 | CGCAGAGTAACCGCCGACGAA |

*Bold and underlined letters represent the position of an LNA base

Use of Primer/Probe Sets on Isolated DNA to Detect NLS0109 and Distinguish from Related Methlyobacterium Isolates

A qPCR reaction is conducted in 20 ul and contains 10 ul of 2×KiCqStart™ Probe qPCR ReadyMix™, Low ROX™ from Sigma (Cat #KCQS05-1250RXN), 1 ul of 20× primer-probe mix (final concentration of primers is 0.5 uM each and final concentration of probe is 0.25 uM), and 9 ul of DNA template/water. Approximately 30-40 ng of DNA template is used per reaction. The reaction is conducted in a Stratagene Mx3005P qPCR machine with the following program: 95° C. for 3 min, then 40 cycles of 95° C. for 15 sec and 60° C. for 1 min. The MxPro software on the machine calculates a threshold and Ct value for each sample. Each sample was run in triplicate on the same qPCR plate. A positive result is indicated where the delta Ct between positive and negative controls is at least 5.

Use of the three primer/probe sets to distinguish NLS0109 from closely related isolates by analysis of isolated DNA is shown in Table 9 below. The similarity score shown for the related isolates takes into account both the average nucleotide identity and the alignment fraction between the isolates and NLSO109. One of the tested strains, NLS0730, was used as an additional positive control. NLS0730 is a clonal isolate of NLS109 which was obtained from a culture of NLS0109, which was confirmed by full genome sequencing as identical to NLSO109, and which scored positive in all three reactions. The similarity score of greater than 1.000 for this strain is likely the result of a slightly different assembly of the genome for this isolate compared to NLSO109. The delta Ct of approximately 15 or more between the NLSO109 and NLS0730 isolates and the water only control is consistent with the sequence confirmation of the identity of these isolates. Analysis of other isolates that are less closely related to NLS0109 results in delta Ct values similar to those for the water only control.

TABLE 9

| | Similarity | | | |
|---|---|---|---|---|
| | score to | Average Ct Value | | |
| NLS# | NLS0109 | Ref1_135566 | Ref1_135772 | Ref1_169470 |
| NLS0730 | 1.005 | 21.08 | 21.31 | 20.35 |
| NLS0109 | 1 | 21.97 | 22.62 | 22.08 |
| NLS0731 | 0.181 | No Ct | 37.85 | >37.91 |
| NLS0644 | 0.87 | >36.8 | >38.31 | No Ct |
| NLS0700 | 0.88 | >38.36 | >38.36 | >38.44 |
| NLS0710 | 0.894 | No Ct | >37.47 | >38.13 |
| NLS0834 | 0.852 | 37.81 | No Ct | 37.97 |
| NLS0939 | 0.862 | 37.94 | 38.37 | >38.35 |
| NLS0947 | 0.807 | 38.44 | No Ct | No Ct |
| NLS1015 | 0.894 | 38.77 | No Ct | >37.91 |
| NLS1217 | 0.872 | 37.64 | 37.20 | 37.96 |
| H2O | | >38.14 | >35.92 | >37.12 |
| only | | | | |

### Use of Primer/Probes for Detection of NLS109 on Treated Plant Materials.

Detection of NLSO109 on Seed Washes from Treated Soybean Seeds.

NLSO109 can be detected and distinguished from other Methylobacterium isolates on treated soybean seeds as follows. Soybean seeds were treated with Methylobacterium isolates from 10× frozen glycerol stock to obtain a final concentration of 10 CFU/seed. Becker Underwood Flo Rite 1706 polymer is used to improve adhesion. An uninoculated control containing polymer and water is used. DNA is isolated from the seeds as follows. Approximately 25 ml of treated seeds are submerged for 5 minutes in 20 ml 0.9% sterile saline. Tubes are vortexed for 15 minutes, then the seed wash is removed to a new tube. An additional 10 ml 0.9% sterile saline is added to the same seeds, vortexed briefly, and combined with the previous seed wash. The seed wash liquid is centrifuged. The loose pellet is saved and transferred to smaller tubes, while the supernatant is discarded. The sample is centrifuged again, and the final sample obtained as an approximately 100 ul loose pellet. The 100 ul pellet is used as the input for DNA extraction using MOBio UltraClean Microbial DNA Extraction kit Cat #12224-250. As shown in Table 10, NLS0109 and NLS0730, are detected in seed washes from treated soybean seeds using all 3 primer probe sets, as demonstrated by delta Ct of greater than 10 as compared to Ct values of negative controls.

TABLE 10

| | Similarity | | | |
|---|---|---|---|---|
| | score to | Average Ct Value | | |
| Treatment | NLS0109 | Ref1_135566 | Ref1_135772 | Ref1_169470 |
| NLS0109 | 1 | 18.07 | 17.49 | 17.95 |
| control | N/A | 34.80 | 33.72 | 33.59 |
| (polymer | | | | |
| only) | | | | |
| NLS0730 | 1.005 | 17.76 | 17.03 | 17.54 |
| NLS0731 | 0.181 | 33.67 | 32.70 | 32.43 |

Detection of NLSO109 on Leaves from Plants Grown from Treated Soybean Seeds.

Soybean seeds were treated with Methylobacterium isolates NLSO109, NS0730, and NLS0731 from 10× frozen glycerol stock to obtain a final concentration of 10⁶ CFU/seed. Becker Underwood Flo Rite 1706 polymer is used to improve adhesion. An uninoculated control contained polymer and water. Seeds were planted in field soil mix, placed in a growth chamber for approximately two weeks, and watered with unfertilized RO water every 1-2 days to keep soil moist. After 2 weeks of growth, true leaves from about 9 plants were harvested into sterile tubes. Each treatment had at least 2 reps in each experiment, and each experiment was grown at least 3 times.

DNA from bacteria on the harvested leaves is isolated as follows. Leaves are submerged for 5 minutes in buffer containing 20 mM Tris, 10 mM EDTA, and 0.024% Triton X-100. Tubes are vortexed for 10 minutes, and then sonicated in two 5 minute treatments (10 minutes total). Leaf tissue is removed, and the remaining liquid centrifuged. The loose pellet is saved and transferred to smaller tubes, while the supernatant is discarded. The sample is centrifuged again, and the final sample obtained as an approximately 100 ul loose pellet. The 100 ul pellet is used as the input for DNA extraction using MOBio UltraClean Microbial DNA Extraction kit Cat #12224-250. The average yield of DNA is 50-60 ng/ul in 30 ul. As shown in Table 11, NLS0109 and NLS0730, are detected on leaves harvested from plants grown from soybean seeds treated with the Methylobacterium strains using all 3 primer probe sets, as demonstrated by delta Ct values of around 5.

TABLE 11
Average of 3 experiments each with 3 biological replicates

| | Similarity | | | |
|---|---|---|---|---|
| | score to | Average Ct Value | | |
| Treatment | NLS0109 | Ref1_135566 | Ref1_135772 | Ref1_169470 |
| NLS0109 | 1.000 | 35.00 | 34.67 | 34.00 |
| control | N/A | 39.67 | 39.67 | 39.33 |
| (polymer | | | | |
| only) | | | | |
| NLS0730 | 1.005 | 35.00 | 35.00 | 34.00 |
| NLS0731 | 0.181 | 40.00 | 39.67 | 40.00 |

For detection of NLS0109 foliar spray treatment on com: Untreated corn seeds were planted in field soil in the growth chamber and watered with non-fertilized R.O. water. After plants germinated and grew for approximately 3 weeks, they were transferred to the greenhouse. At V5 stage, plants were divided into 3 groups for treatment: foliar spray of NLSO109, mock foliar spray, and untreated. Plants receiving the foliar spray of NLS109 were treated with 10× glycerol stock at the rate of 71.4 ul per plant using Solo sprayers, his converts to the rate of 10 L/acre in the field. Mock treated plants were sprayed with 71.4 ul water/plant. Untreated plants received no foliar spray treatment. Leaves were harvested two weeks after foliar spray treatment into sterile tubes and DNA from bacteria on the harvested leaves is isolated as described above. Each experiment was grown at least 2 times. As shown in Table 12, NLSO109 is detected on leaves harvested from corn plants treated by a foliar spray application of the Methylobacterium strains using all 3 primer probe sets, as demonstrated by delta Ct values of approximately 10 between the sample and the negative controls.

TABLE 12

| | Average Ct Value | | |
|---|---|---|---|
| Treatment | Ref1_135566 | Ref1_135772 | Ref1_169470 |
| Control (no | 32.43 | 32.10 | 31.55 |
| application) | | | |
| Control (mock | 35.54 | 35.34 | 34.80 |
| application) | | | |
| NLS0109 | 23.36 | 22.88 | 22.66 |
| (10L/acre | | | |
| equivalent) | | | |

The above results demonstrate the use of genome specific primers and probes to detect Methylobacterium strain NLSO109 on various plant tissues following treatment with the strains and provide methods to distinguish NLSO109 from closely related isolates. Similar methods are developed for additional Methylobacterium strains, NLS0042 and NLS0064 using target sequence fragments and primer/probe pairs as shown in the Tables below.

TABLE 13
Target Fragment Sequences of NLS0042

| | SEQ | |
|---|---|---|
| | ID | |
| Fragment | NO | Sequence |
| ref1_86157 | 14 | AGCCCACAAGCCTGATGCACTTAACTACATCCTCTAATGTCGCGCC |
| | | AATTTGCTTGGCGGCAGGGGATGTTGTATCGTCATAGGCTTGTCTA |
| | | ACCGGAACTTGTTTGCCAATCTCTTTGGCGATCGCAACCGCCATCT |
| | | CGTGTTCGTCAACCATGTGCGCGTTCCTCTAATTGCACTCATGGTG |
| | | CCACGTGCACCTCCGATCGTCTCGTGTCTAGAATGAAGGTGGGAAC |
| | | AACCTTACACAGGCTTTCGCGACGCGCGAATTTCTGGTTTCTCCGC |
| | | CTCGGATGTGGGTTTGAGCGCTTC |
| ref1_142469 | 15 | CTTTTCATTTGTCATGATCTCGACCAAGGTATTCACGGCAA |
| | | GCTCGGTCTGTTGCTTAGCAAGTGCCTGAACTTCGCGAACG |
| | | ATCGGCTCTCGACCCTTCGGGTTCGAGACCTGTCCCTTTTG |
| | | AAAACCACGTGCCCTACACTTTTCGGGATCAAGGTGCGGGT |
| | | TGGCTTTGGTCAAAATTCTCTGGCGTCCCATTACACGCCCT |
| | | CCGCATCATCGTTCCCGCGAACGATCTGACCCCCGACTTCC |
| | | GCGAGGAAGCGTGTGGCGTGATCCTCGAAGCGGAATGCCA |
| | | CCTCGAACTGTTCC |
| ref1_142321 | 16 | CAGCAGCAAGCAGATCGTTGAAAACCGCTTGAACCGCATC |
| | | TTGATCGGGACCGGAACCAATCAGGTCATCTAGGTAAACC |
| | | GAGACGTAAACTCGTTTGCGCTCGGCATCTTTCAGAACGTC |
| | | CGTGATGCCAGACCGCATTAGTACCATCGTCGCCAAGGCG |
| | | GGCGACTGAACGAAGCCGATCGGCAGAGAGTAACGGGGA |
| | | CCGCCCCTAATCGGGTTGCGAACGCAAGACCACTTAGCAA |
| | | AGGTTCGAGCACGGCCGAACTTCGCATGGTGGAGAGCCGC |
| | | GGCAACACGGTTCCGTGATA |

TABLE 14
Primer and Probe Sequences for Specific Detection of NLS0042

| | SEQ | |
|---|---|---|
| | ID | |
| Primer/Probe | NO | Sequence* |
| NLS0042_ref1_86157_reverse | 56 | AAGCCTGTGTAAGGTTGTTCCC |
| NLS0042_ref1_86157_forward | 57 | CCATGTGCGCGTTCCTCTAAT |
| NLS0042_ref1_86157_probe | 58 | ACCTCCGATCGTCTCGTGTCT |
| NLS0042_ref1_142469_reverse | 59 | GTAATGGGACGCCAGAGAAT |
| NLS0042_ref1_142469_forward | 60 | TGCTTAGCAAGTGCCTGAA |
| NLS0042_ref1_142469_probe | 61 | AAGCCAACCCGCACCTTGAT |
| NLS0042_ref1_142321_reverse | 62 | CCGTGCTCGAACCTTTGCTA |
| NLS0042_ref1_142321_forward | 63 | CAGACCGCATTAGTACCATCGTC |
| NLS0042_ref1_142321_probe | 64 | CGGGTTGCGAACGCAAGAC |

*Bold and underlined letters represent the position of an LNA base

TABLE 15
Target Fragment Sequences of NLS0064

| | SEQ | |
|---|---|---|
| | ID | |
| Fragment | NO | Sequence |
| ref1_153668 | 17 | TAGACATTCCAACAAACCGGCAAGAGGCTCGTCCTCACTC |
| | | GAGGATTTGTTGGGACTTGCATGATGTCGAAGCGGAGCCG |
| | | TTATGACCTGGGTGCGATCATGCGCCGAGCATGGGAGATG |
| | | GCTCGGGAGGCGGCATTCGCGGTTGGCGAGCGGGCACGGA |
| | | CTCACCTTGCTGCCGCGATGCGCAGCGCGTGGGCCGAAGC |
| | | CAAGTTGGCACTCGCGCCCACGAAGACGGAGCAGGATCGT |
| | | CTCTCTCCGAGCGACATGATCGGACATGAGGACGCCTACC |
| | | AAGGCCGGGTTCTAAAATAT |
| ref1_3842117 | 18 | AAGATGGATACGACAAGCGCGATTACATTATTTGCGAAAT |
| | | AGATGGACAAATAAAAGACAAAGGACTGATGTATTTCCTT |
| | | AAATCTGGACAAGTTGACCTCTTTCACATAGAAGTCACCAC |
| | | TCCCTTTGGGACAATTTGGTGTCACGAAAACATAGAGGCCG |
| | | AACTTCTTAGCTGAATTATCGCGCTCCGGGTTCTTATGCGG |
| | | CTGAGTGAAGCGCGGGACAGCTTGCGAGCAGGGCCGCCAA |
| | | TGGCAGCCGGGATGACACAATGCTCGGTCTCCCGACGCTTC |
| | | TTCAATCGGGAGCGCT |
| ref1_3842278 | 19 | AGCTGAATTATCGCGCTCCGGGTTCTTATGCGGCTGAGTGA |
| | | AGCGCGGGACAGCTTGCGAGCAGGGCCGCCAATGGCAGCC |
| | | GGGATGACACAATGCTCGGTCTCCCGACGCTTCTTCAATCG |
| | | GGAGCGCTTCGCAGCCCGGGGCGGCGCGCTCATGCGTCAC |
| | | GACCTGGGCCCTGCGCACCTTCGCGGCCCCGCCGTCCCGGC |
| | | AGATCCCTGATGCCCCAAGTGGGCGGCCACTCCATCAAAG |
| | | AACCCCGGCCTGTGGCAGATCTCGTAGGCATACCGAGGTTC |
| | | CGCAGTGCCCCCACC |

TABLE 16
Primer and Probe Sequences for Specific Detection of NLS0064

| | SEQ | |
|---|---|---|
| | ID | |
| Primer/Probe | NO | Sequence* |
| NLS0064_ref1_153668_forward | 65 | CATGATCGCACCCAGGTCATAA |
| NLS0064_ref1_153668_reverse | 66 | CTCGTCCTCACTCGAGGATTTG |
| NLS0064_ref1_153668_probe | 67 | CGCTTCGACATCATGCAAGTCCC |
| NLS0064_ref1_3842117_forward | 68 | ACCACTCCCTTTGGGACAAT |
| NLS0064_ref1_3842117_reverse | 69 | GCTTCACTCAGCCGCATAAG |
| NLS0064_ref1_3842117_probe | 70 | AGCTGAATTATCGCG CTCC |
| NLS0064_ref1_3842278_forward | 71 | TCGGGAGACCGAGCATTGT |
| NLS0064_ref1_3842278_reverse | 72 | TATCGCGCTCCGGGTTCTTAT |
| NLS0064_ref1_3842278_probe | 73 | AAGCTGTCCCGCGCTTCAC |

*Bold and underlined letters represent the position of an LNA base

### REFERENCES

Ali et al. (2017) Front Plant Sci. 8: 750.
Baum J et al. (2007) Nature Biotech 25, 1322-1326.
Beveridge, T. (1999) J. Bacteriol. 181:4725-4733.
Bourque D, Pomerleau Y & Groleau D. (1995) Appl Microbiol Biotechnol 44: 367-376.
Cui L, Zhang C, & Xing X H. (2018) In: Kalyuzhnaya M., Xing X H. (eds) Methane Biocatalysis: Paving the Way to Sustainability. Springer, Cham., pp 199-211.
Chubiz et al. (2013) BMC Research Notes 6:183.
Gasser et al. (2009) FEMS Microbiol Ecol 70:142-150
Godge M R et al. (2008) Plant Cell Reporter 27, 209-219
Green and Ardley (2018) International Journal of Systematic and Evolutionary Microbiology 68:2727-2748.
Hane et al. (2014) PLOS Genetics 10(5): e1004281.
Hardy R W, Patro B, Pujol-Baxley C, Marx C J & Feinberg L (2018) Aquaculture Research 49 (6):2218-2224.
Huang et al. (2006) PNAS 103:14302-14306.
Karakas M (2008) African J Biotech 7, 2530-2534.
Koressaar et al. (2007) Bioinformatics Volume 23, Pages 1289-1291 at https://doi.org/10.1093/bioinformatics/btm091.
Lu, T. et al. (2012). DFVF: database of fungal virulence factors. Database: The Journal of Biological Databases and Curation, bas032).
Majumdar et al. (2017) Front. Plant Sci.: https site doi.org/10.3389/fpls.2017.00200.
Marx and Lidstrom (2001) Microbiology 147:2065-2075.
Marx and Lidstrom (2004) Microbiology 150: 9-19.
Marx, C J (2008) BMC Research Notes 1:1.
McDonald I R and Murrell J C (1997) Appl and Environ Microbiol 63, 3218-3224.
Rovere C V et al. (2001)Arch Virol 146, 1337-1353.
Salam L B, Obayori O S & Raji A S (2015) Petroleum Science and Technology 33 (2):186-195.
Schepf et al. (1998)Microbiol. Mol. Biol. Rev. 62(3):775-806.
Swain et al. (2011) Mol Plant Pathol., 12:855-65.
Sy A, Giraud E, Jourand P, Garcia N, Willems A, De Lajudie P, Prin Y, Neyra M, Gillis M, Boivin-Masson C, & Dreyfus B (2001) Jour. Bacterial. 183(1):214-220.
Toyama H, Anthony C, & Lidstrom, M (1998) FEMS Microbiol. Lett. 166:1-7.
Ueda S et al. (Applied And Environmental Microbiology 57(4): 924-926.
Untergasser et al. (2012) Nucleic Acids Research, Volume 40, Issue 15, Page e115, at https://doi.org/10.1093/nar/gks596 van Schie and Takken (2014) Annual Review of Phytopathology 52:551-581.
Viterbo et al. (2018) Biology Methods and Protocols, pp 1-6.
Yonemitsu H, Shiozaki E, Hitotsuda F (2016) Biosci Biotechnol Biochem 80:2264-2270.
Zhang, M and Lidstrom, M (2003) Microbiology 149:1033-40.
Zhang H et al. (2012) PLoS ONE 7, e49262. doi:10.1371/joumal.pone.0049262.

Having illustrated and described the principles of the present invention, it should be apparent to persons skilled in the art that the invention can be modified in arrangement and detail without departing from such principles.

The inclusion of various references herein is not to be construed as any admission by the Applicants that the references constitute prior art. Applicants expressly reserve their right to challenge any allegations of unpatentability of inventions disclosed herein over the references included herein

Although the materials and methods of this invention have been described in terms of various embodiments and illustrative examples, it will be apparent to those of skill in the art that variations can be applied to the materials and methods described herein without departing from the concept, spirit and scope of the invention. All such similar substitutes and modifications apparent to those skilled in the art are deemed to be within the spirit, scope and concept of the invention as defined by the appended claims

What is claimed is:

1. A method of producing a transconjugant Methylobacterium isolate, comprising:
incubating (i) a donor Methlyobacterium isolate comprising a mobilizable plasmid containing a marker, and (ii) a recipient Methlyobacterium isolate; wherein the mobilizable plasmid has an origin of replication functional in the recipient Methlyobacterium isolate; wherein said mobilizable plasmid is transferred from said donor Methylobacterium isolate to said recipient Methlyobacterium isolate; and
screening cells of said recipient Methlyobacterium isolate for the presence of the mobilizable plasmid marker to identify a transconjugant Methylobacterium isolate.

2. The method of claim 1, wherein said marker is a selectable marker.

3. The method of claim 2, wherein said selectable marker is a gene encoding resistance to an antibiotic.

4. The method of claim 1, wherein said marker is a genetic sequence marker.

5. The method of claim 1, wherein said marker is a screenable marker.

6. The method of claim 5, wherein said screenable marker encodes a fluorescent protein.

7. The method of claim 1, wherein said mobilizable plasmid is a native Methlyobacterium plasmid.

8. The method of claim 1, wherein said method further comprises the use of a helper strain, wherein said helper strain encodes conjugation transfer functions.

9. The method of claim 1, wherein said recipient Methylobacterium isolate contains a mutation in the carotenoid biosynthesis pathway.

10. The method of claim 9, wherein said mutation results in loss of function of crtI.

11. The method of claim 1, wherein said origin of replication is an RK2 origin of replication.

12. A method of producing a population of transconjugant Methlyobacterium isolates, comprising the steps of
(i) incubating a composition comprising a first donor Methylobacterium isolate comprising a mobilizable plasmid containing an origin of replication functional in Methlyobacterium and a marker, and one or more recipient Methlyobacterium isolates under conditions wherein said mobilizable plasmid is transferred from said donor Methylobacterium isolate to said recipient Methlyobacterium isolate or isolates; and
(ii) screening cells of said recipient Methlyobacterium isolate or isolates for the presence of the mobilizable plasmid marker to identify one or more transconjugant Methlyobacterium isolates.

13. The method of claim 12, wherein said marker is a selectable marker or screenable marker.

14. The method of claim 12, wherein said composition comprises a one or more additional donor Methlyobacterium isolates comprising a mobilizable plasmid containing an origin of replication functional in Methylobacterium and a marker.

15. The method of claim 14, wherein the marker on the mobilizable plasmid in said first donor Methlyobacterium isolate is the same marker as on the mobilizable plasmid in said one or more additional Methlyobacterium isolates.

16. The method of claim 15, wherein the marker on the mobilizable plasmid in said first donor Methlyobacterium isolate is a different marker than the marker on the mobilizable plasmid in said one or more additional donor Methlyobacterium isolates.

17. The method of claim 14, wherein the mobilizable plasmids of said first and additional donor Methlyobacterium isolates each comprise a different marker.

18. A method of producing a transformed Methlyobacterium isolate, comprising:
transforming a recipient Methlyobacterium isolate with a plasmid having an origin of replication functional in the recipient Methylobacterium isolate and a marker; wherein said plasmid is transferred to said recipient Methlyobacterium isolate; and screening cells of said recipient Methlyobacterium isolate for the presence of the marker to identify a transformed Methlyobacterium isolate.

19. The method of claim 18, wherein said marker is a genetic sequence marker.

20. The method of claim 18, wherein said plasmid is a native Methylobacterium plasmid.

21. The method of claim 18, wherein transforming is selected from the group consisting of electroporation, heat shock, ultra-sound, and transduction.

22. A Methylobacterium comprising a recombinant DNA construct wherein a promoter is operably linked to a heterologous sequence encoding a nucleic acid that can trigger an RNAi response.

23. The Methylobacterium of claim 22, wherein said RNAi response inhibits expression of a target plant pest or plant pathogen gene.

24. The Methylobacterium of claim 22, wherein said Methylobacterium further comprises a recombinant DNA construct wherein a promoter is operably linked to a heterologous sequence comprising a nucleic acid that encodes a pesticidal or herbicide tolerance protein.

25. The Methylobacterium of claim 22, wherein said RNAi response inhibits expression of a target plant gene.

26. The Methylobacterium of claim 22, wherein said promoter is an inducible promoter.

27. The Methylobacterium of claim 26, wherein said inducible promoter is a glyphosate inducible promoter.

28. The Methylobacterium of claim 27, wherein said glyphosate inducible promoter is selected from the group consisting of a trp, pheA, tyrA, tyrB, aroA, aroB, aroC, aroD, aroE, aroF, aroG, aroH, aroK, and an aroL promoter.

29. The Methylobacterium of any one of claims 22-28, wherein said Methylobacterium further comprises a recombinant DNA construct wherein an inducible promoter is operably linked to a heterologous sequence that provides for partial or complete lysis of said Methylobacterium upon exposure to an agent that induces the promoter.

30. The Methylobacterium of claim 29, wherein said inducible promoter that is operably linked to a heterologous sequence that provides for partial or complete lysis of said Methylobacterium is a glyphosate inducible promoter.

31. The Methylobacterium of claim 30, wherein said glyphosate inducible promoter that is operably linked to a heterologous sequence that provides for partial or complete lysis of said Methylobacterium is selected from the group consisting of an trp, pheA, tyrA, tyrB, aroA, aroB, aroC, aroD, aroE, aroF, aroG, aroH, aroK, and an aroL promoter.

32. The Methylobacterium of claim 29, wherein said heterologous sequence that provides for partial or complete lysis of said Methylobacterium encodes an enzyme selected from the group consisting of lysozyme, a 26 kD peptidoglycan hydrolase, an N-acetylmuramidase, an N-acetylglucosaminidase, an N-acetylmuramyl-1-alanine amidases, and an endotransglycosidase.

33. A composition comprising the Methylobacterium of any one of claims 22-32 and at least one agriculturally acceptable excipient or adjuvant.

34. A transformed Methylobacterium strain that comprises a selected host Methylobacterium strain or variant thereof comprising:
i) a first recombinant DNA construct wherein a promoter is operably linked to at least one heterologous sequence encoding a nucleic acid that can trigger an RNAi response, and
ii) a second recombinant DNA construct wherein a promoter is operably linked to a heterologous sequence comprising a nucleic acid that encodes a pesticidal or herbicide tolerance protein.

35. The transformed Methylobacterium of claim 34, wherein said RNAi response inhibits expression of a target plant pest gene and wherein said pesticidal protein is active against a target plant pest comprising the target plant pest gene.

36. The transformed Methylobacterium of claim 35, wherein said target plant pest is an insect pest or a pest that causes a plant disease.

37. The transformed Methylobacterium of claim 36, wherein said insect pest is a Coleopteran, Lepidopteran, and/or Hemipteran species pest.

38. The transformed Methylobacterium of claim 36, wherein said pest that causes a plant disease is a fungus, bacteria, virus and/or nematode pest.

39. The transformed Methylobacterium of claim 34, wherein said RNAi response inhibits expression of a gene in a first target plant pest and wherein said pesticidal protein is active against a second target plant pest.

40. The transformed Methylobacterium of claim 39, wherein said first and second target plant pests are insect pests.

41. The transformed Methylobacterium of claim 40, wherein said insect pests are Coleopteran, Lepidopteran, and/or Hemipteran species pests.

42. The transformed Methylobacterium of claim 39, wherein said first and second target plant pests are pests that cause a plant disease.

43. The transformed Methylobacterium of claim 39, wherein said pests that cause a plant disease are fungi, bacteria, virus and/or nematode pests.

44. The transformed Methylobacterium strain of any one of claims 34-43, wherein said selected host Methylobacterium strain or variant thereof exhibits or is selected for improved desiccation tolerance, improved agricultural chemistry tolerance, and/or improved colonization efficiency in comparison to a control Methylobacterium strain.

45. The transformed Methylobacterium strain of claim 44, wherein said selected host Methylobacterium strain or variant thereof is an effective colonizer of a plant shoot.

46. The transformed Methylobacterium strain of claim 45, wherein said plant is soy and said selected host Methylobacterium strain or variant thereof is NLS0064 or a variant thereof.

47. The transformed Methylobacterium strain of claim 44, wherein said selected host Methylobacterium strain or variant thereof is an effective colonizer of plant roots.

48. The transformed Methylobacterium strain of claim 47, wherein said plant is corn and said selected host Methylobacterium strain or variant thereof is NLS0042 or a variant thereof.

49. The transformed Methylobacterium strain of any one of claims 34-43, wherein said selected host Methylobacterium strain or variant thereof is a mutant strain lacking RNAse III activity.

50. The transformed Methylobacterium strain of claim 49, wherein said selected host Methylobacterium strain or variant thereof is NLS0476 or a variant thereof.

51. A composition comprising the transformed Methylobacterium of any one of claims 34-43, and at least one agriculturally acceptable excipient or adjuvant.

52. A method of altering a phenotypic trait in a host plant comprising the step of applying the Methylobacterium of any one of claims 22-32, the composition of claim 33, or the transformed Methylobacterium of any one of claims 34-43 to a plant or a plant part.

53. The method of claim 52, wherein said plant part is a seed.

54. The method of claim 52, wherein the alteration in the phenotypic trait is increased in comparison to a control plant to which a Methylobacterium lacking a recombinant DNA construct had been applied.

55. A method of altering a phenotypic trait in a host plant comprising the step of applying the composition of claim 33, to a plant or a plant part.

56. The method of claim 55, wherein said plant part is a seed.

57. A method of altering a phenotypic trait in a host plant comprising the step of applying the composition of claim 51, to a plant or a plant part.

58. The method of claim 57, wherein said plant part is a seed.

59. A method for inhibiting a plant pest in a host plant comprising the step of applying the Methylobacterium of any one of claims 22-32 or the transformed Methylobacterium of any one of claims 34-43 to a plant, a plant part, and/or to soil in which the plant will be grown or plant part deposited.

60. The method of claim 59, wherein said plant part is a seed.

61. The method of claim 59, wherein the inhibition of the plant pest is increased in comparison to a control plant to which a Methylobacterium lacking a recombinant DNA construct had been applied.

62. A method for inhibiting a plant pest in a host plant comprising the step of applying the composition of claim 33 to a plant, a plant part, and/or to soil in which the plant will be grown or plant part deposited.

63. The method of claim 62, wherein said plant part is a seed.

64. The method of claim 62, wherein the inhibition of the plant pest is increased in comparison to a control plant to which a composition containing Methylobacterium lacking a recombinant DNA construct had been applied.

65. A method for inhibiting a plant pest in a host plant comprising the step of applying the composition of claim 51 to a plant, a plant part, and/or to soil in which the plant will be grown or plant part deposited.

66. The method of claim 65, wherein said plant part is a seed.

67. The method of claim 65, wherein the inhibition of the plant pest is increased in comparison to a control plant to which a composition containing Methylobacterium lacking a recombinant DNA construct had been applied.

68. A method of detecting the presence of (a)Methylobacterium strain NLS0042 or a variant thereof; or (b) NLS0064 a variant thereof in a sample comprising detecting the presence in the sample of a nucleic acid comprising or located within: (i) SEQ ID NO:14, 15, and/or 16; or (ii) SEQ ID NO: 17, 18, or 19, respectively.

69. The method of claim 68, wherein the detecting of the nucleic acid comprises a polymerase chain reaction, branched DNA, ligase chain reaction, transcription mediated amplification (TMA), nucleic acid sequence-based amplification (NASBA), nanopore-, mass spectroscopy, hybridization, or direct sequencing based method, or any combination thereof.

70. The method of claim 68, said detection comprises the steps of:
(i) contacting the sample or DNA obtained therefrom with a DNA primer pair, wherein said primer pair comprises forward and reverse primers for amplification of a DNA fragment comprising or located within SEQ ID NO:14, 15, 16, 17, 18, or 19, thereby generating a DNA fragment,
(ii) contacting said DNA fragment with a probe specific for the presence of said DNA fragment, and
(iii) comparing the results of said contacting with positive and negative controls to determine the presence of in said sample.

71. The method of claim 68, wherein said sample is a plant material that was treated with one or more of Methylobacterium strains selected from NLS0042 or NLS0064.

72. The method of claim 68, wherein said plant material is leaves, roots or seeds.

73. The method of claim 68, wherein the plant material is a processed plant product from a plant treated with one or more Methylobacterium strains selected from NLS0042 or NLS0064.

74. The method of claim 68, wherein said sample is a soil sample.

75. A plant part which is coated or at least partially coated with a composition comprising the Methylobacterium of any one of claims 22-32, the composition of claim 33, or the transformed Methylobacterium of any one of claims 34-43.

76. The plant part of claim 75, wherein the plant part is a seed, leaf, root, stem, tuber, flower, or fruit.

77. The plant part of claim 75, wherein the plant part is a corn, soybean, Brassica sp., alfalfa, rice, rye, wheat, barley, oats, sorghum, millet, sunflower, safflower, tobacco, potato, peanut, or cotton plant part


SEQUENCE LISTING (73 sequences)

SEQ ID NO: 1 - DNA, 300 bp, Methylobacterium
gcaaaacgacctaatagttctacagcggcatgcgccaagtcagcgcggtgaacagtatacctgggagcaacttgtcctccgaaacccacataaaacaaattactcctggcagtgcccagtccatcaaaatcgaatacaatatttctcgaggaggcatctgtaatagcctgccaaagcaacaaagctatggcgccgttatgactttcattgcttctggtagacataaaataatatgccgatttgtgatcccaaatgtagaatattgccgcatcaattgcgccaagtttatttcggatcgat

SEQ ID NO: 2 - DNA, 300 bp, Methylobacterium
ggcgccaacggtatgatcgcatgattttcctgcggcatagcttgcgggaatggcgtatttggcgctctcctcaggaatttctaagggcatacgcaggaactctacagcacttttactggtattttgtagtgacagcggaggaggctggtgctcaaggtaatcgtgatgaagtgatccgggccattcggggcgcgtttctagtctttccaatccgcgccctgtaccacgtattacgccggaccggtctgcgccgcgccgccctcttgaccgccctaaatgtctaagagcgtctaacaaagc

SEQ ID NO: 3 - DNA, 300 bp, Methylobacterium
gacgatatcgctcatcttcactgcattgaagctggtgccgtactgcatagggatgaaaaagtgatgcggatagacggctgacgggaaagcgcctggtcgatcgaagactttgctgacgaggttgtggtagccccggatataggcatcgaaggccgggacgttgatcccatcctttgccttatcttgactggcgtcgtcgcgtgccgtcagaacgggcacgtcgcaggtcatcgaggccagcaccttgcggaacacctgcgttccgccgttgggattatcgacggcgaacgcggtggccgc

SEQ ID NO: 4 - DNA, 300 bp, Methylobacterium
gcccttctgtcaggcgatattgtataatggcgttgccccaatagaagcagccattcgtgcgagggcagcagcgacgctaggtcgaaagagcatcctaatctcgatcaagatgcgactgagatttctgatgaaaatatctagacacaagcaaagctggtgaaattacaacgatcatggcgacaattgcggccaattcggccggaacttgaaggaacataaaaatgaatattacaaatataccgcaaagcatgtagagttgctacaccaagggtcgggacgtccaaaaaaactcactgagga

SEQ ID NO: 5 - DNA, 300 bp, Methylobacterium
ggaacataaaaatgaatattacaaatataccgcaaagcatgtagagttgctacaccaagggtcgggacgtccaaaaaaactcactgaggaagtcgactggaagcacgaggcgccccccccaggagcggggcgaccggcaagggggcccgcaattgtcgccatgatcgaccagcttaggtaggatcctctttcgacctaacgaatggctgcttctattggggcaacgccattatacaatatcgcctgaccatctggaacgcggcccggtccaccggcaggttggcgacgacagcgtcggag

SEQ ID NO: 6 - DNA, 300 bp, Methylobacterium
cggcgtcgaccagccgggcgaactgcttgggcatgctctcccgcgacgccggccacagccgcgtccccgtccctccgcacaggatcatcgggtggatttgaaaggcaaaacgggacatcaggataggccgctcaggcgttggcgctgaggcgcttgatgtcggcgtcgaccatctcggtgatcagcgcctcgaggctggtctcggcctcccagccgaaggtcgccttggccttggcggggttgcccagcagcacctcgacctctgccggccggaacagcgccgggtcgacgatcaggtgg

SEQ ID NO: 7 - DNA, 300 bp, Methylobacterium
ctggacatgcgcccaccccggccaagtccgaccgcaccggcaaccgctcctgtagtcgtcgtcatcgttctcacccctgaggcggagaccgtccgctaacggggtgtctcaagcaaccgtggggcggaggaacacgcacgtagtcgcgtttcaaggttcgcacgaacgcctcggccatgccgttgctctgcgggctctccagcggcgtcgtttttggcaccaaaccaaggtcgcgggcgaagcggcgcgtgtcgcggggactgtcaggaatttcgtgtgggggcggccatagtggatccg

SEQ ID NO: 8 - DNA, 300 bp, Methylobacterium
ggaaatcggcttcaagtacgacgtcacgccggccatgcaggtcacgggtgcactgttcaatctcgagcgcgacaaccagccgttcccctcgaacgtggagtccggcctcgtccttggcgcaggtcagacacgcacccagggcgcggaaatcggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtct

SEQ ID NO: 9 - DNA, 300 bp, Methylobacterium
cggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgac

SEQ ID NO: 10 - DNA, 300 bp, Methylobacterium
acgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgacacgcgttcagcttagcgtcgagaacattttcgacaggcgttacatcatcaactccaacaacaacaacaacctcacgcctggcgcgccgagaacagtccgcgtgcaattgatcgctcggttctaaa

SEQ ID NO: 11 - DNA, 300 bp, Methylobacterium
acggtcaccccacggactgggcgagtacctcaccggtgttctatcataacgccgagttagttttcgaccgtcccttatgcgatgtaccaccggtgtcggcagccgatttcgtcccaccgggagctggcgttccggttcagaccaccatcatcggtcacgatgtctggattggacacggggccttcatctcccccggcgtgactataggaaacggcgcgatcgtcggggcccaggcggtcgtcacaagagatgtcccaccctatgcggtagttgctggcgtccccgcgaccgtacgacgat

SEQ ID NO: 12 - DNA, 300 bp, Methylobacterium
ccaataaaagcgttggccgcctgggcaacccgatccgagcctaagactcaaagcgcaagcgaacacttggtagagacagcccgccgactacggcgttccagcactctccggctttgatcggataggcattggtcaaggtgccggtggtgatgacctcgcccgccgcaagcggcgaattactcggatcagcggccagcacctcgaccaagtgtcggagcgcgaccaaagggccacgttcgaggacgtttgaggcgcgaccagtctcgatagtctcatcgtcgcggcgaagctgcacctcga

SEQ ID NO: 13 - DNA, 300 bp, Methylobacterium
cgatggcaccgacctgccatgcctctgccgtccgcgccagaatggtaaagaggacgaagggggtaaggatcgtcgctgcagtgttgagcagcgaccagagaagggggccgaacatcggcatcaaacctcgattgccactcggacgcgaagcgcgtcttgaaggagggatggaagcgaaacggccgcagagtaaccgccgacgaaagattgcacccctcatcgagcaggatcggaggtgaaggcaagcgtgggttattggtaagtgcaaaaaatataatggtagcgtcagatctagcgttc

SEQ ID NO: 14 - DNA, 300 bp, Methylobacterium
agcccacaagcctgatgcacttaactacatcctctaatgtcgcgccaatttgcttggcggcaggggatgttgtatcgtcataggcttgtctaaccggaacttgtttgccaatctctttggcgatcgcaaccgccatctcgtgttcgtcaaccatgtgcgcgttcctctaattgcactcatggtgccacgtgcacctccgatcgtctcgtgtctagaatgaaggtgggaacaaccttacacaggctttcgcgacgcgcgaatttctggtttctccgcctcggatgtgggtttgagcgcttc

SEQ ID NO: 15 - DNA, 300 bp, Methylobacterium
cttttcatttgtcatgatctcgaccaaggtattcacggcaagctcggtctgttgcttagcaagtgcctgaacttcgcgaacgatcggctctcgacccttcgggttcgagacctgtcccttttgaaaaccacgtgccctacacttttcgggatcaaggtgcgggttggctttggtcaaaattctctggcgtcccattacacgccctccgcatcatcgttcccgcgaacgatctgacccccgacttccgcgaggaagcgtgtggcgtgatcctcgaagcggaatgccacctcgaactgttcc

SEQ ID NO: 16 - DNA, 300 bp, Methylobacterium
cagcagcaagcagatcgttgaaaaccgcttgaaccgcatcttgatcgggaccggaaccaatcaggtcatctaggtaaaccgagacgtaaactcgtttgcgctcggcatctttcagaacgtccgtgatgccagaccgcattagtaccatcgtcgccaaggcgggcgactgaacgaagccgatcggcagagagtaacggggaccgcccctaatcgggttgcgaacgcaagaccacttagcaaaggttcgagcacggccgaacttcgcatggtggagagccgcggcaacacggttccgtgata

SEQ ID NO: 17 - DNA, 300 bp, Methylobacterium
tagacattccaacaaaccggcaagaggctcgtcctcactcgaggatttgttgggacttgcatgatgtcgaagcggagccgttatgacctgggtgcgatcatgcgccgagcatgggagatggctcgggaggcggcattcgcggttggcgagcgggcacggactcaccttgctgccgcgatgcgcagcgcgtgggccgaagccaagttggcactcgcgcccacgaagacggagcaggatcgtctctctccgagcgacatgatcggacatgaggacgcctaccaaggccgggttctaaaatat

SEQ ID NO: 18 - DNA, 300 bp, Methylobacterium
aagatggatacgacaagcgcgattacattatttgcgaaatagatggacaaataaaagacaaaggactgatgtatttccttaaatctggacaagttgacctctttcacatagaagtcaccactccctttgggacaatttggtgtcacgaaaacatagaggccgaacttcttagctgaattatcgcgctccgggttcttatgcggctgagtgaagcgcgggacagcttgcgagcagggccgccaatggcagccgggatgacacaatgctcggtctcccgacgcttcttcaatcgggagcgct

SEQ ID NO: 19 - DNA, 299 bp, Methylobacterium
agctgaattatcgcgctccgggttcttatgcggctgagtgaagcgcgggacagcttgcgagcagggccgccaatggcagccgggatgacacaatgctcggtctcccgacgcttcttcaatcgggagcgcttcgcagcccggggcggcgcgctcatgcgtcacgacctgggccctgcgcaccttcgcggccccgccgtcccggcagatccctgatgccccaagtgggcggccactccatcaaagaaccccggcctgtggcagatctcgtaggcataccgaggttccgcagtgcccccacc

SEQ ID NO: 20 - DNA, 779 bp, Methylobacterium
ttgaccggcccggacttggacgacgccgcgcagccccgggccaacgcccgatcccggcgggcccacaagccgcgcccgcccctgaccgcgctcgaggagcggatcggtcacagcttcggcgatcaggacctgctcacccgcgcgctgacccacaccagcaaggcgagcggccggggcggcagctaccagcgcctggaattcctcggcgaccgcgtgctcgggctcgcggtcgccgatcgcctctacggcgccttcccggaggccgacgagggcgacctctcccggcgcctggccgccctcgtccggcgcgagacctgcgcggccgtggccgcctcctgggaggtcggcccgcacctgatcctcggccagggcgaggtcatgggcggcggccggcgcaaccagacgatcctcgccgacgtctgcgaggcgatcctgggtgcgatctacctcgatgccgggttcgacgccgcccgggcggtggtcgaggcgcatttccgtcccggcgaaccgaccgcggcggcgcggggccgcgacgccaaatccgccctgcaggaatgggccatgggccgcagcctgccgatccccacctatgaggtggtggagcgcaccggtcccgaccacgcgccccggttccgcatcgccgtgcaggtcgagggcctggagcccggtctgggcgacggcacgtcgaagcggatcgccgagcaggccgccgcgcgggcgctgatggagcgggagggaatcggggcctccgcggacgcggaaccgacggagtcagcatg

SEQ ID NO: 21 - protein, 259 aa, Methylobacterium
MTGPDLDDAAQPRANARSRRAHKPRPPLTALEERIGHSFGDQDLLTRALTHTSKASGRGGSYQRLEFLGDRVLGLAVADRLYGAFPEADEGDLSRRLAALVRRETCAAVAASWEVGPHLILGQGEVMGGGRRNQTILADVCEAILGAIYLDAGFDAARAVVEAHFRPGEPTAAARGRDAKSALQEWAMGRSLPIPTYEVVERTGPDHAPRFRIAVQVEGLEPGLGDGTSKRIAEQAAARALMEREGIGASADAEPTESA

SEQ ID NO: 22 - DNA, 781 bp, Methylobacterium
atgaccggcccggacttggacgacgccgcgcagccccgggccaacgcccgatcccggcgggcccacaagccgcgcccgccgctgaccgcgctcgaagagcggatcggccacagcttcggcgatcaggacctgctcacccgcgcgctgacccacaccagcaaggcgagcggccggggcggcagctaccagcgcctggaattcctcggcgaccgcgtgctcgggctcgcggtcgccgaccgcctctacggcgccttcccggaggctgacgagggcgacctctcccggcgccttgccgccctggtccggcgcgagacctgcgcggccgtggccgcctcctgggaggtcggcccgcacctgatcctcggccagggcgaggtcatgggcggcggccggcgcaaccagacgatcctggccgacgtctgcgaggcgatcctgggtgcgatctacctcgatgccgggttcgacgccgcccgggcggtggtcgaggcgcatttccatcccggcgagccgaccgcggcggcgcggggccgcgacgccaaatccgccctgcaggaatgggccatgggccgcagcctgccgatccccacctatgaggtggtggagcgcaccggtcccgaccacgcgccccggttccgcatcgccgtgcaggtcgagggcctggagcccggtctcggcgacggcacgtcgaagcggatcgccgagcaggccgccgcgcgggcgctgatggagcgggagggaatcggggcctccgcggacgcggaaccgacggagacagcatgag

SEQ ID NO: 23 - protein, 259 aa, Methylobacterium
MTGPDLDDAAQPRANARSRRAHKPRPPLTALEERIGHSFGDQDLLTRALTHTSKASGRGGSYQRLEFLGDRVLGLAVADRLYGAFPEADEGDLSRRLAALVRRETCAAVAASWEVGPHLILGQGEVMGGGRRNQTILADVCEAILGAIYLDAGFDAARAVVEAHFHPGEPTAAARGRDAKSALQEWAMGRSLPIPTYEVVERTGPDHAPRFRIAVQVEGLEPGLGDGTSKRIAEQAAARALMEREGIGASADAEPTETA

SEQ ID NO: 24 - DNA, 772 bp, Methylobacterium
atgagcgaggctggcgaaggcgccggacgctccagccgcgcccggcgcgcgcaccggccccggccgagcctcgccgttctggaggcccgcatcggtcacgtcttcaccgacccgacgctgcttcccctggcgctcacccatgtgagcaaggccgggccgtccggccgggtcggcagctaccagcgcctggaatttctgggcgaccgggtgctggggctggccgtcgccgagacgctgtatcgggcgctgccggacgcggaggagggcgagctgtcgcggcggctggccgggctcgtgcggcgcgagacctgtgccgcggtggccgaggcctgggaggtcgggccgcatctcaatctcgggcccggcgagatccagaccggcgggcggcgcaaccagaccatcctggccgatgtctgcgaggcgatcctcggcgccgtgttcctcgatgccggctacgaggcgacgcgggccatcgtgcagcgcagcttccgtcccggcgaggacacgggagcaccgcgcgggcgcgacgcaaaatcggccttgcaggaatgggcgatggcgcgcagcctcgccatccccgtctacgaggtggtggagcgctcgggccccgatcatgccccggtcttccggatcgcggtgcgggtcgagagcattgaaccggggtacggcgagggcgcctccaagcgcgtggcggagcaggaggcggcccgggccgtgctcgaccgcgagaatatcggcgggacgcaagggaaggatggatcaggtggttagg

SEQ ID NO: 25 - protein, 256 aa, Methylobacterium
MSEAGEGAGRSSRARRAHRPRPSLAVLEARIGHVFTDPTLLPLALTHVSKAGPSGRVGSYQRLEFLGDRVLGLAVAETLYRALPDAEEGELSRRLAGLVRRETCAAVAEAWEVGPHLNLGPGEIQTGGRRNQTILADVCEAILGAVFLDAGYEATRAIVQRSFRPGEDTGAPRGRDAKSALQEWAMARSLAIPVYEVVERSGPDHAPVFRIAVRVESIEPGYGEGASKRVAEQEAARAVLDRENIGGTQGKDGSGG

SEQ ID NO: 26 - DNA, 779 bp, Methylobacterium
ttgaacggcccgaacttggacgaggctggacaggcccgaccgtcttcccgggcacggcgttcccacaagcccaggccgccgctcggtgtcctggaggagcggatcggccatagcttcgccgatcagaacctgctgacccgggcgctcacccataccagcaaggccaccgggcgcggcggcagctaccagcgcctggagttcctgggcgaccgggtgctcgggctcgccgtggccgaccgcctctacgccgcctttccggacgcggacgaaggcgacctgtcacgccgcctggccggcctcgtgcgccgggagacctgcgcggcggtggccgcttcctgggaggtcggcccgcatctgattctcggccagggcgaggtgatgggcggcggccggcgcaaccagacgatcctggccgacgtctgcgaggcgatcctcggtgcgatctacctcgatgccggcttcgaggccgcccgggtgatcgtcgaggcgcatttccatccggccgagccgatggcggcccatcgcggccgcgacgccaaatccgccctgcaggaatgggccatgggccggagcctgccgatccccgtctacgcggtggtcgagcgcaccggcccggatcatgcgcctcggttccgcattgccgtacaggtcgaggggctcgagccgggtcacggcgagggcacctcgaagcgtgtcgccgagcaggaggccgcccgcgcgctgatggagcgcgagggaatcggcaccgtcccggagacggggccgacggagacagcatg

SEQ ID NO: 27 - protein, 259 aa, Methylobacterium
MNGPNLDEAGQARPSSRARRSHKPRPPLGVLEERIGHSFADQNLLTRALTHTSKATGRGGSYQRLEFLGDRVLGLAVADRLYAAFPDADEGDLSRRLAGLVRRETCAAVAASWEVGPHLILGQGEVMGGGRRNQTILADVCEAILGAIYLDAGFEAARVIVEAHFHPAEPMAAHRGRDAKSALQEWAMGRSLPIPVYAVVERTGPDHAPRFRIAVQVEGLEPGHGEGTSKRVAEQEAARALMEREGIGTVPETGPTETA

SEQ ID NO: 28 - DNA, 772 bp, Methylobacterium
atgagcgaggacggcgaaggcgccgctcgctccagccgcgcccggcgtgcgcatcgcccgcggccgagcctcgcggtgctggaggcgcgcatcggccatgtcttcgccgacccgacgctgctgccgctggcgctcacccatgtgagcaaggccgggcagtcgggccgggtcggcagctaccagcgcctggaattcctgggcgaccgggtgctcgggctcgcggtggccgagacgctctacaaggcgctgcccggcgcggaggagggcgagctctcgcgccgtctcgccggcctcgtgcggcgcgagacctgcgccgtggtggccgaggcctgggaggtcgggccgcatctcaatctcggccccggcgagatccagaccggcgggcggcgcaaccagaccattctggccgacgtctgcgaggcgattctcggcgcggtgttcctcgacgccggctacgaggcgacgcgggcgatcgtgcagcgcgccttccgccccggcgaggaaaccgcggccccgcgcgggcgtgacgcgaaatcggccttgcaggaatgggcaatggcgcgcagcctcgcgatcccggtctacgaggtggtggagcgctcgggacccgaccacgccccggtcttccggatcgcggcgcgggtcgaaggcatcgaacccggctacggcgagggcgcctcgaagcgcgtcgccgagcaggaggcggcccgcgccgtactcgcccgcgagggcatcggcgggacgcagggacaggatggatcgagtggttgag

SEQ ID NO: 29 - protein, 256 aa, Methylobacterium
MSEDGEGAARSSRARRAHRPRPSLAVLEARIGHVFADPTLLPLALTHVSKAGQSGRVGSYQRLEFLGDRVLGLAVAETLYKALPGAEEGELSRRLAGLVRRETCAVVAEAWEVGPHLNLGPGEIQTGGRRNQTILADVCEAILGAVFLDAGYEATRAIVQRAFRPGEETAAPRGRDAKSALQEWAMARSLAIPVYEVVERSGPDHAPVFRIAARVEGIEPGYGEGASKRVAEQEAARAVLAREGIGGTQGQDGSSG

SEQ ID NO: 30 - DNA, 772 bp, Methylobacterium
atgagcgaggacggcgaaggcgccgctcgctccagccgcgcccggcgtgcgcatcgcccgcgaccgagcctcgcggtgctggaagcgcgcatcggccatgtcttcgcggacccgacgctgctgccgctggcgctcacccatgtgagcaaggccggccagtcgggccgggtcggcagctaccagcgcctcgaattcctgggcgaccgggtgctcgggctcgcggtggccgagacgctgtacaaggcgctgcccggcgcggaggagggtgagctgtcgcgccgccttgccggcctcgtacggcgcgagacctgcgccgtggtggccgaggcctgggaagtcgggccgcatctcaatctcggccccggcgagatccagaccggcgggcggcgcaaccagaccattctggccgacgtctgcgaggcgatcctcggcgcggtgttcctcgacgccggctacgaggcgacgcgggcgatcgtgcagcgcgcgttccgccccggcgaagaaaccgcggccccgcgcgggcgtgacgcgaaatcggccttgcaggaatgggcgatggcgcgcagcctcgcgatcccggtctacgaggtggtggagcgctcgggacccgaccacgccccggtcttccggatcgcggcacgggtcgaaggcatcgaacccggctacggcgagggcgcctcgaagcgcgtcgccgagcaggaggcggcccgcgccgtactcgcccgcgagggcatcggcgggacgcagggacaggatggatcgagtggttgag

SEQ ID NO: 31 - protein, 256 aa, Methylobacterium; identical to SEQ ID NO: 29

SEQ ID NO: 32 - DNA, 758 bp, Methylobacterium
gtgaccgacgacgccgcgcgtccctccgccgcagcgcggcgggggcgccgcccgaagccgccgctgggcctcttggaggcccggatcggccacagcttcgccgaccgcgccctgctggaacgggcgctgacgcatgtcagcaaggcgggcgggcgcggcggcagctaccagcgcctcgaattcctcggcgaccgggtgctcgggctggcggtcgccgacatcctctacgccgcgctgccggacgccgacgagggcgagctgtcgcgccggctcgccggcctcgtgcgccgcgagacctgcgcggcggtctcgaacgcctgggaggtcggcccccacctgagcctcggccagggcgaggtcagcggcggcgcgcggcgcaaccagacgatcctggccgatgtctgcgaggcgatcctcggcgcggtctacctcgatgccggctatctgagggcccgcgagatcgtggaggccgccttccggccagccgcgctgaccggcgccccgcgcgggcgcgacgccaaatccgccctgcaggaatgggcgatggcgcgcagtctcccgctgcccacctacgcggtggtggagcgctcggggcccgaccacgcgccgcattaccgcatcgccgcgcaggtcgaggggctggagcccgggatcggcgagggcccttcgaagcgggtcgccgaacaggaggccgcccgcgcgttgatggaacgggagggcatcgcggacgccgctccgacggaaccgaacgatggctg

SEQ ID NO: 33 - protein, 252 aa, Methylobacterium
MTDDAARPSAAARRGRRPKPPLGLLEARIGHSFADRALLERALTHVSKAGGRGGSYQRLEFLGDRVLGLAVADILYAALPDADEGELSRRLAGLVRRETCAAVSNAWEVGPHLSLGQGEVSGGARRNQTILADVCEAILGAVYLDAGYLRAREIVEAAFRPAALTGAPRGRDAKSALQEWAMARSLPLPTYAVVERSGPDHAPHYRIAAQVEGLEPGIGEGPSKRVAEQEAARALMEREGIADAAPTEPNDG

SEQ ID NO: 34 - DNA, 758 bp, Methylobacterium
gtgaccgacgacgccgcgcgtccctccgccgcagcgcggcgggggcgccgcccgaagccgccgctgggcctcttggaggcccggatcggccacagcttcgccgaccgcgccctgctggaacgggcgctgacgcatgtcagcaaggcgggcgggcgcggcggcagctaccagcgcctcgaattcctcggcgaccgggtgctcgggctggcggtcgccgacatcctctacgccgcgctgccggacgccgacgagggcgagctgtcgcgccggctcgccggcctcgtgcgccgcgagacctgcgcggcggtctcgaacgcctgggaggtcggcccccacctgagcctcggccagggcgaggtcagcggcggcgcgcggcgcaaccagacgatcctggccgatgtctgcgaggcgatcctcggcgcggtctacctcgatgccggctatctgagggcccgcgagatcgtggaggccgccttccggccggccgcgctgaccggcgccccgcgcgggcgcgacgccaaatccgccctgcaggaatgggcgatggcgcgcagtctcccgctgcccacctacgcggtggtggagcgctcggggcccgaccacgcgccgcattaccgcatcgccgcgcaggtcgaggggctggagcccgggatcggcgagggcccttcgaagcgggtcgccgaacaggaggccgcccgcgcgttgatggaacgggagggcatcgcggacgccgctccgacggaaccgaacgatggctg

SEQ ID NO: 35 - protein, 252 aa, Methylobacterium; identical to SEQ ID NO: 33

SEQ ID NO: 36 - DNA, 770 bp, Methylobacterium
ttgagcgcgtccgacgagggcgccccgcgctccagccgtgcccggcgcgcccagcggccgcggccgggcctcgacgtgttggaggcccgcatcggccatcacttcaccgattccacgctgctgccgctggcgctgacccatgtcagcaaggccggccataccggccgggtcggcagctaccagcgcctcgagttcttgggcgaccgcgtgctggggctcgcgattgccgagagcctctacaccgccttccccgaggcggaggagggcgagctgtcgcgccgtctcgccggcctcgtgcggcgggagacctgcgccgccgtggccgaggcctgggaggtcggcccccatctcaatctcggccccggcgaggtccagaccggcgggcggcgcaaccagaccatcctggccgacgtctgcgaggcgatcctcggcgcggtcttcctcgatgccggctatccggccaccaaggccatcgtgcaggcggccttccgccccggcgaggtcaccggcgccccgcgcgggcgcgacgccaaatcgtcgttgcaggaatgggcgatggcgcgcagtctcgcgattccgctctacgaggtggtggatcgctccggccccgaccacgccccggtcttccgcatcgccgtgcgggtcgatgggatcgaaccgggctacggcgagggtaattccaagcgcgtggccgagcaggaggccgcccgcgccgtgctggcccgcgagggcatcggcggcacccaggatcaggacaacacccatggctg

SEQ ID NO: 37 - protein, 256 aa, Methylobacterium
MSASDEGAPRSSRARRAQRPRPGLDVLEARIGHHFTDSTLLPLALTHVSKAGHTGRVGSYQRLEFLGDRVLGLAIAESLYTAFPEAEEGELSRRLAGLVRRETCAAVAEAWEVGPHLNLGPGEVQTGGRRNQTILADVCEAILGAVFLDAGYPATKAIVQAAFRPGEVTGAPRGRDAKSSLQEWAMARSLAIPLYEVVDRSGPDHAPVFRIAVRVDGIEPGYGEGNSKRVAEQEAARAVLAREGIGGTQDQDNTHG

SEQ ID NO: 38 - DNA, 770 bp, Methylobacterium
atgagcgaggctggcgaaggcgccggacgctccagccgcgcccggcgcgcgcaccggccccggccgagcctcgccgttctggaggcccgcatcggtcacgtcttcaccgacccgacgctgcttcccctggcgctcacccatgtgagcaaggccgggccgtccggccgggtcggcagctaccagcgcctggaatttctgggcgaccgggtgctggggctggccgtcgccgagacgctgtatcgggcgctgccggacgcggaggagggcgagctgtcgcggcggctggccgggctcgtgcggcgcgagacctgtgccgcggtggccgaggcctgggaggtcgggccgcatctcaatctcgggcccggcgagatccagaccggcgggcggcgcaaccagaccatcctggccgatgtctgcgaggcgatcctcggcgccgtgttcctcgatgccggctacgaggcgacgcgggccatcgtgcagcgcagcttccgtcccggcgaggacacgggagcaccgcgcgggcgcgacgcaaaatcggccttgcaggaatgggcgatggcgcgcagcctcgccatccccgtctacgaggtggtggagcgctcgggccccgatcatgccccggtcttccggatcgcggtgcgggtcgagagcattgaaccggggtacggcgagggcgcctccaagcgcgtggcggagcaggaggcggcccgggccgtgctcgaccgcgagaatatcggcgggacgcaagggaaggatggatcaggtggtta

SEQ ID NO: 39 - protein, 256 aa, Methylobacterium; identical to SEQ ID NO: 25

SEQ ID NO: 40 - DNA, 767 bp, Methylobacterium
ttgagcgcggacgcgacaccccttccgcgcgtctcggcgcgggccaagcgagcgcacaagccgcgcccgccgctcgccgaactcgagcagaagatcggccatattttcgcggacagggagctcctgacccgcgcgctgacccacaccagcaaagccgccggccgcaacggcagctaccagcggctcgaattcctgggcgaccgggtgctcgggctggcggtggcggagggcctttacgccgccctgccggatgccgacgagggagacctctcgcgccagctcgccggcctcgtgcggcgggagacctgcgcggccgtggccggcacctggggcgtcggcgcgcacctgatcctcggccagggcgaggtgatggggggcgggcgccgcaaccagaccatcctggccgacgtgtgcgaggcgatcctcggcgccgtctatctcgatgccggcttcgatgcggcccgcgccatcgtggaagcgcatttccgcccggccgagcccggcaccgccccgcgcgggcgcgacgcgaaatcgaccctgcaggaatgggccatgagccgaagcctgccgattcctacctatgccgtcgtggaacgcaccggccccgaccacgccccgcatttccgcatggcggtgcaggtcgaggggctggaacccggctacggtgagggcacctcgaagcgcgtggccgagcaggccgccgccggggcgctgctcgcccgcgaaggcatccatgcgatcggtgaagccgccgcggagacgacatg

SEQ ID NO: 41 - protein, 255 aa, Methylobacterium
MSADATPLPRVSARAKRAHKPRPPLAELEQKIGHIFADRELLTRALTHTSKAAGRNGSYQRLEFLGDRVLGLAVAEGLYAALPDADEGDLSRQLAGLVRRETCAAVAGTWGVGAHLILGQGEVMGGGRRNQTILADVCEAILGAVYLDAGFDAARAIVEAHFRPAEPGTAPRGRDAKSTLQEWAMSRSLPIPTYAVVERTGPDHAPHFRMAVQVEGLEPGYGEGTSKRVAEQAAAGALLAREGIHAIGEAAAETT

SEQ ID NO: 42 - DNA, 1529 bp, Methylobacterium
tgagccaggattccacggtcgccgtcatcggcagcgggctcggcggactcgccgccgcgtgcgtcgccgccgcgcggggccaccgcgtgacggtctacgacaagaacggctggctcggcggaaaggcggcggtgctccacgagggcggcttccgcttcgacatggggccgacgatcctgaccgtgccccgggtgctggagcggatcttcgcggagtccggccgctcggtccacgactacatggacctgcgccgcctggacccgcagtggcgctgcttcttcgacgacggcacgcatatcgacctgatgcaggacgtcgagcggatggccggcgacatggagcgcttcgcccccggcaccggcgagggctacaagaagttcatcaccatctccgagcatcttcacggcgtgtccgagaaattcttcttctggaagccggtcgaggacctgttcgacacgatcaacatccgcgccaacatgaacccgggcacgctgcgcgacgtactgtcactccgcatgcacgcctcggtggcctcgacgatccgcggcaaggtcaaggacgcgcggctcgcgcagatgctcgaccacttcgtgcagtatgtcggctcctcgccctacggcgcgccggcggtgctctgcgccatcgcccacatgcagacggccgacggcgtctggtacccgatgggcggcacccgcgccgtcgccgaggggctggcgaagcttgcccaggaactcggcgccacgtgcaagaccgacaccgaggtgaccgggatcgagaccgccaacggcgcggtccggggcgtgcggacggccgcgggggtcacgccctacgacgccgtgatctccaacatggactcggtccgcacctaccgcgagctcgtgggcggcgaggccgccaaggcctacgccaagaagaaccccgaaccggcctgctcgggcgtggtgctctatctcggcctgaacaagcgctacgagcacctcaaccaccacgacttcgtcttctcccgcgacccggaggaggagttcgactacatctacaacaagggcgagcctgccccggaccccaccgcctatctggcagctccctcctccaccgatccgagcgtggcgcccgagggcggcgaggcgctctacgtcctcgtgcacacgccgtatctgcggccgcaccacgactggcgtccggacgggccgctgttccagaactaccgccgcacgatcctcgacaagctcaagcgcaccgccggcatgccggacctcgaagagcgtatcgtcgtcgagcgccacctcacgccgcaggacatccacgagcgctacaaggtcctgaacggcgcgatctacggcctagcctcccacgggcgcgtgatgggggcgttcaagcccggcaaccggtcgcggcaggtgcgcggcctctacctcgcgggcggcgccgcccatccggggcccggcatgccgatggtgatgatgtcgggctggatcgccgccgacgcgcatgaccaggacgcgcgcggcgccctgcgcgcctcggcgtga

SEQ ID NO: 43 - DNA, 90 bp, Artificial
  synthetic
tgcatcccaacaacttataccatggcctacaaaaaggcaaacaatggtacttgacgactcatcacaacaattgtagttgtagattgtaat

SEQ ID NO: 44 - DNA, 1746 bp, Artificial
  synthetic
atgatcgagaccggctacaccccgatcgacatctcactgtctctaacccagttcctcctgtcggagttcgtgccgggcgccggcttcgtgctgggcctggtggacatcatctggggcattttcggcccgtcgcagtgggacgccttcccggtgcagatcgagcagctgatcaaccagcggatcgaagagttcgcccgcaaccaggccatctcgcgcctggagggcctgtcgaacctgtaccaaatctacgccgagtcgttccgcgagtgggaggccgacccgaccaacccggccctgcgcgaggagatgcgcatccagttcaacgacatgaactcggccctgaccaccgccatcccgctgctggccgtgcagaactaccaggtgccgctgctgtcggtgtacgtgcaggccgccaacctgcacctgtcggtgctgcgcgacgtgtcggtgttcggccagcgctggggcttcgacgccgccaccatcaactcgcgctacaacgacctgacccgcctgatcggcaactacaccgactacgccgtgcgctggtacaacaccggcctggagcgcgtgtggggcccggactcgcgcgactgggtgcgctacaaccagttccgccgcgagctgaccctgaccgtgctggacatcgtggccctgttctcgaactacgactcgcgccgctacccgatccggacggtgtcgcagctgacccgcgaaatctacaccaacccggtgctggagaacttcgacggctcgttccgcggcatggcccagcgcatcgagcagaacatccgccagccgcacctgatggacatcctgaactcgatcaccatctacaccgacgtgcaccgcggcttcaactactggtcgggccaccagataaccgcctcgccggtgggcttctcgggaccggagttcgccttcccgctgttcggcaacgccggcaacgcagccccgccggtgctggtgtcgctgaccggcctggggatcttccgcaccctgtcgtcgccgctgtaccgccgcatcatcctgggatcgggcccgaacaaccaggagctgttcgtcctggacggcaccgagttctcgttcgcctcgctgaccactaacctgccgtcgaccatctaccgccagcgcggcaccgtggactcgctggacgtgatcccgccgcaggacaactcggtgccgccgcgcgccggcttctcgcaccgcctgtcgcacgtgaccatgctgtcgcaggccgccggcgccgtgtacaccctgcgggccccgaccttctcgtggcagcaccgctcggccgagttcaacaacatcatcccgtcgtcgcagatcacccagatcccactgaccaagtcgaccaacctgggctcgggcacctcggtggtgaagggcccgggcttcaccggcggcgacatcctgcgccgcacctcgccgggccaaatctcgaccctgcgcgtgaacatcaccgccccgctgtcgcagcgctaccgcgtgcgcatccgctacgcctcgaccaccaacctgcagttccacacctcgatcgacggccgcccgatcaaccagggcaacttctcggccaccatgtcgtcgggctcgaatctgcaatcgggctcattccgcaccgtgggcttcaccaccccgttcaacttctcgaacggctcgtcggttttcaccctgtcggcccacgtgttcaactcgggcaacgaggtgtacatcgaccgcatcgagttcgtgccgaatctctcgtag

SEQ ID NO: 45 - DNA, 2403 bp, Artificial
  synthetic
atgaagcagaacaacaacttctcggtgcgcgccctgccgtcgttcatcgacgtgttcaacggcatctacggcttcgccaccggcatccaggacatcttcaacatgatcttcggcaccgacaccggcgacctgaccctggaggaggtgctgaagaaccaggagctgctgtacgacatctcgggcaagctggagggcatctcgggcgacctgtcggagatcatcgcccagggcaacctgaacaccgagctggccaaggagctgctgaagatcgccaacgagcagaacaacgtgctgaccgacgtgaacaacaagctgaacgccatcaactcgatgctgcacatctacctgccgaagatcaccaacatgctgtcggacgtgatgaagcagaactacgccctgtcgctgcagatcgagtacctgtcgaagcagctgcaggaaatctcggacaagctggacgtgatcaacctgaacgtgctgatcaactcgaccctgaccgagatcaccccggcctaccagcgcatcaagtacgtgaacgagaagttcgacgagctgaccctggccaccgagaagaccctgcgcgccaagcagggctcggaggacatcatcgccaacgacaccctggagaacctgaccgagctgaccgagctggccaagtcggtgaccaagaacgacatggactcgttcgagttctacctgcacaccttccacgacgtgctgatcggcaacaacctgttcggccgctcggccctgaagaccgccgccgagctgatcaccaaggacgagatcaagacctcgggctcggagatcggcaaggtgtactcgttcctgatcgtgctgacctgcctgcaggccaaggccttcctgaccctgaccgcctgccgcaagctgctgggcctgtcggacatcgactacaccaacatcctgaaccagcacctgaacgacgagaagaacgtgttccgcgacaacatcctgccgaccctgtcgaacaagttctcgaacccgaactacgtgaagaccatcggctcggacaactacgccaaggtgatcctggaggccgagccgggctacgccctggtgggcttcgagatcatcaacgaccgcatcccggtgctgaaggcctacaaggccaagctgaagcagaactaccaggtggaccaccagtcgctgtcggagatcgtgtacctggacatcgacaagctgttctgcccgaagaactcggagcagaagtactacaccaagtcgctgaccttcccggacggctacgtgatcaccaagatcaccttcgagaagaagctgaacaacctgcgctacgaggccaccgccaacttctacgacccgtcgaccggcgacatcgacctgaacgagaagcaggtggagtcgaccttcctgcaggccgactacatctcgatcaacgtgtcggacgacgacggcgtgtacatgccgctgggcgtgatctcggagaccttcctgtcgccgatcaactcgttcgagctggaggtggacgagaagtcgaagatcctgaccctgacctgcaagtcgtacctgcgcgagtacctgctggagtcggacctgatcaacaaggagacctcgctgatcgccccgccgaacgtgttcatctcgaacatcgtggagaactggaacatcgaggccgacaacctggagccgtgggtggccaacaacaagaacgcctacgtggactcgaccggcggcatcgagggctcgaaggccctgttcacccagggcgacggcgagttctcgcagttcatcggcgacaagctgaagccgaacaccgactacatcatccagtacaccgtgaagggcaagccggccatctacctgaagaacaagaacaccggctacaccatgtacgaggacaccaacggctcgtcggaggagttccagaccatcgccgtgaactacacctcggagaccgacccgtcgcagacccacctggtgttcaagtcgcagtcgggctacgaggcctggggcgacaacttcatcatcctggagtgcaaggccttcgagaccccggagggcccggagctgatcaagttcgacgactggatctcgttcggcaccacctacatccgcgacgacgtgctgaccatcgacccgtcgcgcggcggctacttccgccagtcgctgaagctggactcgtactcgacctacaacctgtcgttctcgttctcgggcctgtgggccaaggtgatcatcaagaactcgcacggcgtggtgctgttcgagaaggtgtcgcagcagtcgtcgtacgtggacatctcggagtcgttcaccaccacctcgaacaaggagggcttcttcatcgagctgaccggcgactcgcgcggcggcttcggctcgttccgcgacttctcgatgaaggagaagttcgagaatctctcgtag

SEQ ID NO: 46 - DNA, 3546 bp, Artificial
  synthetic
atggacaacaacccgaacatcaacgagtgcatcccgtacaactggctgtcgaacccggaggtggaggtgctgggcggcgagcgcatcgagaccggctacaccccgatcgacatctcgctgtcgctgacccagttcctgctgtcggagttcgtgccgggcgccggcttcgtgctgggcctggtggacatcatctggggcatcttcggcccgtcgcagtgggacgccttcctggtgcagatcgagcagctgatcaaccagcggatcgaggagttcgcccgcaaccaggccatctcgcgcctggagggcctgtcgaacctgtaccaaatctacgccgagtcgttccgtgagtgggaggctgacccgaccaacccggccctgcgcgaggagatgcgcatccagttcaacgacatgaactcggccctgaccaccgccatcccgctgttcgccgtgcagaactaccaggtgccgctgctgtcggtgtacgtgcaggccgccaacctgcacctctcggtgctgcgcgacgtgtcggtgttcggccagcgctggggcttcgacgccgccaccatcaactcgcggtacaacgacctgacccgcctgatcggcaactacaccgactacgccgtgcgctggtacaacaccggcctggagcgcgtgtggggcccggactcgcgcgactgggtgcgctacaaccagttccgccgcgagctgaccctgaccgtgctggacatcgtggccctgttcccgaactacgactcgcgccgctacccgatccgcaccgtgtcgcagctgacccgcgaaatctacaccaacccggtgctggagaacttcgacggctcgttccgcggctcggcccagggcatcgagcgctcgatccgctcgccgcacctgatggacatcctgaactcgatcaacatctacaccgacgcccaccgcggctactactactggtcgggccaccagatcaaggcctcgccggtgggcttctcgggcccggagttcaccttcccgctgtacggcaccatgggcaacgaggccccgcagcagcggatcgtggcccacctgggccagggcgtgtaccgcatcctgtcgtcgaccctgtaccgccgcccgttcaacatcggcatcaacaaccagcagctgtccgtgctggacggcaccgagttcgcctacggcacctcgtcgaacctgccgtcggccgtgtatcgcaagtcgggcaccgtggactcgctggacgagatcccgccgcagaacaacaacgtgccgccgcgccaaggcttctcgcaccgcctgtcgcacgtgtcgatgttccgctccgggttctcgaactcctcggtgtcgatcatccgcgccccgatgttctcgtggatccaccgctcggccaagttctccaacatcatcgcctcggactcgatcacccagatcccggccgtgaagggcaacttcctgttcaacggctcggtgatctccggcccgggtttcaccggcggcgacctggtgcgcctgaactcgtccggcaacaacatccagaaccgcggctacatcgaggtgccgatccacttcccgtcgacctcgacccgctaccgcgtgcgcgtgcgctacgcctcggtgaccccgatccacctgaacgtgaactggggcaactcgtcgatcttctcgaataccgtgccggccaccgccacctcgctggacaacctgcagtcgtcggacttcggctacttcgagtcggccaacgccttcacctcgctgctgggcaacatcgtgggcgtgcgcaacttctcgggcaccgccggcgtgatcatcgaccgctttgagttcatcccggtgaccgccaccctggaggccgagtacaacctggagcgcgcccagaaggccgtgaacgccctgttcacctcgaccaaccagctgggcctgaagaccaacgtgaccgactaccacatcgaccaggtgtcgaacctggtgacctacctgtcggacgagttctgcctggacgagaagcgcgagctgtcggagaaggtgaagcacgccaagcgcctgtcggacgagcgcaacctgctgcaggactcgaacttcaaggacatcaaccgccagccggagcgcggctggggcggctcgaccggcatcaccatccagggcggcgacgacgtgttcaaggagaactacgtgaccctgtcgggcaccttcgacgagtgctacccgacctacctgtaccagaagatcgacgagtcgaagctgaaggccttcacccgctaccagctgcgcggctacatcgaggactcgcaggacctggaaatctacctgatccgctacaacgccaagcacgagaccgtgaacgtgccgggcaccggctcgctgtggccgctgtcggcccagtcgccgatcggcaagtgcggcgagccgaaccgctgcgccccgcacctggagtggaacccggacctggactgctcgtgccgcgacggcgagaagtgcgcccaccactcgcaccacttctcgctggacatcgacgtgggctgcaccgacctgaacgaggacctgggcgtgtgggtgatcttcaagatcaagacccaggacggccacgcccgcctgggcaacctggagttcctggaggagaagccgctggtgggcgaggccctggcccgcgtgaagcgcgccgagaagaagtggcgcgacaagcgcgagaagctggagtgggagaccaacatcgtgtacaaggaggccaaggagtcggtggacgccctgttcgtgaactcgcagtacgaccagctgcaggccgacaccaacatcgccatgatccacgccgccgacaagcgcgtgcactcgatccgcgaggcctacctgccggagctgtcggtgatcccgggcgtgaacgccgccatcttcgaggagctggagggccgcatcttcaccgccttctcgctgtacgacgcccgcaacgtgatcaagaacggcgacttcaacaacggcctgtcgtgctggaacgtgaagggccacgtggacgtggaggagcagaacaaccagcgctcggtgctggtggtgccggagtgggaggccgaggtgtcgcaggaggtgcgcgtgtgcccgggccgcggctacatcctgcgcgtgaccgcctacaaggagggctacggcgagggctgcgtgaccatccacgagatcgagaacaacaccgacgagctgaagttctcgaactgcgtggaggaggaaatctacccgaacaacaccgtgacctgcaacgactacaccgtgaaccaggaggagtacggcggcgcctacacctcgcgcaaccgcggctacaacgaggccccgtcggtgccggccgactacgcctcggtgtacgaggagaagtcgtacaccgacggccgccgcgagaacccgtgcgagttcaaccgcggctaccgcgactacaccccgctgccggtgggctacgtgaccaaggagctggagtacttcccggagaccgacaaggtgtggatcgagatcggcgagaccgagggcaccttcatcgtggactcggtggagctgctgctgatggaggagaatctctcgtag

SEQ ID NO: 47 - DNA, 23 bp, Artificial
  synthetic
cctcaccggtgttctatcataac

SEQ ID NO: 48 - DNA, 20 bp, Artificial
  synthetic
ccgatgatggtggtctgaac

SEQ ID NO: 49 - DNA, 21 bp, Artificial
  synthetic
cgtcccttatgcgatgtacca

SEQ ID NO: 50 - DNA, 22 bp, Artificial
  synthetic
gatccgagcctaagactcaaag

SEQ ID NO: 51 - DNA, 21 bp, Artificial
  synthetic
gaccaatgcctatccgatcaa

SEQ ID NO: 52 - DNA, 20 bp, Artificial
  synthetic
aacacttggtagagacagcc

SEQ ID NO: 53 - DNA, 21 bp, Artificial
  synthetic
aaggagggatggaagcgaaac

SEQ ID NO: 54 - DNA, 19 bp, Artificial
  synthetic
ataacccacgcttgccttc

SEQ ID NO: 55 - DNA, 21 bp, Artificial
  synthetic
cgcagagtaaccgccgacgaa

SEQ ID NO: 56 - DNA, 22 bp, Artificial
  synthetic
aagcctgtgtaaggttgttccc

SEQ ID NO: 57 - DNA, 21 bp, Artificial
  synthetic
ccatgtgcgcgttcctctaat

SEQ ID NO: 58 - DNA, 21 bp, Artificial
  synthetic
acctccgatcgtctcgtgtct

SEQ ID NO: 59 - DNA, 20 bp, Artificial
  synthetic
gtaatgggacgccagagaat

SEQ ID NO: 60 - DNA, 19 bp, Artificial
  synthetic
tgcttagcaagtgcctgaa

SEQ ID NO: 61 - DNA, 20 bp, Artificial
  synthetic
aagccaacccgcaccttgat

SEQ ID NO: 62 - DNA, 20 bp, Artificial
  synthetic
ccgtgctcgaacctttgcta

SEQ ID NO: 63 - DNA, 23 bp, Artificial
  synthetic
cagaccgcattagtaccatcgtc

SEQ ID NO: 64 - DNA, 19 bp, Artificial
  synthetic
cgggttgcgaacgcaagac

SEQ ID NO: 65 - DNA, 22 bp, Artificial
  synthetic
catgatcgcacccaggtcataa

SEQ ID NO: 66 - DNA, 22 bp, Artificial
  synthetic
ctcgtcctcactcgaggatttg

SEQ ID NO: 67 - DNA, 23 bp, Artificial
  synthetic
cgcttcgacatcatgcaagtccc

SEQ ID NO: 68 - DNA, 20 bp, Artificial
  synthetic
accactccctttgggacaat

SEQ ID NO: 69 - DNA, 20 bp, Artificial
  synthetic
gcttcactcagccgcataag

SEQ ID NO: 70 - DNA, 19 bp, Artificial
  synthetic
agctgaattatcgcgctcc

SEQ ID NO: 71 - DNA, 19 bp, Artificial
  synthetic
tcgggagaccgagcattgt

SEQ ID NO: 72 - DNA, 21 bp, Artificial
  synthetic
tatcgcgctccgggttcttat

SEQ ID NO: 73 - DNA, 19 bp, Artificial
  synthetic
aagctgtcccgcgcttcac